(19) United States
(12) Patent Application Publication
(10) Pub. No.: US 2026/0053935 A1
(43) Pub. Date: Feb. 26, 2026

(54) METHODS AND COMPOSITIONS FOR TREATING CANCER WITH CANCER-BINDING ADJUVANTS

(71) Applicant: THE UNIVERSITY OF CHICAGO, Chicago, IL (US)

(72) Inventors: Jeffrey A. HUBBELL, Chicago, IL (US); Anna J. SLEZAK, Chicago, IL (US); Levi Y. BENNISH, Chicago, IL (US); Claudia BATTISTELLA, Chicago, IL (US)

(73) Assignee: THE UNIVERSITY OF CHICAGO, Chicago, IL (US)

(21) Appl. No.: 19/103,723

(22) PCT Filed: Aug. 17, 2023
(86) PCT No.: PCT/US2023/072386

§ 371 Date: Feb. 13, 2025

Related U.S. Application Data
(60) Provisional application No. 63/399,129, filed on Aug. 18, 2022.

(51) Int. Cl. A61K 47/59; A61K 9/00; A61K 31/7068; A61P 35/02; C07K 16/28

(52) U.S. Cl. CPC A61K 47/593; A61K 9/0019; A61K 31/7068; A61P 35/02; C07K 16/2818

(57) ABSTRACT

Disclosed herein are compositions comprising immune-activating agents that bind the tumor cell surface in the absence of a tumor-targeting protein component. Described herein are methods and compositions for targeting a TLR or STING agonist to tumor cells and/or cells in the tumor microenvironment. It is hypothesized that the metabolic stress of tumor growth also produces an excess of unpaired cysteines on cell surface proteins relative to the rest of the body. Therefore, this chemistry can be used with compounds that would preferentially target unpaired cysteines on cell surface proteins.

### CROSS-REFERENCE TO RELATED APPLICATIONS

This application claims the benefit of priority to U.S. Provisional Patent Application Ser. No. 63/399,129, filed Aug. 18, 2022, hereby incorporated by reference in its entirety.

### SEQUENCE LISTING

The application contains a Sequence Listing in compliance with ST. 26 format and is hereby incorporated by reference in its entirety. Said Sequence Listing, created on Aug. 17, 2023 is named ARCDP0774WO.xml and is 8.4 kilobytes in size.

### BACKGROUND

### Field of the Invention

The invention generally relates to the field of medicine. More particularly, it concerns compositions and methods involving compounds for treating cancers.

### Background

In recent years, immunotherapies have emerged as promising anti-cancer therapeutics, led by the development of immune checkpoint inhibitors (ICIs). ICIs have shown efficacy but only provide a significant survival benefit in a small fraction of patients. Further immune activation in the form of adjuvants can provide additional efficacy in the case of immunologically “cold” tumors, which are often resistant to ICIs. Toll-like receptor (TLR) agonists can powerfully trigger the innate immune system and potentiate an antigen-specific T cell response to fight against tumors. However, their clinical translation has been limited due to a narrow therapeutic window, in which non-specific immune activation can produce excessive systemic inflammation and lead to chronic disease. To overcome these challenges, there is a need in the art for therapies that provide for cancer targeting. Previous technologies have used tumor-targeting proteins, such as a tumor-associated cell-surface marker, to target adjuvants to the tumor microenvironment. However, the immunogenicity of this platform, especially the development of antibodies against the tumor-targeting protein, limited the number of doses that could be used in preclinical models, presumably due to induction of immune reactions to the administered protein component of the conjugate therapeutic. In order to develop a fully synthetic, tumor cell-surface binding material, tumor-intrinsic cues must be considered. Therefore, there is a need in the art for targeting agents to the tumor microenvironment without the use of immunogenic polypeptides that can cause systemic inflammation and chronic disease.

### SUMMARY OF INVENTION

Here, the inventors seek to address the aforementioned issues in the art by developing immune-activating agents that bind the tumor cell surface in the absence of a tumor-targeting protein component. Described herein are methods and compositions for targeting a TLR or STING agonist to tumor cells and/or cells in the tumor microenvironment. The inventors hypothesized that the metabolic stress of tumor growth also produces an excess of unpaired cysteines on cell surface proteins relative to the rest of the body. Therefore, the inventors exploited this chemistry with compounds that would preferentially target unpaired cysteines on cell surface proteins.

Described herein is a polymer comprising the structure (I):

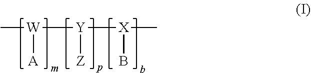

W, Y, and X are each independently a monomer unit of a polymer; A comprises a group that binds to an Antigen Presenting Cell (APC) mannose receptor; Z comprises a TLR or STING agonist; B comprises a pyridyl disulfide moiety or a maleimide moiety; m is an integer ranging from 0 to 150; p is an integer ranging from 0 to 10; and b is an integer ranging from 1 to 10.

m may include or exclude 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150, or any range derivable therein. p may include or exclude 0, 1, 2, 3, 4, 5, 6, 7, 8, 9, or 10, or any derivable range therein. b may include or exclude 1, 2, 3, 4, 5, 6, 7, 8, 9, or 10, or any derivable range therein.

Also described is a compound comprising:

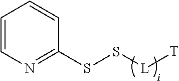

wherein L is a linker, T is a TLR or STING agonist, and i is 0 or 1.

Also described is a compound comprising:

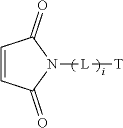

wherein L is a linker, T is a TLR or STING agonist, and i is 0 or 1. Also provided are compositions comprising the polymers and compounds of the disclosure. Methods include a method for treating cancer in a subject comprising administering a polymer, compound, or composition of the disclosure to the subject. Also described is a method for targeting a TLR or STING agonist to a tumor in a subject comprising administering a polymer, compound, or composition of the disclosure to the subject. Methods include a method for increasing the accumulation of a TLR or STING agonist in a tumor in a subject, the method comprising administering a polymer, compound, or composition of the disclosure to the subject.

W, Y, and X may be each independently polymerized monomer units that include or exclude polyacrylate, a polyacrylamide, a saturated polyolefin, a polyamide, a peptide, a polypeptide, an unsaturated olefin formed by ring opening metathesis polymerization, a siloxane, a polysiloxane, a polyether, a polysaccharide, a polyoxazoline, a polyimine, a polyvinyl derivative or any combination thereof. W, Y, and X may be independently selected from or may exclude acrylic acid, methacrylic acid, acrylamide, methacrylamide, N-(2-hydroxypropyl) methacrylamide, N-(2-hydroxyethyl) methacrylamide, natural and un-natural amino acids, silanols, monosaccharides, glycols, substituted and unsubstituted 2-oxazolines N-substituted and unsubstituted aziridine, substituted and unsubstituted 2-ethyl-2-oxazoline.

The TLR agonist may have the general structure (II),

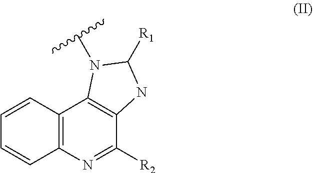

wherein R₁ and R₂ are each independently or may exclude a hydrogen atom, a halogen, an alkyl group, a substituted alkyl group, a heteroalkyl group, a substituted heteroalkyl group, a cycloalkyl group, a substituted cycloalkyl group, a heterocycloalkyl group, a substituted heterocycloalkyl group, an aryl group, a substituted aryl group, a heteroaryl group, a substituted heteroaryl group, an alkoxy group, an alkoxyalkyl group, an alkoxyalkoxy group, and alkoxyalkoxyalkyl group, an amino group, or a hydroxyl group.

Moiety

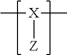

may be further defined as

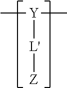

wherein L′ is or may exclude a substituted or unsubstituted alkyl group comprising from 1 to 9 carbon atoms, a substituted or unsubstituted alkyl group comprising from 1 to 9 carbon atoms, or an ethylene glycol moiety comprising from 1 to 9 ethylene glycol groups. L′ may comprise 1, 2, 3, 4, 5, 6, 7, 8, or 9 carbon atoms, or any derivable range therein.

Z may be selected from:

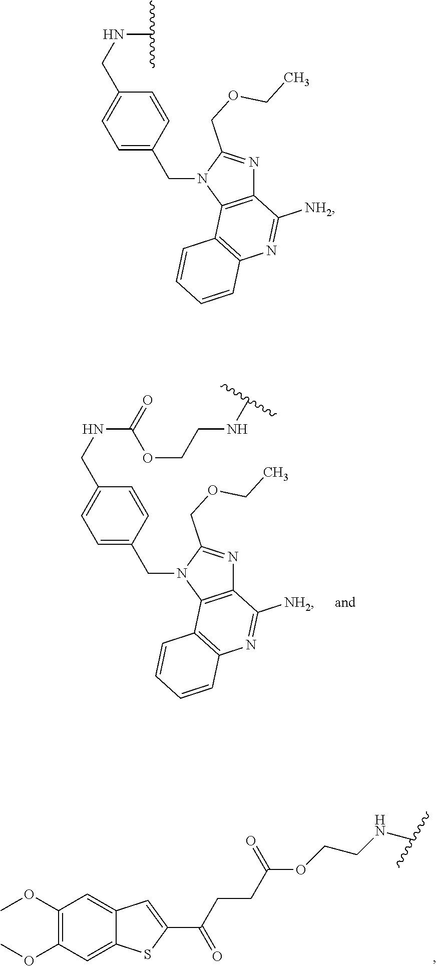 [SMILES: *NCc1ccc(Cn2c(COCC)nc3c(N)nc4ccccc4c32)cc1 ; *NCCOC(=O)NCc1ccc(Cn2c(COCC)nc3c(N)nc4ccccc4c32)cc1 ; *NCCOC(=O)CCC(=O)c1cc2cc(OC)c(OC)cc2s1]

The moiety:

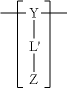

may be

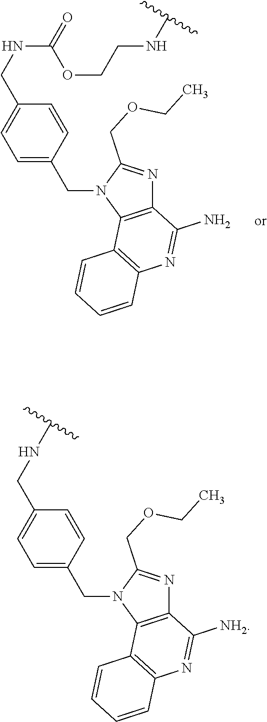 [SMILES: *NCCOC(=O)NCc1ccc(Cn2c(COCC)nc3c(N)nc4ccccc4c32)cc1 ; *NCc1ccc(Cn2c(COCC)nc3c(N)nc4ccccc4c32)cc1]

The moiety:

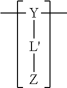

may be

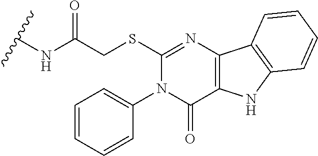 [SMILES: *NC(=O)CSc1nc2c([nH]c3ccccc32)c(=O)n1-c1ccccc1]

The moiety:

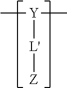

may be

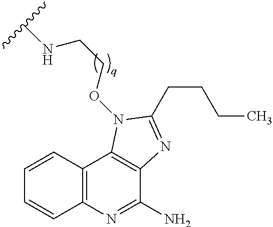

wherein q is 2 to 9. q may include or exclude 2, 3, 4, 5, 6, 7, 8, or 9, or any derivable range therein.

The moiety:

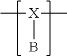

may be further defined as

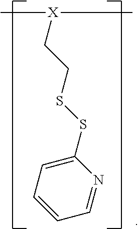

The moiety:

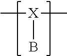

may be further defined as

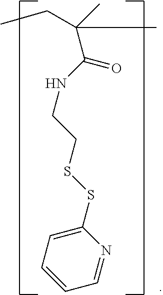

The moiety:

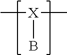

may be further defined as

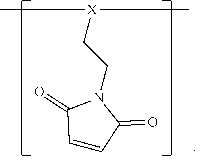

The moiety:

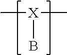

may be further defined as

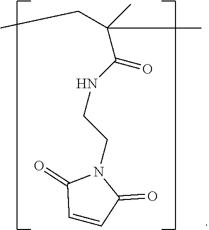

The moiety:

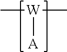

may be further defined as

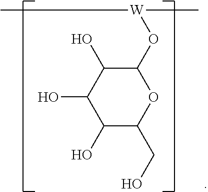

The moiety:

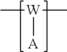

may be further defined as

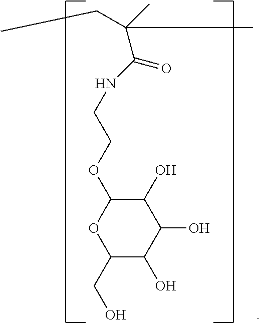

The moiety:

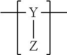

may be further defined as

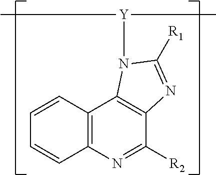

wherein R₁ and R₂ are each independently or may exclude a hydrogen atom, a halogen, an alkyl group, a substituted alkyl group, a heteroalkyl group, a substituted heteroalkyl group, a cycloalkyl group, a substituted cycloalkyl group, a heterocycloalkyl group, a substituted heterocycloalkyl group, an aryl group, a substituted aryl group, a heteroaryl group, a substituted heteroaryl group, an alkoxy group, an alkoxyalkyl group, an alkoxyalkoxy group, and alkoxyalkoxyalkyl group, an amino group, or a hydroxyl group.

The moiety:

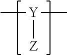

may be further defined as

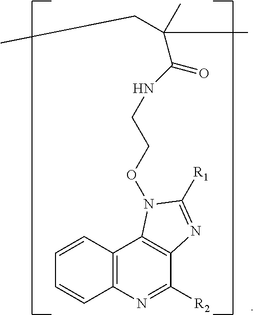

The polymer may comprise at least one TLR or STING agonist and at least one group that binds to an Antigen Presenting Cell (APC) mannose receptor.

The polymer may be further defined as

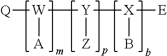

wherein E and Q are end units and each independently comprises or excludes a residue of the polymer, a fluorescent molecule, an albumin polypeptide, a dithioate group, an azide, a linker, an immunomodulating agent, a pyridyl disulfide moiety, a maleimide moiety, a TLR agonist, a STING agonist, a group that binds to an Antigen Presenting Cell (APC) mannose receptor, or combinations thereof.

The dithioate group may be further defined as

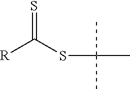

wherein R is an alkyl or aryl group containing from 1 to 12 carbon atoms. R may have 1, 2, 3, 4, 5, 6, 7, 8, 9, 10, 11, or 12 carbon atoms, or any derivable range therein.

The linker may comprise or exclude a polyethylene glycol moiety or a hydrocarbon moiety. The polyethylene glycol linker may comprise from 1 to 150 polyethylene glycol units. The polyethylene glycol linker may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 polyethylene glycol units, or any derivable range therein. The hydrocarbon moiety may comprise from 1 to 150 methylene units. The hydrocarbon moiety may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 methylene units, or any derivable range therein. The polymer may be a copolymer, wherein W, X, and/or Y are different. W, X, and/or Y may be the same. m may be at least 1, p=0, and at least one of Q or E comprises a TLR or STING agonist. The TLR agonist may comprise a CpG TLR agonist. The CpG TLR agonist may comprise ODN1826. The CpG TLR agonist may comprise a phoshorothioated backbone. W, Y, and X may be provided in any order.

The compound may be further defined as

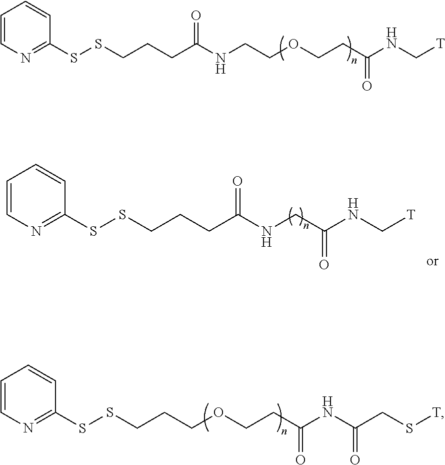

wherein n is from 1 to 150.

The compound may be further defined as

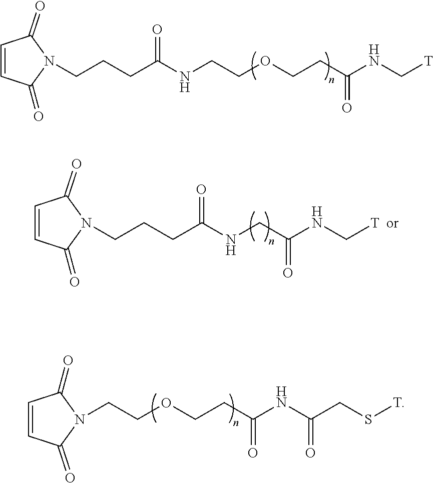

wherein n is from 1 to 150.

The TLR agonist may have the general structure (II),

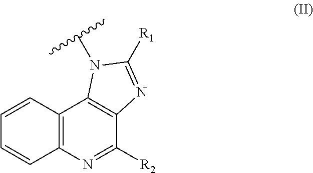

wherein R₁ and R₂ are each independently or exclude a hydrogen atom, a halogen, an alkyl group, a substituted alkyl group, a heteroalkyl group, a substituted heteroalkyl group, a cycloalkyl group, a substituted cycloalkyl group, a heterocycloalkyl group, a substituted heterocycloalkyl group, an aryl group, a substituted aryl group, a heteroaryl group, a substituted heteroaryl group, an alkoxy group, an alkoxyalkyl group, an alkoxyalkoxy group, and alkoxyalkoxyalkyl group, an amino group, or a hydroxyl group.

T may be

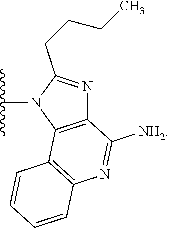 [SMILES: *n1c(CCCC)nc2c(N)nc3ccccc3c21]

T may be

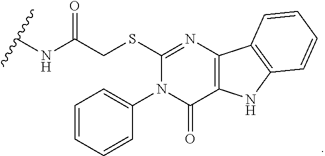 [SMILES: *NC(=O)CSc1nc2c([nH]c3ccccc32)c(=O)n1-c1ccccc1]

T may be

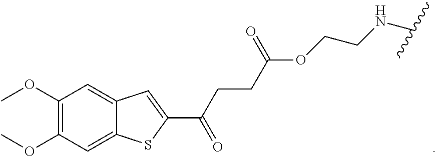 [SMILES: *NCCOC(=O)CCC(=O)c1cc2cc(OC)c(OC)cc2s1]

The polymer or compound may exclude conjugation to a tumor-targeting peptide or further tumor targeting moiety.

The subject may be a human subject. The subject may be a mammal. The subject may be a rat, mouse, pig, horse, dog, cat, rabbit, or cow. The subject may be one that has cancer. The cancer may include or exclude melanoma, a B-cell malignancy, bladder, breast, mammary carcinoma, or colon cancer. The cancer may include or exclude a solid tumor. The B-cell malignancy may include or exclude lymphoma or leukemia. The leukemia may include or exclude acute myeloid leukemia. The B-cell malignancy may include or exclude non-Hodgkin B-cell lymphoma. The non-Hodgkin B-cell lymphoma may be further classified as indolent non-Hodgkin lymphomas, follicular lymphoma, lymphoplasmacytic lymphoma, marginal zone lymphoma, nodal marginal zone lymphoma, gastric mucosa-associated lymphoid tissue (MALT) lymphoma, extragastric MALT lymphoma, mediterranean abdominal lymphoma, splenic marginal zone lymphoma, primary cutaneous anaplastic large cell lymphoma, diffuse large B-cell lymphoma, primary mediastinal B-cell lymphoma, follicular large cell lymphoma, anaplastic large cell lymphoma, cutaneous anaplastic large cell lymphoma, systemic anaplastic large cell lymphoma, extranodal NK-/T-cell lymphoma, lymphomatoid granulomatosis, angioimmunoblastic T-cell lymphoma, peripheral T-cell lymphoma, hepatosplenic T-cell lymphoma, subcutaneous panniculitis-like T-cell lymphoma, enteropathy-type intestinal T-cell lymphoma, intravascular large B-cell lymphoma, Burkitt lymphoma, lymphoblastic lymphoma, adult T-cell leukemia/lymphoma, mantle cell lymphoma, post transplantation lymphoproliferative disorder, true histiocytic lymphoma, primary effusion lymphoma, or plasmablastic lymphoma. The B-cell malignancy may comprise leukemia, and wherein the leukemia is further classified as chronic lymphocytic leukemia, small-lymphocytic leukemia, acute lymphocytic leukemia, acute myeloid leukemia, chronic myeloid leukemia, chronic myelomonocytic leukemia, pediatric leukemia, precursor B acute lymphoblastic leukemia, precursor T acute lymphoblastic leukemia, Burkitt's leukemia, acute biphenotypic leukemia, B-cell prolymphocytic leukemia, acute promyelocytic leukemia, acute myeloblastic leukemia, acute megakaryoblastic leukemia, hairy cell leukemia, T-cell prolymphocytic leukemia, large granular lymphocytic leukemia, adult T-cell leukemia, or clonal eosinophilias.

The method may include or exclude or further comprise administration of one or more additional cancer therapies. The subject may be one that has or will receive an immunotherapy. The method may include or exclude or further comprise administration of an immunotherapy. The immunotherapy may be administered before, after, or concurrent with the compound. The immunotherapy may include or exclude checkpoint inhibitor therapy. The checkpoint inhibitor therapy may include or exclude a PD-1 antibody, a CTLA4 antibody, or both. The immunotherapy may include or exclude a CD40 agonist. The polymer, compound, or composition may be administered systemically. The polymer, compound, or composition may be administered by intravenous injection. The polymer, compound, or composition may be administered intratumorally or peritumorally. The additional therapy may include or exclude one or more of cytarabine, daunorubicin, idarubicin, cladribine, fludarabine, mitoxantrone, etoposide, 6-thioguanine, hydroxyurea, prednisone, dexamethasone, methotrexate, 6-mercaptopurine, azacytidine, and decitabine. The additional therapy may comprise cytarabine.

The additional therapy may be administered before the compound or compositions of the disclosure. The additional therapy may be administered after the compound or composition of the disclosure. The additional therapy may be administered within 12 hours of the compound or composition of the disclosure. The additional therapy may be administered before or after the compound of the disclosure and/or within a time period before or after administration of the compound or composition of the disclosure. The additional therapy, immunotherapy, compound, and/or composition may be administered at a dose of at least once per week for a period of time. The additional therapy, immunotherapy, compound, and/or composition may be administered, administered at least, or administered at most 1, 2, 3, or 4 times per day, 1, 2, 3, 4, 5, 6, or 7 times per week, or 1, 2, 3, 4, 5, 6, 7, 8, 9, 10, 11, 12, 13, 14, 15, 16, 17, 18, 19, or 20 times per month (or any derivable range therein) for a period of time. The period of time may be from 1-12 months. The period of time may be for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks, or 1, 2, 3, 4, or 5 years, or any range derivable therein.

The administered dose of the TLR or STING agonist may be less than the minimum effective dose of the TLR or STING agonist unlinked to the tumor targeting agent. The administered dose of the TLR or STING agonist may be at least 10, 20, 30, 40, 50, 60, 70, 80, or 90% (or any derivable range therein) less than the minimum effective dose of the TLR or STING agonist unlinked to the tumor targeting agent. The administered dose of the TLR or STING agonist may be less than the minimum effective dose of the TLR or STING agonist unlinked to the tumor targeting agent linked to the albumin polypeptide. The administered dose of the TLR or STING agonist may be at least 10, 20, 30, 40, 50, 60, 70, 80, or 90% (or any derivable range therein) less than the minimum effective dose of the TLR or STING agonist unlinked to the tumor targeting agent and albumin polypeptide. The subject may be one that has been previously treated with an adjuvant. The subject may be one that has been determined to be non-responsive to the previous treatment or wherein the wherein the subject experienced non-specific toxicity to the previous treatment. The subject may be one that has experienced greater than 2, 3, 4, or 5 immune related adverse events in response to the prior therapy.

The terms “protein”, “polypeptide” and “peptide” are used interchangeably herein when referring to a gene product.

The terms “subject,” “mammal,” and “patient” are used interchangeably. The subject may be a mammal. The subject may be a human. The subject may be a mouse, rat, rabbit, dog, donkey, or a laboratory test animal such as fruit fly, zebrafish, etc.

It is contemplated that the methods and compositions include exclusion of any of the aspects described herein.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one.” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z.” “(x and y) or z.” “x or (y and z),” or “x or y or z.” It is specifically contemplated that x, y, or z may be specifically excluded from an embodiment or aspect.

The words “comprising” (and any form of comprising, such as “comprise” and “comprises”). “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise.” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments or aspects described in the context of the term “comprising” may also be implemented in the context of the term “consisting of” or “consisting essentially of.”

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments or aspects discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments or aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Structure of representative PDS containing polymer. 1H NMR spectra obtained on Bruker Avance-II 400 MHz and analyzed with MnovaNMR (Mestrelab).

FIG. 2. Gel electrophoresis confirms CPG-FAM-p(Man-PDS-HPMA) conjugation and purity. Lane (a) contains free amine-CPG-FAM. Lane (b) contains completed reaction between amine-CPG-FAM and DBCO-NHS. Lane (c) contains crude reaction mixture of CPG-FAM-p(Man-PDS-HPMA) with remaining amine-CPG-FAM. Lane (d) contains purified CPG-FAM-p(Man-PDS-HPMA). Chemidoc MP imaging system (Bio-Rad) was used for imaging of polyacrylamide gel.

FIG. 3. CPG-FAM-p(Man-PDS-HPMA) stimulate Raw 264.7 Reporter Macrophages on par with free CpG. RAW-Blue Mouse Macrophage Reporter Cells (InvivoGen) were incubated with CPG-FAM-p(Man-PDS-HPMA) or free CpG for 20 hours. NF-κB/AP-1 secretion was then quantified via spectrophotometry by the addition of Quantiblue (InvivoGen), per manufacturer's instructions.

FIG. 4A-4B. PDS containing polymers bind tumor cells in vitro. Flow cytometric analysis of dye-labeled (A) PDS containing polymer binding to B16F10 cells or (B) PDS and/or mannose (Man) containing polymer binding to EMT6 cells in vitro after a 90-minute incubation on ice to prevent endocytosis.

FIG. 5. PDS containing polymers bind tumor cells in vivo. Flow cytometric analysis of dye-labeled polymer binding to cells in the tumor. Respective frequency values are given as a percent of live singlet cells that are positive for immune cell marker CD45 or tumor cell marker TRP1.

FIG. 6A-6D. p(Man-TLR7-PDS) binding reduces upregulation of systemic pro-inflammatory cytokines relative to a non-binding p(Man-TLR7) control. 500,000 B16F10 cells were inoculated intradermally on day 0 into the left shoulder of 8 week old female C57BL/6 mice. Mice were injected intratumorally with 40 μg TLR7 equivalent polymer (or vehicle control PBS) on days 6 and 9. Mice were bleed via submandibular bleed and plasma was analyzed for pro-inflammatory cytokines. Relative amounts of (A) Interferon gamma, (B) Tumor Necrosis Factor alpha, (C) Interleukin-12, and (D) Interleukin-6 are shown, grouped by treatment.

FIG. 7A-7B. p(Man-TLR7-PDS) does not cause weight loss and significantly prolongs survival in B16F10 melanoma. 500,000 B16F10 cells were inoculated intradermally on day 0 into the left shoulder of 8 week old female C57BL/6 mice. Mice were injected intratumorally with 10, 20, or 40 μg TLR7 equivalent polymer (or vehicle control PBS) on days 6, 9, and 12. (A) Full survival analysis. Statistical analysis was determined using Log-rank (Mantle-Cox) test. *p<0.05. (B) Fold change in body weights since the start of treatment on day 6, grouped by treatment. On both panels, arrows denote treatment days.

FIG. 8A-8B. PDS containing p(Man-TLR7) slows tumor growth and prolongs survival in B16F10 melanoma. 500,000 B16F10 cells were inoculated intradermally on day 0 into the left shoulder of 8 week old female C57BL/6 mice. Mice were injected intratumorally with 40 μg TLR7 equivalent polymer (or vehicle control PBS) on days 6, 9, and 12. (A) Individual tumor growth curves by treatment and (B) full survival analysis. Statistical analysis for survival curve was determined using Log-rank (Mantle-Cox) test. *p<0.05. Arrows denote treatment days.

FIG. 9A-9B. p(Man-TLR7-PDS) in combination with anti-PD-1 slows tumor growth and prolongs survival in CT26 colon carcinoma. 500,000 CT26 cells were inoculated subcutaneously on day 0 into the left shoulder of 8 week old Balb/c mice. Mice were injected intratumorally with 40 μg TLR7 equivalent polymer (or vehicle control PBS) and intraperitoneally with 100 μg anti-PD-1 on days 6, 9, 12, and 15. (A) Grouped tumor growth curves #1/8 mouse died without significant tumor growth. For all panels, arrows denote treatment days. (B) Full survival analysis. Statistical analysis for survival curve was determined using Log-rank (Mantle-Cox) test. *p<0.05, **p<0.01. ***p<0.001. For both panels, arrows denote treatment days.

FIG. 10A-10G. Polymeric PDS binds tumor cells in vitro. (A) Schematic of PDS disulfide exchange with exofacial protein thiols on tumor cells, enabling in situ adjuvant conjugation. (B) Mean fluorescence intensity (MFI) data of concentration-dependent binding of fluorescently labeled p(PDS) or “spacer” only p(HPMA) to B16F10 cells as quantified by flow cytometry. The same assay was repeated on (C) EMT6, (D) CT26, (E) MC38, and (F) MDA-MB-231 cells (mean+/−SEM; n=3). (G) Fold change in binding (relative to untreated cells) of fluorescently labeled p(PDS) to B16F10 cells after pre-blocking exofacial protein thiols with N-ethyl maleimide or pre-reducing extracellular disulfide bonds with TCEP (mean+/−SEM; n=4). Statistical analysis was done using ordinary one-way analysis of variance. p<0.001.

FIG. 11A-11E. PDS polymers preferentially bind tumor cells in vivo. Mice bearing established B16F10 tumors were injected intratumorally with fluorescently labeled p(PDS) or p(HPMA). After 3 hr. tumors were digested and stained for flow cytometry. (A) Representative contour flow cytometry plots, comparing the frequency of polymer⁺ cells of CD45⁺ immune cells or TRP1⁺ tumor cells between mice treated with p(PDS) in red or p(HPMA) in blue. Both polymers with (B) low or (C) high PDS content preferentially bound TRP1⁺ tumor cells over CD45⁺ immune cells, as quantified by polymer median fluorescence intensity (MFI) of the parent population. (D) Both PDS-containing polymers bound a higher frequency of TRP1⁺ tumor cells than the non-binding p(HPMA) control (mean+/−SEM; n=5). Experiment was repeated twice with similar results. Representative data shown. (E) Alternatively, after 6 hours, tumors were harvested and analyzed by fluorescence microscopy. Tissues were stained with DAPI (grey) and AF594 conjugated anti-thioredoxin-1 (red). Merged panel contains only thioredoxin-1 and polymer (blue) channels. Scale bars. 100 μm. Representative images of n=2 biological replicates with n=3 technical replicates. Statistical analyses were performed using unpaired t-tests (B to C) and ordinary one-way analysis of variance (D). **p<0.01; ***p<0.001; ****p<0.0001; ns=not significant.

FIG. 12A-12I. Synthesis and characterization of p(Man-TLR7-PDS). (A) Structure of full p(Man-TLR7-PDS) statistical co-polymer. (B) GPC elution profile of p(Man-TLR7-PDS) and non-binding control p(Man-TLR7). (C) Table of monomer feed ratios relative to chain transfer agent used to synthesize p(Man-TLR7-PDS) and p(Man-TLR7) and weight percent TLR7 monomer of resulting polymers. (D) Mice bearing established B16F10 tumors were injected intratumorally with fluorescently conjugated p(Man-TLR7-PDS) or p(Man-TLR7). After 3 hr. tumors were digested and stained for flow cytometry. Full p(Man-TLR7-PDS) preferentially binds TRP1⁺ tumor cells over CD45⁺ immune cells, as quantified by polymer median fluorescence intensity (MFI). Non-binding p(Man-TLR7) showed significantly less binding to tumor cells than p(Man-TLR7-PDS). (E to I) Concentration-dependent secretion of pro-inflammatory cytokines by RAW 264.7 cells in response to stimulation with p(Man-TLR7-PDS) including (E) IL-6, (F) MCP-1, (G) TNFα, (H) IL-27, or (I) IL-1a with control TLR7/8 agonist R848 (mean+/−SEM; n=3). Statistical analyses were performed using unpaired t-tests. **p<0.01; ***p<0.001; ns=not significant.

FIG. 13A-13C. p(Man-TLR7-PDS) significantly slows tumor growth and prolongs survival in various murine cancer models. (A) Mice were inoculated intradermally on the left shoulder with 500,000 B16F10 melanoma cells and treated intratumorally with 40 μg TLR7 monomer equivalent p(Man-TLR7-PDS) (n=13), p(Man-TLR7) (n=12), or vehicle control (PBS, n=10) on days 6, 9, and 12 after tumor inoculation. Data are compiled from two independent experiments. (B) Mice were inoculated with 500,000 EMT6 mammary carcinoma cells into the left mammary fat pad and were treated intratumorally with 40 μg TLR7 monomer equivalent p(Man-TLR7-PDS) (n=9) or non-binding p(Man-TLR7) (n=8) or vehicle control (PBS, n=9) on days 6, 9, 12, and 15 after tumor inoculation. (C) Mice were inoculated subcutaneously on the left shoulder with 500,000 CT26 colon carcinoma cells and treated with PBS (n=7), 100 μg anti-PD-1 (n=7), 40 μg TLR7 monomer equivalent polymer (n=8), or polymer+anti-PD-1 (n=8) on days 6, 9, 12, and 15 after tumor inoculation. Polymer or PBS were administered intratumorally and anti-PD-1 was administered intraperitoneally. For all experiments, shown are individual (thin dotted lines) and mean (thick solid lines) tumor growth curves and survival plots. Arrows denote treatment days on survival plots. Statistical analyses were performed using unpaired 1-tests of tumor measurements on the last day all mice are surviving and log-rank (Mantel-Cox) curve comparison for survival.

FIG. 14A-14G. p(Man-TLR7-PDS) effectively eradicates MC38 colon carcinoma and limits toxicity of TLR7/8 agonist therapeutics. (A) Mice were inoculated subcutaneously with 500,000 MC38 colon carcinoma cells into the left shoulder and injected with 20 or 40 μg TLR7 monomer equivalent polymer on days 7, 10, and 13 after tumor inoculation. Shown are individual (thin dotted lines) and mean (thick solid lines) tumor growth curves and survival plots. (B) Mice were inoculated subcutaneously with 500,000 MC38 colon carcinoma cells into the left shoulder. CD4, CD8, CSF1R, or isotype control depletion antibodies were administered as described in methods. Mice were treated intratumorally with 40 μg TLR7 monomer equivalent polymer or vehicle control (PBS) every three days for a total of three injections once tumors reached a volume of 100 mm³ (n=7 for all groups). Shown are individual (thin dotted lines) and mean (thick solid lines) tumor growth curves and survival plots. (C to G) MC38 tumor-bearing mice were injected intratumorally with p(Man-TLR7-PDS), control TLR7/8 agonist 3M-052, or PBS. 6 hours after injection, sera were collected and analyzed for pro-inflammatory cytokines, including (C) IFNγ, (D) IL-6, (E) MCP-1, (F) IFNβ, and (G) IL-27. Results were validated using a similar experimental design in the B16F10 melanoma model with similar results. Representative data shown. Statistical analyses were performed using unpaired 1-tests of tumor measurements on the last day all mice are surviving (A), one way analysis of variance on the last day all mice are surviving (B), log-rank (Mantel-Cox) curve comparison for survival (A to B), and ordinary one-way analysis of variance (C to G). *p<0.05 **p<0.01; ***p<0.001, ****p<0.0001.

FIG. 15. Thiol-reactive polymeric adjuvant covalently binds unpaired cysteines upon intratumoral injection to adjuvant tumor cells and debris for immune recognition and subsequent slowed tumor growth.

FIG. 16. GPC elution profiles of all synthesized polymers. Gel permeation chromatography was performed using Tosoh EcoSEC size exclusion chromatography system with Tosoh SuperAW3000+Tosoh SuperAW4000 columns, eluted in DMF+0.01 M LiBr at 50° C. Full elution profile and magnification of relevant region shown, along with the PMMA calibration standards.

FIG. 17. Extent of PDS reduction can be monitored with UV-Vis Spectrophotometry. p(PDS) was dissolved in PBS and the full UV-Vis absorbance spectrum was recorded. Reducing agent β-mercaptoethanol was added in 100× excess of PDS and allowed to react to completion (30 minutes). This verifies that the PDS groups are functionally active and can be used to quantify PDS content using a standard curve of 2-mercaptopyridine.

FIG. 18. Gating strategy for in vivo binding of PDS polymers. Cells were collected on BS LSRFortessa and data were analyzed in FlowJo. Cells were gated on FSC-A vs. SSC-A, single cells were gates on FSC-A vs. FSC-H. and live cells were gated on FSC-H vs. BV-421. From there. CD45⁺ cells were gated on PE-A vs. FSC-H. Alternatively. TRP1⁺ cells were gated on FITC-A vs. FSC-H. The positive populations from each of those gates was gated for Polymer⁺ cells on APC-A vs. FSC-H. Alternatively, the APC MFI of the whole TRP1⁺ or CD45⁺ populations was recorded.

FIG. 19. Intracellular thioredoxin-1 is detectable in B16F10 cells via flow cytometry. B16F10 cells were removed from culture. washed free of media with PBS two times, then permeabilized using FIX&PERM Cell Permeabilization Kit (ThermoFisher) for intracellular antibody staining with AF594-conjugated anti-thioredoxin-1 (Novus Biologicals). Cells were collected by flow cytometry as described in methods. Representative histogram of staining shown.

FIG. 20. Polymer binding to tumor cells is not mannose dependent. EMT6 cells were removed from culture, washed free of media with PBS two times, then incubated on ice with various concentrations of AZDye 674-labeled polymer for 90 minutes (n=3, mean+/−SEM). Polymers had similar numbers of mannose monomer or PDS monomer per chain as quantified by proton NMR and were molecular weight matched. Cells were collected by flow cytometry as described in methods, where MFI correlates to amount of polymer attached to the cells. Polymers containing PDS monomers showed concentration dependent binding to tumor cells, regardless of the incorporation of mannose monomer. Likewise, polymers without PDS did not show significant binding to tumor cells, regardless of mannose monomer. Statistical analyses were performed with ordinary one-way analysis of variance with Tukey's test.

FIG. 21. PDS polymer binding to tumor cells is not altered by mannose pre-incubation. B16F10 cells were removed from culture, washed free of media with PBS two times, then incubated with 1 mg/mL D-mannose on ice for 30 minutes. AZDye 674-labeled polymer was added directly to the mannose mixture to make a polymer solution at 80 μM fluorophore then incubated for an additional 90 minutes (n=3, mean+/−SEM). Cells were collected by flow cytometry as described in methods, where MFI correlates to amount of polymer attached to the cells. Binding is confirmed to be independent of mannose pre-incubation, as the polymer MFI does not change. Statistical differences were determined by unpaired t-tests.

FIG. 22. p(Man-TLR7-PDS) stimulates murine BMDCs. Murine bone marrow-derived dendritic cells (BMDCs) were treated with various concentrations of p(Man-TLR7-PDS) or p(Man-TLR7) on a TLR7 monomer basis. Supernatant IL-6 was measured after 6 hours via LEGENDplex (n=3, mean+/−SEM). Results show that both the binding and non-binding polymer variants can stimulate dendritic to produce pro-inflammatory cytokines.

FIG. 23. Polymer uptake by BMDCs is mediated by mannose. Murine bone marrow-derived dendritic cells (BMDCs) were incubated with various concentrations of AZ647 dye-labelled polymer in complete Lutz media for 45 minutes. The cells were washed and fixed with 2% paraformaldehyde in PBS for flow cytometric analysis. Cells were collected by flow cytometry as described in methods, where MFI correlates to amount of polymer taken up by the cells. Uptake is confirmed to be dependent on the incorporation of mannose and is not significantly affected by PDS-mediated binding. Statistical differences were determined by unpaired t-tests.

FIG. 24. p(Man-TLR7-PDS) is not inherently cytotoxic to RAW 264.7 macrophages. RAW 264.7 macrophage-like cells were purchased from ATCC and cultured according to instructions. One day after plating in a flat-bottom, non-treated 96 well plate, cells were treated with various concentrations of TLR7 equivalent polymer (as quantified by absorbance at 327 nm). 24 hours after treatment, cells were stained with Zombie Aqua Fixable Viability Dye (BioLegend) and collected by flow cytometry as described in methods. Live cells as a percent of total cells is shown at various polymer or control TLR7/8 agonist R848 (Sigma), with no significant change from the media alone control wells.

FIG. 25. p(Man-TLR7-PDS) is not inherently cytotoxic to B16F10 melanoma cells. Cells were seeded at a density of 8.000 cells per well and incubated overnight with various concentrations of p(Man-TLR7-PDS). The following day, the cells were analyzed using MTT Cell Viability Assay (ThermoFisher) according to manufacturer's protocol. Even at high concentrations of polymer, high cell viability was observed.

FIG. 26. p(Man-PDS) has no inherent antitumor efficacy. 8-week-old female C57BL/6 mice were inoculated with MC38 tumors as described in methods. On days 7, 10, and 13 postinoculation, mice were injected intratumorally with 260 μg p(Man-PDS), which corresponds by total mass to our typical dose of 40 μg TLR7 monomer (n=6, mean+/−SEM). The volume of the tumor was recorded as previously described. Mice were euthanized when the tumor volume exceeded 500 mm³ and/or based on humane end-point criteria. Here we demonstrate that the non-adjuvanted p(Man-PDS) has no inherent antitumor efficacy. Statistical differences were determined by pairwise log-rank (Mantel-Cox) tests.

FIG. 27, p(Man-TLR7-PDS) has low batch-to-batch variability. Two batches of p(Man-TLR7-PDS). AS146 and AS122-2, were synthesized using separate batches of CTA, mannose monomer, PDS monomer, and TLR7 monomer. 8-week-old female C57BL/6 mice were inoculated with B16F10 tumors as described in methods. On days 6, 9, and 12 post-inoculation, mice were injected intratumorally with 40 μg TLR7 monomer equivalent of AS146 or AS122-2 (n=7) or vehicle control (PBS, n=6). The volume of the tumor was recorded as previously described. Mice were euthanized when the tumor volume exceeded 500 mm³ and/or based on humane end-point criteria. Here we verify the low variability of batches of p(Man-TLR7-PDS).

FIG. 28. Dose dependent effect of p(Man-TLR7-PDS) in B16F10 melanoma. 8-week-old female C57BL/6 mice were inoculated with B16F10 tumors as described in methods. On days 6, 9, and 12 post-inoculation, mice were injected intratumorally with various concentrations of polymer (n=7) or vehicle control (PBS, n=5) (mean+/−SEM). The volume of the tumor was recorded as previously described. Mice were euthanized when the tumor volume exceeded 500 mm³ and/or based on humane end-point criteria. Here we demonstrate that the polymer has dose-dependent antitumor efficacy and significantly prolongs survival of tumor-bearing mice. Statistical differences were determined by pairwise log-rank (Mantel-Cox) tests. *p<0.05.

FIG. 29. p(Man-TLR7-PDS) does not cause weight loss in healthy mice. Healthy 8-week-old female C57BL/6 mice were injected subcutaneously three times three days apart with various concentrations of polymer in sterile PBS (or vehicle control) (n=5, mean+/−SEM). Doses were defined by TLR7 monomer content as quantified by absorbance at 327 nm, based on a standard curve of monomer. Mouse weight was recorded on each injection day and two days after the final injection. At all doses and timepoints, mice did not, on average, lose a significant portion of body weight.

FIG. 30. p(Man-TLR7-PDS) does not cause weight loss in tumor-bearing BALB/c mice. 8 week-old female BALB/c mice were inoculated with CT26 tumors as described in methods. On days 6, 9, and 12 post-inoculation, mice were injected intratumorally with 40 μg TLR7 monomer equivalent p(Man-TLR7-PDS) (n=8) or vehicle control (PBS, n=7) (mean+/−SEM). The mouse weights were recorded every three days. Mice were euthanized when the tumor volume exceeded 500 mm³ and/or based on humane end-point criteria.

FIG. 31. p(Man-TLR7-PDS) does not cause weight loss in tumor-bearing C57BL/6 mice. 8 week-old female C57BL/6 mice were inoculated with B16F10 tumors as described in methods. On days 6, 9, and 12 post-inoculation, mice were injected intratumorally with various concentrations of p(Man-TLR7-PDS) (n=7, mean+/−SEM). The mouse weights were recorded every three days. Mice were euthanized when the tumor volume exceeded 500 mm³ and/or based on humane end-point criteria.

FIG. 32. Vehicle for 3M-052 injection does not produce systemic cytokines compared to PBS. 8-week-old female C57BL/6 mice were inoculated with MC38 tumors as described in methods. Mice were injected intratumorally with 30 μL PBS (n=8) or vehicle control consisting of 10% DMSO 40% PEG300, and 5% Tween-80 in PBS (n=3). Six hours after injection, sera was collected and analyzed for proinflammatory cytokines, including INFγ, IL-6, MCP-1, IFNβ, and IL-27 (mean+/−SEM). Statistical analyses were performed using Mann-Whitney tests for nonparametric data.

FIG. 33. Intratumorally injected p(Man-TLR7-PDS) does not induce upregulation of systemic proinflammatory cytokines. 8-week-old female C57BL/6 mice were inoculated with B16F10 tumors as described in methods. On days 6 and 9 post-inoculation, mice were injected intratumorally with 40 μg TLR.7 monomer equivalent p(Man-TLR7-PDS) or p(Man-TLR7) or vehicle control (PBS). On day 11, sera was collected and analyzed for proinflammatory cytokines, including INFγ, TNFα, IL-12p70, and IL-6 (n=7 for all groups, mean+/−SEM). Statistical analyses were performed using Kruskal-Wallis tests followed by Dunn's multiple comparison for nonparametric data. All differences are non-significant.

FIG. 34. p(Man-TLR7-PDS) has significant antitumor efficacy in ectopic EMT6. 8-week-old female BALB/c mice were inoculated with 500.000 EMT6 mammary carcinoma cells subcutaneously into the shaved left shoulder. On days 6, 9, and 12 post-inoculation, mice were injected intratumorally with 40 μg TLR7 monomer equivalent p(Man-TLR7-PDS) (n=8) or vehicle control (PBS, n=7). The volume of the tumor was recorded as previously described. Mice were euthanized when the tumor volume exceeded 500 mm³ and/or based on humane endpoint criteria. Shown are individual (thin dotted lines) and mean (thick solid lines) tumor growth curves and survival plot. Arrows denote treatment days. Statistical differences were determined by pairwise log-rank (Mantel-Cox) tests.

FIG. 35A-35C. PDS-containing polymers bind C1498 cells in vitro and binding is altered by cytarabine pre-treatment. Flow cytometric analysis of dye labeled PDS-containing polymers binding to C1498 cells in a (A) polymer dose-dependent manner. Binding efficiency is also modulated by (B) dose of cytarabine pre-treatment and (C) duration of cytarabine pre-treatment.

FIG. 36A-36B. p(PDS-HPMA) binds dead cells and C1498 cells most efficiently. Flow cytometric analysis of dye-labeled polymer binding to white blood cells ex vivo. Polymer binds preferentially to (A) C1498 cancer cells over other healthy immune cells and (B) dead cells over live cells.

FIG. 37. p(Man-TLR7-PDS) accumulates primarily in the liver. Fluorescence quantification of tissue homogenate from C1498 mice after injection of dye-labeled p(Man-TLR7-PDS).

FIG. 38A-38B. p(Man-TLR7-PDS) is well tolerated and synergizes with cytarabine to prolong survival of AML bearing mice. 1,000,000 C1498 cells were inoculated intravenously on day 0. Mice were injected intraperitoneally with 20 mg cytarabine on days 1, 8, 13, and 20 and intravenously with 40 μg TLR7 equivalent p(Man-TLR7-PDS) (as quantified by absorbance at 327 nm) on days 2, 9, 14, and 21. (A) Survival plot and (B) fold change in weight for duration of survival across experiment are shown.

FIG. 39A-39B. Full therapeutic efficacy requires both adjuvant and chemotherapy but if effective with only one dose. 1,000,000 C1498 cells were inoculated intravenously on day 0. (A) Mice were injected intraperitoneally with 20 mg cytarabine on days 1, 8, 13, and 20 and intravenously with 40 μg TLR7 equivalent p(Man-TLR7-PDS) (as quantified by absorbance at 327 nm) or weight equivalent p(Man-PDS) on days 2, 9, 14, and 21. (B) Mice were injected intraperitoneally with 20 mg cytarabine on day 2 and intravenously with 40 μg TLR7 equivalent p(Man-TLR7-PDS) (as quantified by absorbance at 327 nm) six hours after cytarabine treatment on day 2. Survival plots for both are shown.

FIGS. 40A-40C. T and B cell responses are involved in p(Man-TLR7-PDS) treatment response. (A) Median fluorescence intensity by flow cytometry of endogenous anti-C1498 cell antibodies as detected by secondary anti-IgG antibody. (B-C) Frequencies of CD8 T cell subtypes in the spleen following polymer treatment including (B) CD137⁺ and (C) PD-1⁺.

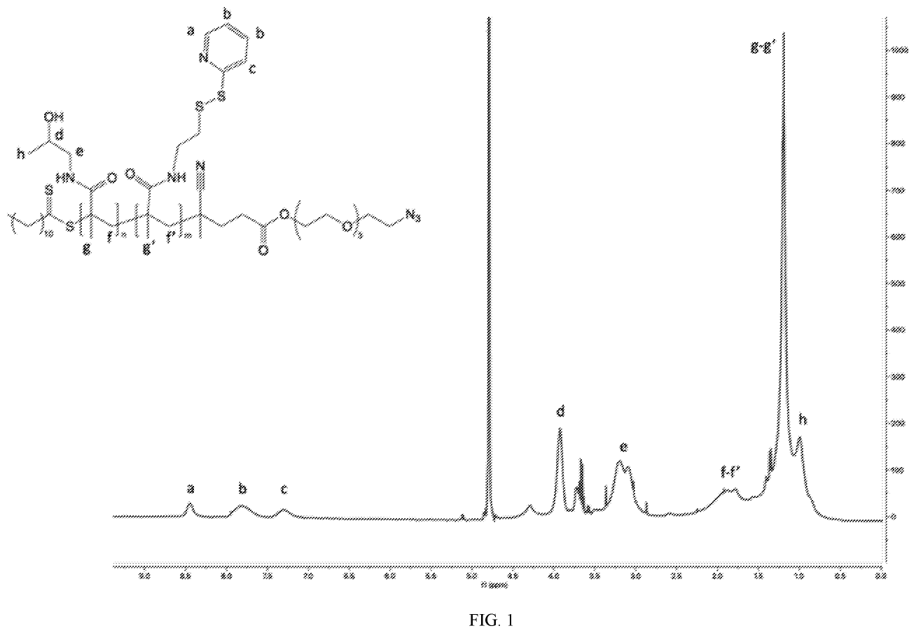

### DETAILED DESCRIPTION

While solid tumors vary drastically, some physiological properties are common and create a characteristic microenvironment for cancer cell growth and proliferation which includes hypoxia and acidosis (8, 9). Consequently, the inventors hypothesized that the metabolic stress of tumor growth also produces an excess of unpaired cysteines on cell surface proteins relative to the rest of the body. To the inventor's knowledge, this phenomenon has not yet to be directly exploited for a drug delivery application. Here, the inventors investigate the use of macromolecular, multivalent thiol-reactive polymers that bear one or multiple immune agonist molecules, such as TLR7/8, TLR9, TLR4, or TLR2/6 agonists. Further, these polymers can be functionalized with moieties to enhance uptake by antigen-presenting cells such as dendritic cells, for example mannose moieties.

The inventors hypothesized that Pyridyl disulfide (PDS) moieties could be used to bind free thiols in situ. Using PDS chemistry, they developed a fully synthetic platform to deliver mono- or multivalent adjuvants to endosomal TLRs, including TLR7/8 and TLR9 agonists, or cell surface TLRs, including TLR4 and TLR2/6. These TLR agonists have both been reported to have strong anticancer efficacy, both alone and in combination (29-36). They hypothesize that this delivery platform can increase efficacy of these therapeutics, by associating them closely with tumor cells and tumor cell debris. Further, they hypothesize that this delivery platform can increase the therapeutic index, by localizing the immune agonist molecule to the tumor and tumor draining lymph nodes, reducing leakage into the systemic circulation where side-effects such as cytokine storm may ensue. In a specific implementation, the resulting methacrylamide polymer consists of PDS monomers to bind tumor cell surfaces, mannose monomers to trigger internalization by antigen presenting cells, 2-hydroxypropyl methacrylamide as a water soluble “spacer”, and either a TLR7 agonist monomer or a chain end conjugated TLR9 agonist, CpG.

### I. DEFINITIONS

The term “each independently” is used herein to indicate that the choices can be identical or different, i.e., in the case of R groups, for example, the term “each independently” indicates that the R groups (e.g., R1, R2) can be identical (e.g., R1 and R2 may both be substituted alkyl groups) or different (e.g., R1 may be an alkyl group and R2 may be an alkoxy group) specified otherwise, a named R group will have the structure recognized in the art as corresponding to R groups with that name. For the purposes of illustration, representative R groups are defined herein. These definitions are intended to supplement and illustrate, not preclude, the definitions known to those of skill in the art.

The term “aliphatic group” denotes an acyclic or cyclic, saturated or unsaturated hydrocarbon group excluding aromatic compounds. “Substituted aliphatic group” refers to an aliphatic group as just described in which one or more hydrogen atoms attached to carbon of the aliphatic group is replaced by any other group, such as halogen, aryl, substituted aryl, heteroaryl, substituted heteroaryl, cycloalkyl, alkoxy, amino, ester, amide, alcohol, and combinations thereof.

The term “alkyl group” denotes a monovalent linear or branched saturated hydrocarbon group of 1 to 12 carbon atoms. An alkyl group may have 1 to 7 carbon atoms. An alkyl group may have 1 to 4 carbon atoms. Non-limiting examples of alkyl groups include methyl, ethyl, propyl, isopropyl, n-butyl, iso-butyl, and sec-butyl. Particular alkyl groups include methyl, ethyl, propyl and isopropyl. More particular alkyl groups are methyl, ethyl and propyl.

The term “substituted alkyl group” refers to an alkyl group as just described in which one or more hydrogen atoms attached to at least one carbon of the alkyl group is replaced by any other group, such as halogen, aryl, substituted aryl, heteroaryl, substituted heteroaryl, cycloalkyl, alkoxy, amino, ester, amide, alcohol, and combinations thereof.

The term “cycloalkyl group” denotes a cyclized alkyl group, for example cyclopropyl, cyclobutyl, cyclopentyl, cyclohexyl, etc.

The term “substituted cycloalkyl group” refers to a cycloalkyl group as just described in which one or more hydrogen atoms attached to at least one carbon of the cycloalkyl group is replaced by another group, such as halogen, alkyl, substituted alkyl, aryl, substituted aryl, cycloalkyl, substituted cycloalkyl, heterocycloalkyl, heteroaryl, substituted heteroaryl, alkoxy, aryloxy, boryl, phosphino, amino, thio, ester, amide, alcohol and combinations thereof.

The term “heteroalkyl group” refers to an alkyl or a substituted alkyl group as described above in which one or more carbon atoms are replaced with a heteroatom from the group consisting of N, O, P, B, S, Si, Se and Ge. The bond between the carbon atom and the heteroatom may be saturated or unsaturated. Examples include an alkoxy group such as methoxy, ethoxy, propoxy, iso-propoxy, butoxy or t-butoxy, an alkoxyalkyl group such as methoxymethyl, ethoxymethyl, 1-methoxyethyl, 1-ethoxyethyl, 2-methoxyethyl or 2-ethoxyethyl, an alkylamino group such as methylamino, ethylamino, propylamino, isopropylamino, dimethylamino or diethylamino, an alkylthio group such as methylthio, ethylthio or isopropylthio or a cyano group. Thus, an alkyl group substituted with a group such as heterocycloalkyl, substituted heterocycloalkyl, heteroaryl, substituted heteroaryl, alkoxy, aryloxy, boryl, phosphino, amino, imino, or thio is within the scope of the term heteroalkyl group.

The term “heterocycloalkyl group” refers to a cycloalkyl group as described, but in which one or more or all carbon atoms of the unsaturated group are replaced by a heteroatom from the group consisting of N, O, P, B, S, Si, Se and Ge. Suitable heterocycloalkyl groups include, for example, piperazinyl, morpholinyl, tetrahydropyranyl, tetrahydrofuranyl, piperidinyl and pyrrolidinyl.

The term “substituted heterocycloalkyl group” refers to a heterocycloalkyl group as just described, but in which one or more hydrogen atoms on any atom of the heterocycloalkyl group is replaced by another group such as a halogen, alkyl, substituted alkyl, aryl, substituted aryl, cycloalkyl, substituted cycloalkyl, heterocycloalkyl, heteroaryl, substituted heteroaryl, alkoxy, aryloxy, boryl, phosphino, amino, thio, and combinations thereof.

The term “aryl group” refers to an aromatic substituent, which may be a single aromatic ring or multiple aromatic rings which are fused together, linked covalently, or linked to a common group such as a methylene or ethylene moiety. The common linking group may also be a carbonyl as in benzophenone or a heteroatom, such as oxygen in the case of diphenylether or nitrogen in the case of diphenylamine. The aromatic ring(s) may include phenyl, naphthyl, biphenyl, diphenylether, diphenylamine and benzophenone among others. Aryl groups may have between 1 and 50 carbon atoms, 1 and 9 carbon atoms, or 1 and 6 carbon atoms.

The term “substituted aryl group” refers to an aryl group as just described in which one or more hydrogen atoms attached to any carbon atom is replaced by one or more functional groups such as alkyl, substituted alkyl, cycloalkyl, substituted cycloalkyl, heterocycloalkyl, substituted heterocycloalkyl, halogen, halogenated alkyl (e.g., CF3), hydroxy, amino, phosphino, alkoxy, amino, thio and both saturated and unsaturated cyclic hydrocarbons which are fused to the aromatic ring(s), linked covalently or linked to a common group such as a methylene or ethylene moiety. The linking group may also be a carbonyl such as in cyclohexyl phenyl ketone. Specific example of substituted aryl groups include perfluorophenyl, chlorophenyl, 3,5-dimethylphenyl, 2,6-diisopropylphenyl and the like.

The term “heteroaryl group” refers to aromatic ring(s) in which one or more carbon atoms of the aromatic ring(s) are replaced by a heteroatom(s) such as nitrogen, oxygen, boron, selenium, phosphorus, silicon or sulfur. Heteroaryl group refers to structures that may be a single aromatic ring, multiple aromatic ring(s), or one or more aromatic rings coupled to one or more nonaromatic ring(s). In structures having multiple rings, the rings can be fused together, linked covalently, or linked to a common group such as a methylene or ethylene moiety. The common linking group may also be a carbonyl as in phenyl pyridyl ketone. Rings such as thiophene, pyridine, oxazole, isoxazole, thiazole, isothiazole, isophthalimide, pyrazole, indole, pyridine, pyrimidine, pyrazine, furan, etc. or benzo-fused analogues of these rings are defined by the term “heteroaryl group.”

The term “substituted heteroaryl group” refers to a heteroaryl group as just described in which one or more hydrogen atoms on any atom of the heteroaryl moiety is replaced by another group such as a halogen, alkyl, substituted alkyl, aryl, substituted aryl, heteroaryl, substituted heteroaryl, alkoxy, aryloxy, boryl, phosphino, amino, silyl, thio, seleno and combinations thereof. Suitable substituted heteroaryl groups include, for example, 4-N,N-dimethylaminopyridine.

The term “alkoxy group” refers to the —OZ′ radical, where Z′ is selected from the group consisting of alkyl, substituted alkyl, cycloalkyl, substituted cycloalkyl, heterocycloalkyl, substituted heterocycloalkyl, silyl groups and combinations thereof as described herein. Suitable alkoxy groups include, for example, methoxy, ethoxy, benzyloxy, t-butoxy, and the like. A related term is “aryloxy” where Z′ is selected from the group consisting of aryl, substituted aryl, heteroaryl, substituted heteroaryl, and combinations thereof. Examples of suitable aryloxy groups include phenoxy, substituted phenoxy, 2-pyridinoxy, 8-quinalinoxy and the like.

The term “alkoxyalkyl group” denotes an alkyl group where at least one of the hydrogen atoms of the alkyl group has been replaced by an alkoxy group. Exemplary alkoxyalkyl groups include methoxymethyl, ethoxymethyl, methoxyethyl, ethoxyethyl, methoxypropyl, ethoxypropyl and isopropoxymethyl. Particular alkoxyalkyl groups include methoxymethyl, methoxyethyl and ethoxymethyl.

The term “alkoxyalkoxy group” denotes an alkoxy group wherein at least one of the hydrogen atoms of the alkoxy group has been replaced by another alkoxy group. Examples of alkoxyalkoxy groups include methoxymethoxy, ethoxymethoxy, methoxyethoxy, ethoxyethoxy, methoxypropoxy and ethoxypropoxy. Particular alkoxyalkoxy groups include methoxymethoxy and methoxyethoxy.

The term “alkoxyalkoxyalkyl group” denotes an alkyl group wherein at least one of the hydrogen atoms of the alkyl group has been replaced by an alkoxyalkoxy group. Examples of alkoxyalkoxyalkyl groups include methoxymethoxymethyl, ethoxymethoxymethyl, methoxyethoxymethyl, ethoxyethoxymethyl, methoxypropoxymethyl, ethoxypropoxymethyl, methoxymethoxyethyl, ethoxymethoxyethyl, methoxyethoxyethyl, ethoxyethoxyethyl, methoxypropoxyethyl and ethoxypropoxyethyl.

The term “amino group” refers to the group —NZ′Z″, where each of Z′ and Z″ is each independently selected from hydrogen; alkyl, substituted alkyl, cycloalkyl, substituted cycloalkyl, heterocycloalkyl, substituted heterocycloalkyl, aryl, substituted aryl, heteroaryl, substituted heteroaryl, alkoxy, alkyloxyalkyl, aryloxy, and combinations thereof.

The term “halogen” means fluoro, chloro, bromo, or iodo, preferably fluoro or chloro.

The term “carbonyl” denotes a —C═O or —C(O)— group.

The term “hydroxy” or “alcohol” denotes a —OH group.

The term “cyano” denotes a —C≡N group

The term “azide” denotes a —N3 group.

The compounds and polymers of the present invention may have asymmetric centers. Compounds and polymers of the present invention containing an asymmetrically substituted atom may be isolated in optically active or racemic forms. According to the Cahn-Ingold-Prelog Convention the asymmetric carbon atom can be of the “R” or “S” configuration. It is well known in the art how to prepare optically active forms, such as by resolution of materials. All chiral, diastereomeric, meso, racemic forms are within the scope of this invention, unless the specific stereochemistry or isomeric form is specifically indicated.

Additionally, as used herein the term C1-C6 alkyl and terms derived therefrom includes all the possible isomeric forms of said C1-C6 alkyl group. Furthermore, the heteroaryl include all the positional isomers. Furthermore, all polymorphic forms and hydrates of monomer (VI), copolymers (I), (IV), (VII), or polymer (VIII) are within the scope of this invention.

The terms “compound” and “a compound of the invention” and “compound of the present invention” and the like, and their plural forms include formula (III) and (VI) and the other copolymers (I), (IV), (VII), or polymer (VIII) described herein and exemplified compounds described herein or a pharmaceutically acceptable salt of each of these formulas. All references to compounds, include all isotopes of the atoms contained therein, including isotopically-labeled compounds.

The terms “polymer” and “a polymer of the invention” and “polymer of the present invention” and the like, and their plural forms include formula (VIII) and monomer (VI), copolymers (I), (IV) and (VII) described herein and exemplified compounds and polymers described herein or a pharmaceutically acceptable salt of each of these formulas. All references to polymers, include all isotopes of the atoms contained therein, including isotopically-labeled polymers.

The compounds and polymers of the present invention may exist as tautomers. All tautomeric forms of the compounds of the invention are contemplated to be within the scope of the present invention.

The compositions also include the prodrugs of monomer (VI), copolymers (I), (IV), (VII), or polymer (VIII). The term prodrug is intended to represent covalently bonded carriers, which are capable of releasing the active ingredient of monomer (VI), copolymers (I), (IV), (VII), or polymer (VIII) respectively, when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups in vivo or by routine manipulation. Prodrugs of monomer (VI), copolymers (I), (IV), (VII), or polymer (VIII) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups), amides (e.g., trifluoroacetylamino, acetylamino, and the like), and the like.

A “pharmaceutically acceptable salt” of a compound means a salt that is pharmaceutically acceptable and that possesses the desired pharmacological activity of the parent compound. Non-limiting examples of such salts include acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are nontoxic. Additional information on suitable pharmaceutically acceptable salts can be found in Remington's Pharmaceutical Sciences, 17th ed., Mack Publishing Company, Easton, Pa., 1985, which is incorporated herein by reference.

An “antigen” includes any substance that may be specifically bound by an antibody molecule. Thus, the term “antigen” encompasses biologic molecules including, but not limited to, simple intermediary metabolites, sugars, lipids, amino acids, and hormones, as well as macromolecules such as complex carbohydrates, phospholipids, nucleic acids, peptides, and proteins, for example ovalbumin (OVA). The antigen may be one that is related to the infection or disease to be treated. The antigen may be from an infectious agent or from a tumor or cancer cell. The antigen may be all or part of a molecule from an infectious agent or tumor/cancer cell. The antigen may be one in which an immune response is desired or intended.

The term “polymer” refers to a molecule composed of repeating structural units connected by covalent chemical bonds often characterized by a substantial number of repeating units (e.g., equal to or greater than 10 repeating units and often equal to or greater than 50 repeating units and often equal to or greater than 100 repeating units) and a high molecular weight (e.g., greater than or equal to 50,000 Da). Polymers are commonly the polymerization product of one or more monomer precursors. The term polymer includes homopolymers, or polymers consisting essentially of a single repeating monomer subunit. The term polymer also includes copolymers which are formed when two or more different types of monomers are linked in the same polymer. Copolymers may comprise two or more monomer subunits, and include random, block, alternating, segmented, grafted, tapered and other copolymers. Useful polymers include organic polymers that are water miscible for vaccine administration.

An “oligomer” refers to a molecule composed of repeating structural units connected by covalent chemical bonds often characterized by a number of repeating units less than that of a polymer (e.g., equal to or less than 10 repeating units) and a lower molecular weights (e.g., less than or equal to about 50,000 Da) than polymers. Oligomers may be the polymerization product of one or more monomer precursors.

It is specifically contemplated that any of m, o, p, p′ or the number of monomers are integers and may be, be at least, or be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 20000, 30000, 40000, 50000, 60000, 70000, 80000, 90000 or more, or any range derivable therein.

The term “operatively linked” refers to a situation where two components are combined to form the active complex prior to binding at the target site. For example, a molecule conjugated to one-half of a biotin-streptavidin complex and an antigen complexed to the other one-half of the biotin-streptavidin complex are operatively linked through complexation of the biotin and streptavidin molecules. The term operatively linked is also intended to refer to covalent or chemical linkages that conjugate two molecules together.

### II. LINKER

Examples of linkers include or exclude chemical moieties and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST). Examples of linkers further comprise a linear carbon chain, such as CN (where N=1-100 carbon atoms). The linker can be a dipeptide linker, such as a valine-citrulline (val-cit), a phenylalanine-lysine (phe-lys) linker, or maleimidocapronic-valine-citruline-p-aminobenzyloxycarbonyl (vc) linker. The linker may be sulfosuccinimidyl-4-[N-maleimidomethyl]cyclohexane-1-carboxylate (smcc). Sulfo-smcc conjugation occurs via a maleimide group which reacts with sulfhydryls (thiols, —SH), while its sulfo-NHS ester is reactive toward primary amines (as found in lysine and the protein or peptide N-terminus). Further, the linker may include or exclude maleimidocaproyl (mc). The covalent linkage may be achieved through the use of Traut's reagent.

The albumin polypeptide, TLR agonist, copolymer, and/or PDS moiety may be covalently linked. For example, the PDS moiety may be covalently linked directly to the TLR agonist (with or without a linker) or to a copolymer comprising the TLR agonist. The linker may include or exclude a bifunctional linker. Examples of amino acids typically found in flexible protein regions may include Gly, Asn and Ser. For example, a suitable peptide linker may be or comprise GGGS (SEQ ID NO:5), GGGSGGGS (SEQ ID NO:6) or (GGGS) n (SEQ ID NO: 6), wherein n=1, 2, 3, 4, 5, 6, 7, 8, 9, or 10 (or any range derivable therein). Other near neutral amino acids, such as Thr and Ala, may also be used in the linker sequence. The length of the linker sequence may vary without significantly affecting the function or activity of the fusion protein (see, e.g., U.S. Pat. No. 6,087,329). The linker may be at least, at most, or exactly 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 amino acid residues (or any range derivable therein).

### III. ALBUMIN

The albumin polypeptide may be from mouse. The albumin polypeptide may be from humans.

The albumin polypeptide may comprise a polypeptide or fragment with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide having the following sequence:

(SEQ ID NO: 1)
MKWVTFISLLFLFSSAYSRGVFRRDAHKSEVAHRFKDLGEENFKALVLIA
FAQYLQQCPFEDHVKLVNEVTEFAKTCVADESAENCDKSLHTLFGDKLCT
VATLRETYGEMADCCAKQEPERNECFLQHKDDNPNLPRLVRPEVDVMCTA
FHDNEETFLKKYLYEIARRHPYFYAPELLFFAKRYKAAFTECCQAADKAA
CLLPKLDELRDEGKASSAKQRLKCASLQKFGERAFKAWAVARLSQRFPKA
EFAEVSKLVTDLTKVHTECCHGDLLECADDRADLAKYICENQDSISSKLK
ECCEKPLLEKSHCIAEVENDEMPADLPSLAADFVESKDVCKNYAEAKDVF
LGMFLYEYARRHPDYSVVLLLRLAKTYKTTLEKCCAAADPHECYAKVFDE
FKPLVEEPQNLIKQNCELFEQLGEYKFQNALLVRYTKKVPQVSTPTLVEV
SRNLGKVGSKCCKHPEAKRMPCAEDYLSVVLNQLCVLHEKTPVSDRVTKC
CTESLVNRRPCFSALEVDETYVPKEFNAETFTFHADICTLSEKERQIKKQ
TALVELVKHKPKATKEQLKAVMDDFAAFVEKCCKADDKETCFAEEGKKLV
AASRAALGL.

The albumin polypeptide may comprise a polypeptide or fragment with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide having the following sequence:

(SEQ ID NO: 2)
EAHKSEIAHRYNDLGEQHFKGLVLIAFSQYLQKCSYDEHAKLVQEVTDFA
KTCVADESAANCDKSLHTLFGDKLCAIPNLRENYGELADCCTKQEPERNE
CFLQHKDDNPSLPPFERPEAEAMCTSFKENPTTFMGHYLHEVARRHPYFY
APELLYYAEQYNEILTQCCAEADKESCLTPKLDGVKEKALVSSVRQRMKC
SSMQKFGERAFKAWAVARLSQTFPNADFAEITKLATDLTKVNKECCHGDL
LECADDRAELAKYMCENQATISSKLQTCCDKPLLKKAHCLSEVEHDTMPA
DLPAIAADFVEDQEVCKNYAEAKDVFLGTFLYEYSRRHPDYSVSLLLRLA
KKYEATLEKCCAEANPPACYGTVLAEFQPLVEEPKNLVKTNCDLYEKLGE
YGFQNAILVRYTQKAPQVSTPTLVEAARNLGRVGTKCCTLPEDQRLPCVE
DYLSAILNRVCLLHEKTPVSEHVTKCCSGSLVERRPCFSALTVDETYVPK
EFKAETFTFHSDICTLPEKEKQIKKQTALAELVKHKPKATAEQLKTVMDD
FAQFLDTCCKAADKDTCFSTEGPNLVTRCKDALA.

The albumin polypeptide may comprise a polypeptide or fragment with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide having the following sequence:

(SEQ ID NO: 3)
MKWVTFLLLLFVSGSAFSRGVFRREAHKSEIAHRYNDLGEQHFKGLVLIA
FSQYLQKCSYDEHAKLVQEVTDFAKTCVADESAANCDKSLHTLFGDKLCA
IPNLRENYGELADCCTKQEPERNECFLQHKDDNPSLPPFERPEAEAMCTS
FKENPTTFMGHYLHEVARRHPYFYAPELLYYAEQYNEILTQCCAEADKES
CLTPKLDGVKEKALVSSVRQRMKCSSMQKFGERAFKAWAVARLSQTFPNA
DFAEITKLATDLTKVNKECCHGDLLECADDRAELAKYMCENQATISSKLQ
TCCDKPLLKKAHCLSEVEHDTMPADLPAIAADFVEDQEVCKNYAEAKDVF
LGTFLYEYSRRHPDYSVSLLLRLAKKYEATLEKCCAEANPPACYGTVLAE
FQPLVEEPKNLVKTNCDLYEKLGEYGFQNAILVRYTQKAPQVSTPTLVEA
ARNLGRVGTKCCTLPEDQRLPCVEDYLSAILNRVCLLHEKTPVSEHVTKC
CSGSLVERRPCFSALTVDETYVPKEFKAETFTFHSDICTLPEKEKQIKKQ
TALAELVKHKPKATAEQLKTVMDDFAQFLDTCCKAADKDTCFSTEGPNLV
TRCKDALA.

The albumin polypeptide may comprise a polypeptide or fragment with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide having the following sequence:

(SEQ ID NO: 4)
DAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNE
CFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKC
ASLQKFGERAFKAWAVARLSQRFPKAEFAEVSKLVTDLTKVHTECCHGDL
LECADDRADLAKYICENQDSISSKLKECCEKPLLEKSHCIAEVENDEMPA
DLPSLAADFVESKDVCKNYAEAKDVFLGMFLYEYARRHPDYSVVLLLRLA
KTYKTTLEKCCAAADPHECYAKVFDEFKPLVEEPQNLIKQNCELFEQLGE
YKFQNALLVRYTKKVPQVSTPTLVEVSRNLGKVGSKCCKHPEAKRMPCAE
DYLSVVLNQLCVLHEKTPVSDRVTKCCTESLVNRRPCFSALEVDETYVPK
EFNAETFTFHADICTLSEKERQIKKQTALVELVKHKPKATKEQLKAVMDD
FAAFVEKCCKADDKETCFAEEGKKLVAASRAALG.

### IV. TLR AGONISTS

### A. Copolymers Comprising TLR Agonist

Described herein is a copolymer having the structure (I):

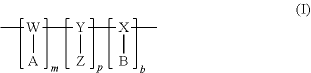

where A is absent or includes at least one group that binds an Antigen Presenting Cell (APC) mannose receptor or includes mannose-binding C-type lectin; Z includes at least one Toll-Like Receptor (TLR) agonist; B includes a pyridyl disulfide moiety or a maleimide moiety; W, Y, and X are each independently a monomer unit of a polymer; m is from 0 to 100000, from 5 to 50000, from 5 to 10000, from 5 to 1000, from 0 to 150; p is from 0 to 100000, from 5 to 50000, from 5 to 10000, from 5 to 1000, from 0 to 15; b is from 1 to 100000, from 5 to 50000, from 5 to 10000, from 5 to 1000, from 1 to 10; m may be from 0 to 150, p is from 1 to 10, and b is from 1 to 10. It is understood that m, p, and b are integers. m may be 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150, or any range derivable therein. p may be 0, 1, 2, 3, 4, 5, 6, 7, 8, 9, or 10, or any derivable range therein. b may be 1, 2, 3, 4, 5, 6, 7, 8, 9, or 10, or any derivable range therein.

Also described is a compound comprising:

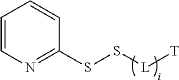

wherein L is a linker, T is a TLR agonist, and i is 0 or 1.

Also described is a compound comprising:

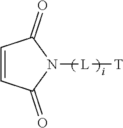

wherein L is a linker, T is a TLR agonist, and i is 0 or 1. Also provided are compositions comprising the polymers and compounds of the disclosure. Methods include a method for treating cancer in a subject comprising administering a polymer, compound, or composition of the disclosure to the subject. Also described is a method for targeting a TLR agonist to a tumor in a subject comprising administering a polymer, compound, or composition of the disclosure to the subject. Methods provide for a method for increasing the accumulation of a TLR agonist in a tumor in a subject, the method comprising administering a polymer, compound, or composition of the disclosure to the subject.

W, Y, and X may be each independently polymerized monomer units of polyacrylate, a polyacrylamide, a saturated polyolefin, a polyamide, a peptide, a polypeptide, an unsaturated olefin formed by ring opening metathesis polymerization, a siloxane, a polysiloxane, a polyether, a polysaccharide, a polyoxazoline, a polyimine, a polyvinyl derivative or any combination thereof. W, Y, and X may be independently selected from acrylic acid, methacrylic acid, acrylamide, methacrylamide, N-(2-hydroxypropyl) methacrylamide, N-(2-hydroxyethyl) methacrylamide, natural and un-natural amino acids, silanols, monosaccharides, glycols, substituted and unsubstituted 2-oxazolines N-substituted and unsubstituted aziridine, substituted and unsubstituted 2-ethyl-2-oxazoline.

The TLR agonist may have the general structure (II),

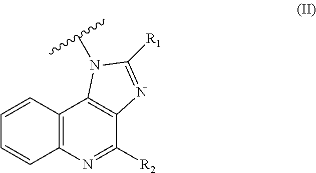

wherein R₁ and R₂ are each independently a hydrogen atom, a halogen, an alkyl group, a substituted alkyl group, a heteroalkyl group, a substituted heteroalkyl group, a cycloalkyl group, a substituted cycloalkyl group, a heterocycloalkyl group, a substituted heterocycloalkyl group, an aryl group, a substituted aryl group, a heteroaryl group, a substituted heteroaryl group, an alkoxy group, an alkoxyalkyl group, an alkoxyalkoxy group, and alkoxyalkoxyalkyl group, an amino group, or a hydroxyl group.

Moiety

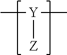

may be further defined as

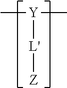

wherein L′ is a substituted or unsubstituted alkyl group comprising from 1 to 9 carbon atoms, a substituted or unsubstituted ralkyl group comprising from 1 to 9 carbon atoms, or an ethylene glycol moiety comprising from 1 to 9 ethylene glycol groups. L′ may comprise 1, 2, 3, 4, 5, 6, 7, 8, or 9 carbon atoms, or any derivable range therein.

Z may be

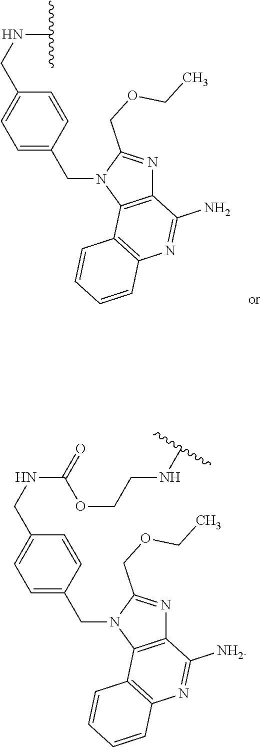 [SMILES: *NCc1ccc(Cn2c(COCC)nc3c(N)nc4ccccc4c32)cc1 ; *NCCOC(=O)NCc1ccc(Cn2c(COCC)nc3c(N)nc4ccccc4c32)cc1]

Moiety:

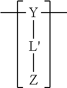

may be

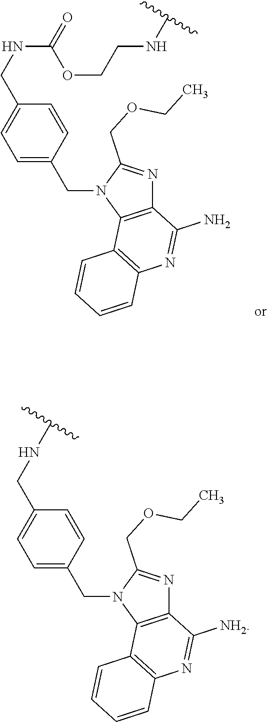 [SMILES: *NCCOC(=O)NCc1ccc(Cn2c(COCC)nc3c(N)nc4ccccc4c32)cc1 ; *NCc1ccc(Cn2c(COCC)nc3c(N)nc4ccccc4c32)cc1]

Moiety:

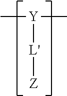

may be

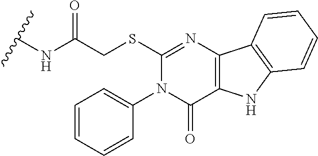 [SMILES: *NC(=O)CSc1nc2c([nH]c3ccccc32)c(=O)n1-c1ccccc1]

Moiety:

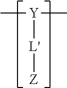

may be

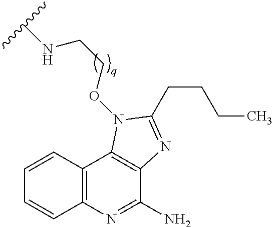

wherein q is 2 to 9. q may be 2, 3, 4, 5, 6, 7, 8, or 9, or any derivable range therein.

Moiety:

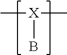

may be further defined as

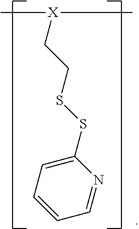

Moiety:

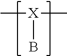

may be further defined as

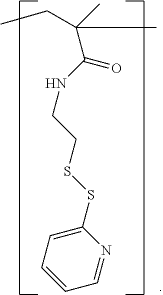

Moiety:

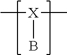

may be further defined as

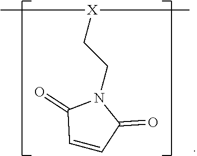

Moiety:

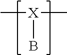

may be further defined as

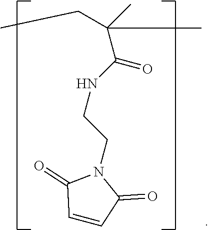

Moiety:

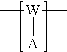

may be further defined as

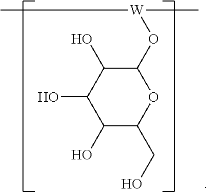

Moiety:

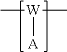

may be further defined as

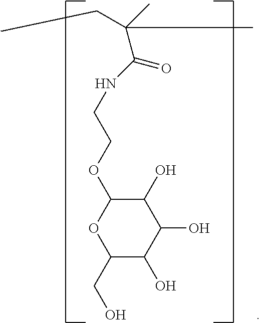

Moiety:

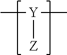

may be further defined as

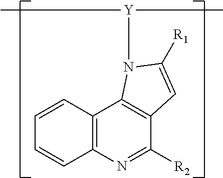

wherein R₁ and R₂ are each independently a hydrogen atom, a halogen, an alkyl group, a substituted alkyl group, a heteroalkyl group, a substituted heteroalkyl group, a cycloalkyl group, a substituted cycloalkyl group, a heterocycloalkyl group, a substituted heterocycloalkyl group, an aryl group, a substituted aryl group, a heteroaryl group, a substituted heteroaryl group, an alkoxy group, an alkoxyalkyl group, an alkoxyalkoxy group, and alkoxyalkoxyalkyl group, an amino group, or a hydroxyl group.

Moiety:

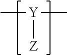

may be further defined as

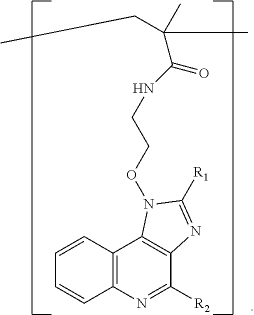

The polymer may comprise at least one TLR agonist and at least one group that binds to an Antigen Presenting Cell (APC) mannose receptor.

The polymer may be further defined as

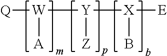

wherein E and Q are end units and each independently comprises a residue of the polymer, a fluorescent molecule, an albumin polypeptide, a dithioate group, an azide, a linker, an immunomodulating agent, a pyridyl disulfide moiety, a maleimide moiety, a TLR agonist, a group that binds to an Antigen Presenting Cell (APC) mannose receptor, or combinations thereof.

The dithioate group may be further defined as

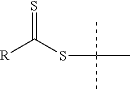

wherein R is an alkyl or aryl group containing from 1 to 12 carbon atoms. R may have 1, 2, 3, 4, 5, 6, 7, 8, 9, 10, 11, or 12 carbon atoms, or any derivable range therein.

The linker may comprise a polyethylene glycol moiety or a hydrocarbon moiety. The polyethylene glycol linker may comprise from 1 to 150 polyethylene glycol units. The polyethylene glycol linker may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 polyethylene glycol units, or any derivable range therein. The hydrocarbon moiety may comprise from 1 to 150 methylene units. The hydrocarbon moiety may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 methylene units, or any derivable range therein. The polymer may be a copolymer, wherein W, X, and/or Y are different. W, X, and/or Y may be the same. m may be at least 1, p=0, and at least one of Q or E comprises a TLR agonist. The TLR agonist may comprise a CpG TLR agonist. The CpG TLR agonist may comprise ODN1826. The CpG TLR agonist may comprise a phoshorothioated backbone. W, Y, and X can be provided in any order.

The compound may be further defined as

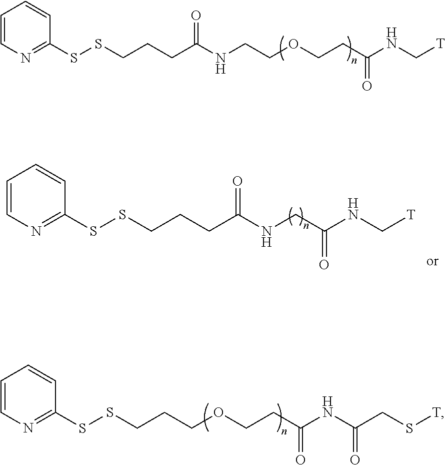

wherein n is from 1 to 150.

The compound may be further defined as

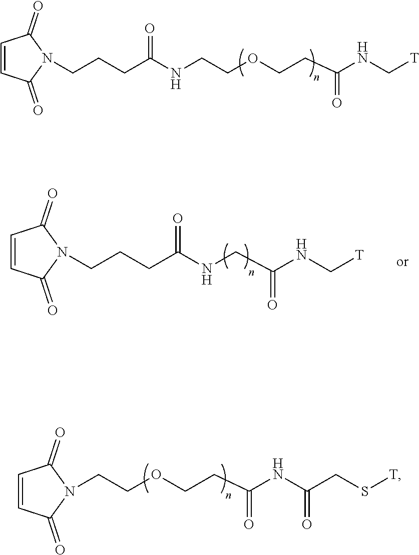

wherein n is from 1 to 150.

The TLR agonist may have the general structure (II),

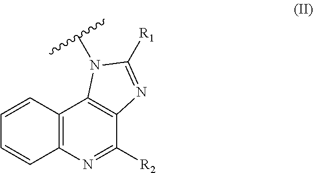

wherein R₁ and R₂ are each independently a hydrogen atom, a halogen, an alkyl group, a substituted alkyl group, a heteroalkyl group, a substituted heteroalkyl group, a cycloalkyl group, a substituted cycloalkyl group, a heterocycloalkyl group, a substituted heterocycloalkyl group, an aryl group, a substituted aryl group, a heteroaryl group, a substituted heteroaryl group, an alkoxy group, an alkoxyalkyl group, an alkoxyalkoxy group, and alkoxyalkoxyalkyl group, an amino group, or a hydroxyl group.

T may be

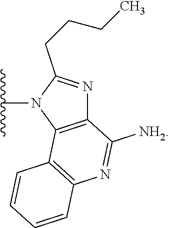 [SMILES: *n1c(CCCC)nc2c(N)nc3ccccc3c21]

T may be

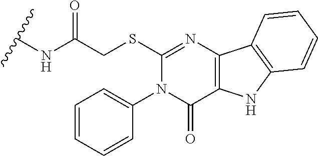 [SMILES: *NC(=O)CSc1nc2c([nH]c3ccccc32)c(=O)n1-c1ccccc1]

In one application, linkers include compounds for molecular conjugation reactions to provide structural stability or assistance in protein-protein, protein-peptide, protein-polymer, polymer-small molecule, peptide/protein-small molecule interactions, immobilization for assays or purification, as well as various peptide-nucleic acid and nucleic acid-nucleic acid conjugations, among many others. Typically, linkers contain functional groups, such as primary amines, sulfhydryls, acids, alcohols, azides, alkynes and halides. Specifically, maleimide (sulfhydryl reactive) and succinimidyl ester (NHS) or isothiocyanate (ITC) groups that react with amines may find use in the current aspects.

A bifunctional linker can be used as a latent spacer between a therapeutic or diagnostic moiety and a polymer. The latency may be selected such that a first linking group (functional group) of the bifunctional linker can be selectively conjugated in the presence of a second linking group. The latency can be selected such that after both linking groups on the bifunctional linker are conjugated one group can be selectively cleaved. For example, the hydrolysis of the spacer-polymer bond can be rate limiting in the release of the therapeutic or diagnostic moiety from the polymeric prodrug. Cleavage and release of the therapeutic or diagnostic moiety from the polymeric prodrug can occur in vivo, for example by an enzymatic or non-enzymatic hydrolysis mechanism using linking groups such as ester, carbonate, carbamate, imine (hydrazone), amide, maleimide, succinimidyl, vinylsulfone, conjugated C═C double bond, epoxy, aldehyde, ketone, silane or siloxane functionalities. It is within the purview of those skilled in the art to appreciate the release of a therapeutic or diagnostic moiety from polymeric prodrugs employing aqueous hydrolysis depends on a multitude of factors like hydration of the linkage, the nature of the leaving group and steric crowding around the linkage. Substrate specificity, hydrophilicity, and steric crowding all influence the release from enzyme susceptible linkages and subtle changes made to the specific aspects disclosed herein can still obtain the same result without departed from the spirit and scope of the invention. The bifunctional linkers can be first conjugated with the copolymers and polymers of the current invention through a first functional group on the bifunctional linker and then the product can be further conjugated through a second functional group on the bifunctional linker. Once both functional groups of the bifunctional linker are conjugated, the portion derived from the bifunctional linker can be referred to as a linking group or linker. Exemplary compounds used as bifunctional linkers in the preparation of the polymeric conjugated vaccines of the current invention including any of the above mentioned functional groups can include alkyne-PEG5-acid, N-alloc-1,4-butandiamine hydrochloride, N-alloc-1,6-hexanediamine hydrochloride, allyl(4-methoxyphenyl)dimethylsilane, 6-(allyloxycarbonylamino)-1-hexanol, 3-(allyloxycarbonylamino)-1-propanol, 4-aminobutyraldehyde diethyl acetal, (E)-N-(2-aminoethyl)-4-{2-[4-(3-azidopropoxy)phenyl]diazenyl}benzamide hydrochloride, N-(2-aminoethyl)maleimide trifluoroacetate salt, amino-PEG4-alkyne, benzyl N-(3-hydroxypropyl) carbamate, 4-(Boc-amino)-1-butanol, 4-(Boc-amino)butyl bromide, 2-(Boc-amino) ethanethiol, 2-[2-(Boc-amino) ethoxy]ethoxyacetic acid, (dicyclohexylammonium) salt, 2-(Boc-amino)ethyl bromide, 6-(Boc-amino)-1-hexanol, 21-(Boc-amino)-4,7,10,13,16,19-hexaoxaheneicosanoic acid, 6-(Boc-amino) hexyl bromide, 5-(Boc-amino)-1-pentanol, 3-(Boc-amino)-1-propanol, 3-(Boc-amino) propyl bromide, 15-(Boc-amino)-4,7,10,13-tetraoxapentadecanoic acid, N-Boc-1,4-butanediamine, N-Boc-cadaverine, N-Boc-ethanolamine, N-Boc-ethylenediamine, N-Boc-2,2′-(ethylenedioxy) diethylamine, N-Boc-1,6-hexanediamine, N-Boc-1,6-hexanediamine hydrochloride, N-Boc-4-isothiocyanatoaniline, N-Boc-4-isothiocyanatobutylamine, N-Boc-2-isothiocyanatocthylamine, N-Boc-3-isothiocyanatopropylamine, N-Boc-N-methylethylenediamine, N-Boc-m-phenylenediamine, N-Boc-p-phenylenediamine, 2-(4-Boc-1-piperazinyl)acetic acid, N-Boc-1,3-propanediamine, N-Boc-1,3-propanediamine, N-Boc-N′-succinyl-4,7,10-trioxa-1,13-tridecanediamine, N-Boc-4,7,10-trioxa-1,13-tridecanediamine, N-(4-Bromobutyl) phthalimide, 4-bromobutyric acid, 4-bromobutyryl chloride, 4-bromobutyryl chloride, N-(2-bromoethyl) phthalimide, 6-bromo-1-hexanol, 3-(bromomethyl)benzoic acid N-succinimidylester, 4-(bromomethyl)phenyl isothiocyanate, 8-bromooctanoic acid, 8-bromo-1-octanol, 4-(2-bromopropionyl) phenoxyacetic acid, N-(3-bromopropyl) phthalimide, 4-(tert-Butoxymethyl)benzoic acid, tert-butyl 2-(4-{[4-(3-azidopropoxy)phenyl]azo}benzamido)ethylcarbamate, 2-[2-(tert-butyldimethylsilyloxy)ethoxy]ethanamine, tert-butyl 4-hydroxybutyrate, chloral hydrate, 4-(2-chloropropionyl)phenylacetic acid, 1,11-diamino-3,6,9-trioxaundecane, di-Boc-cystamine, diethylene glycol monoallyl ether, 3,4-Dihydro-2H-pyran-2-methanol, 4-[(2,4-Dimethoxyphenyl) (Fmoc-amino)methyl]phenoxyacetic acid, 4-(Diphenylhydroxymethyl)benzoic acid, 4-(Fmoc-amino)-1-butanol, 2-(Fmoc-amino) ethanol, 2-[2-(Fmoc-amino)ethoxy]ethylamine hydrochloride, 2-(Fmoc-amino)ethyl bromide, 6-(Fmoc-amino)-1-hexanol, 5-(Fmoc-amino)-1-pentanol, 3-(Fmoc-amino)-1-propanol, 3-(Fmoc-amino) propyl bromide, N-Fmoc-2-bromoethylamine, N-Fmoc-1,4-butanediamine hydrobromide, N-Fmoc-cadaverine hydrobromide, N-Fmoc-ethylenediamine hydrobromide, N-Fmoc-1,6-hexanediamine hydrobromide, N-Fmoc-1,3-propanediamine hydrobromide, N-Fmoc-N”-succinyl-4,7,10-trioxa-1,13-tridecanediamine, (3-Formyl-1-indolyl)acetic acid 6-Guanidinohexanoic acid 4-Hydroxybenzyl alcohol N-(4-hydroxybutyl)trifluoroacetamide, 4′-hydroxy-2,4-dimethoxybenzophenone, N-(2-hydroxyethyl) maleimide, 4-[4-(1-hydroxyethyl)-2-methoxy-5-nitrophenoxy]butyric acid, N-(2-hydroxyethyl)trifluoroacetamide, N-(6-hydroxyhexyl)trifluoroacetamide, 4-hydroxy-2-methoxybenzaldehyde, 4-hydroxy-3-methoxybenzyl alcohol, 4-(hydroxymethyl)benzoic acid, 4-hydroxymethyl-3-methoxyphenoxyacetic acid, 4-(4-hydroxymethyl-3-methoxyphenoxy)butyric acid, 4-(hydroxymethyl)phenoxyacetic acid, 3-(4-hydroxymethylphenoxy)propionic acid, N-(5-hydroxypentyl)trifluoroacetamide, 4-(4′-hydroxyphenylazo)benzoic acid, N-(3-hydroxypropyl)trifluoroacetamide, 2-maleimidoethyl mesylate, 4-mercapto-1-butanol, 6-mercapto-1-hexanol, phenacyl 4-(bromomethyl)phenylacetate, phenacyl 4-(bromomethyl)phenylacetate, 4-sulfamoylbenzoic acid, 4-sulfamoylbutyric acid, N-trityl-1,2-ethanediamine hydrobromide, 4-(Z-amino)-1-butanol, 6-(Z-amino)-1-hexanol, 5-(Z-amino)-1-pentanol, N—Z-1,4-butanediamine hydrochloride, N—Z-ethanolamine, N—Z-ethylenediamine hydrochloride, N—Z-ethylenediamine hydrochloride, N—Z-1,6-hexanediamine hydrochloride, N—Z-1,5-pentanediamine hydrochloride, and N—Z-1,3-propanediamine hydrochloride. Non-limiting examples of trifunctional linkers used to link three separate molecules together include N1,N4-bis-Boc-spermidine, N1,N5-bis-Boc-spermidine, N-Boc-diethanolamine, N1-Boc-2,2′-iminodiethylamine, N-Boc-iminodipropionic acid, N1-Boc-3,3′-iminodipropylamine, N,N″-Di-Z-diethylenetriamine. The bifunctional linker may contain a radical conjugation functional group, such as found in 2-(2-(2-(2-azidoethoxy) ethoxy) ethoxy)ethyl 4-cyano-4-(phenylcarbonothioylthio)pentanoate that can be first conjugated with monomers in a polymerization reaction (i.e., reversible addition-fragmentation chain transfer (RAFT) polymerization) to afford an azide functionalized agent. The azide agent can then be used in subsequent conjugation reactions to prepare polymer conjugate vaccines or polymer conjugate vaccine precursors. A non-limiting example of a commercial source of the above mentioned bifunctional and trifunctional linkers is Sigma Aldrich® (U.S.A).

### B. Compounds of Formula (II)

The TLR agonist may comprise a compound of formula II as described below. Low toxicity, small molecule Toll-Like Receptor (TLR)-7 and/or TLR-8 imidazoquinoline ligands may be provided as vaccine adjuvants with decreased hydrophobicity (c Log P) and increased activity for use in vaccine formulations. The TLR7 agonist, TLR8 agonist, or TLR7/8 agonist monomer may have the general structure (VI):

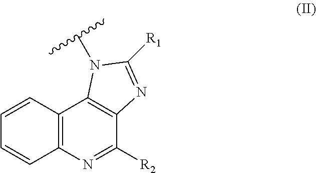

where R₁ and R₂ are each independently a hydrogen atom, a halogen, an alkyl group, a substituted alkyl group, a heteroalkyl group, a substituted heteroalkyl group, a cycloalkyl group, a substituted cycloalkyl group, a heterocycloalkyl group, a substituted heterocycloalkyl group, an aryl group, or a substituted aryl group; and R₃ is a ligand comprising a polymerizable group Y′.

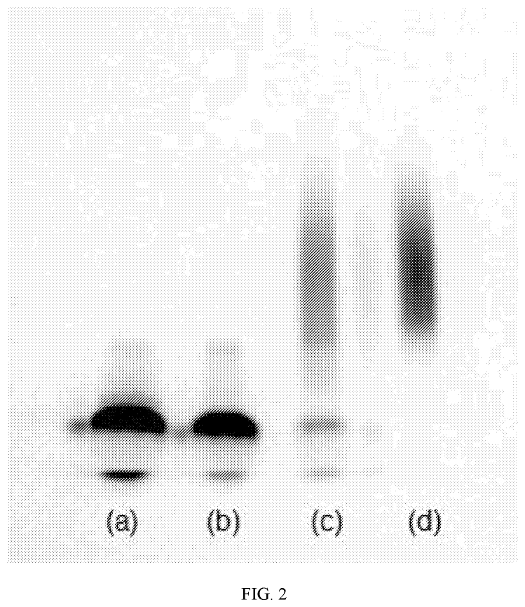

Non-limiting examples of imidazoquinoline compounds of general structure (VI) for use in the current compounds and compositions of the disclosure that are easily derived using various commercially available acid chlorides (e.g., substituting for (14) in the synthetic protocol described herein in FIG. 2, step vi) include or exclude 2-methacrylamidoethyl 4-((4-amino-2-methyl-1H-imidazo[4,5-c]quinolin-1-yl)methyl)benzylcarbamate, 2-methacrylamidoethyl 4-((4-amino-2-ethyl-1H-imidazo[4,5-c]quinolin-1-yl)methyl)benzylcarbamate, 2-methacrylamidoethyl 4-((4-amino-2-propyl-1H-imidazo[4,5-c]quinolin-1-yl)methyl)benzylcarbamate, 2-methacrylamidoethyl 4-((4-amino-2-isopropyl-1H-imidazo[4,5-c]quinolin-1-yl)methyl)benzylcarbamate, 2-methacrylamidoethyl 4-((4-amino-2-cyclopropyl-1H-imidazo[4,5-c]quinolin-1-yl)methyl)benzylcarbamate, 2-methacrylamidoethyl 4-((4-amino-2-butyl-1H-imidazo[4,5-c]quinolin-1-yl)methyl)benzylcarbamate, 2-methacrylamidoethyl 4-((4-amino-2-isobutyl-1H-imidazo[4,5-c]quinolin-1-yl)methyl)benzylcarbamate, 2-methacrylamidoethyl 4-((4-amino-2-sec-butyl-1H-imidazo[4,5-c]quinolin-1-yl)methyl)benzylcarbamate, 2-methacrylamidoethyl 4-((4-amino-2-cyclobutyl-1H-imidazo[4,5-c]quinolin-1-yl)methyl)benzylcarbamate, 2-methacrylamidoethyl 4-((4-amino-2-(oxetan-2-yl)-1H-imidazo[4,5-c]quinolin-1-yl)methyl)benzylcarbamate, 2-methacrylamidoethyl 4-((4-amino-2-(oxetan-3-yl)-1H-imidazo[4,5-c]quinolin-1-yl)methyl)benzylcarbamate, 2-methacrylamidoethyl 4-((4-amino-2-cyclopentyl-1H-imidazo[4,5-c]quinolin-1-yl)methyl)benzylcarbamate, 2-methacrylamidoethyl 4-((4-amino-2-(tetrahydrofuran-3-yl)-1H-imidazo[4,5-c]quinolin-1-yl)methyl)benzylcarbamate, 2-methacrylamidoethyl 4-((4-amino-2-(tetrahydrofuran-2-yl)-1H-imidazo[4,5-c]quinolin-1-yl)methyl)benzylcarbamate, 2-methacrylamidoethyl 4-((4-amino-2-(2-methoxyethyl)-1H-imidazo[4,5-c]quinolin-1-yl)methyl)benzylcarbamate, 2-methacrylamidoethyl 4-((4-amino-2-(ethoxymethyl)-1H-imidazo[4,5-c]quinolin-1-yl)methyl)benzylcarbamate (TLR-7-oxyethyl-methacrylamide (TLR-MA, 3)), 2-methacrylamidoethyl 4-((4-amino-2-((2-methoxyethoxy)methyl)-1H-imidazo[4,5-c]quinolin-1-yl)methyl)benzylcarbamate, 4-amino-1-(4-(((2-methacrylamidoethoxy)carbonylamino)methyl)benzyl)-1H-imidazo[4,5-c]quinoline-2-carboxylic acid, methyl 4-amino-1-(4-(((2-methacrylamidoethoxy)carbonylamino)methyl)benzyl)-1H-imidazo[4,5-c]quinoline-2-carboxylate, ethyl 4-amino-1-(4-(((2-methacrylamidoethoxy)carbonylamino)methyl)benzyl)-1H-imidazo[4,5-c]quinoline-2-carboxylate, 2-(4-amino-1-(4-(((2-methacrylamidoethoxy)carbonylamino)methyl)benzyl)-1H-imidazo[4,5-c]quinolin-2-yl)acetic acid, methyl 2-(4-amino-1-(4-(((2-methacrylamidoethoxy)carbonylamino)methyl)benzyl)-1H-imidazo[4,5-c]quinolin-2-yl)acetate, ethyl 2-(4-amino-1-(4-(((2-methacrylamidoethoxy)carbonylamino)methyl)benzyl)-3-(4-amino-1-(4-(((2-1H-imidazo[4,5-c]quinolin-2-yl)acetate, methacrylamidoethoxy)carbonylamino)methyl)benzyl)-1H-imidazo[4,5-c]quinolin-2-yl)propanoic acid, methyl 3-(4-amino-1-(4-(((2-methacrylamidoethoxy)carbonylamino)methyl)benzyl)-1H-imidazo[4,5-c]quinolin-2-yl)propanoate, ethyl 3-(4-amino-1-(4-(((2-methacrylamidoethoxy)carbonylamino)methyl)benzyl)-1H-imidazo[4,5-c]quinolin-2-yl)propanoate, 2-methacrylamidoethyl 4-((4-amino-2-(thiazol-2-yl)-1H-imidazo[4,5-c]quinolin-1-yl)methyl)benzylcarbamate, 2-methacrylamidoethyl 4-((4-amino-2-(thiazol-5-yl)-1H-imidazo[4,5-c]quinolin-1-yl)methyl)benzylcarbamate, 2-methacrylamidoethyl 4-((4-amino-2-(isothiazol-5-yl)-1H-imidazo[4,5-c]quinolin-1-yl)methyl)benzylcarbamate, 2-methacrylamidoethyl 4-((4-amino-2-(isothiazol-3-yl)-1H-imidazo[4,5-c]quinolin-1-2-methacrylamidoethyl 4-((4-amino-2-(isothiazol-4-yl)-1H-yl)methyl)benzylcarbamate, imidazo[4,5-c]quinolin-1-yl)methyl)benzylcarbamate, 2-methacrylamidoethyl 4-((4-amino-2-(thiazol-4-yl)-1H-imidazo[4,5-c]quinolin-1-yl)methyl)benzylcarbamate, 2-methacrylamidoethyl 4-((4-amino-2-(oxazol-4-yl)-1H-imidazo[4,5-c]quinolin-1-yl)methyl)benzylcarbamate, 2-methacrylamidoethyl 4-((4-amino-2-(isoxazol-4-yl)-1H-imidazo[4,5-c]quinolin-1-yl)methyl)benzylcarbamate, 2-methacrylamidoethyl 4-((4-amino-2-(isoxazol-3-yl)-1H-imidazo[4,5-c]quinolin-1-yl)methyl)benzylcarbamate, 2-methacrylamidoethyl 4-((4-amino-2-(furan-2-yl)-1H-imidazo[4,5-c]quinolin-1-yl)methyl)benzylcarbamate, 2-methacrylamidoethyl 4-((4-amino-2-(furan-3-yl)-1H-imidazo[4,5-c]quinolin-1-yl)methyl)benzylcarbamate, 2-methacrylamidoethyl 4-((4-amino-2-(thiophen-3-yl)-1H-imidazo[4,5-c]quinolin-1-yl)methyl)benzylcarbamate, 2-methacrylamidoethyl 4-((4-amino-2-(thiophen-2-yl)-1H-imidazo[4,5-c]quinolin-1-yl)methyl)benzylcarbamate, 2-methacrylamidoethyl 4-((4-amino-2-(oxazol-2-yl)-1H-imidazo[4,5-c]quinolin-1-yl)methyl)benzylcarbamate, 2-methacrylamidoethyl 4-((4-amino-2-(oxazol-5-yl)-1H-imidazo[4,5-c]quinolin-1-yl)methyl)benzylcarbamate, 2-4-((4-amino-2-(isoxazol-5-yl)-1H-imidazo[4,5-c]quinolin-1-methacrylamidoethyl yl)methyl)benzylcarbamate, 2-methacrylamidoethyl 4-((4-amino-2-(pyridin-2-yl)-1H-imidazo[4,5-c]quinolin-1-yl)methyl)benzylcarbamate, 2-methacrylamidoethyl 4-((4-amino-2-(pyridin-3-yl)-1H-imidazo[4,5-c]quinolin-1-yl)methyl)benzylcarbamate, 2-methacrylamidoethyl 4-((4-amino-2-(pyridin-4-yl)-1H-imidazo[4,5-c]quinolin-1-yl)methyl)benzylcarbamate, 2-methacrylamidoethyl 4-((4-amino-2-(pyrimidin-4-yl)-1H-imidazo[4,5-c]quinolin-1-yl)methyl)benzylcarbamate, 2-methacrylamidoethyl 4-((4-amino-2-(pyrazin-2-yl)-1H-imidazo[4,5-c]quinolin-1-yl)methyl)benzylcarbamate, 2-methacrylamidoethyl 4-((4-amino-2-(pyridazin-3-yl)-1H-imidazo[4,5-c]quinolin-1-yl)methyl)benzylcarbamate, 2-methacrylamidoethyl 4-((4-amino-2-(pyridazin-4-yl)-1H-imidazo[4,5-c]quinolin-1-yl)methyl)benzylcarbamate, 2-methacrylamidoethyl 4-((4-amino-2-(pyrimidin-5-yl)-1H-imidazo[4,5-c]quinolin-1-yl)methyl)benzylcarbamate, and derivatives thereof. Also disclosed are compounds of formula (1) where R3=OR5. Exemplary imidazoquinoline derivatives further include: N-(3-(4-amino-2-(ethoxymethyl)-1H-imidazo[4,5-c]quinolin-1-yloxy) propyl) methacrylamide, N-(4-(4-amino-2-(ethoxymethyl)-1H-imidazo[4,5-c]quinolin-1-yloxy)butyl) methacrylamide, N-(4-(4-amino-2-(ethoxymethyl)-1H-imidazo[4,5-c]quinolin-1-yloxy) pentyl) methacrylamide, N-(4-(4-amino-2-(ethoxymethyl)-1H-imidazo[4,5-c]quinolin-1-yloxy) hexyl) methacrylamide, N-(4-(4-amino-2-(ethoxymethyl)-1H-imidazo[4,5-c]quinolin-1-yloxy) heptyl) methacrylamide, N-(4-(4-amino-2-(ethoxymethyl)-1H-imidazo[4,5-c]quinolin-1-yloxy) octyl) methacrylamide, N-(4-(4-amino-2-(ethoxymethyl)-1H-imidazo[4,5-c]quinolin-1-yloxy) nonyl) methacrylamide, N-(4-(4-amino-2-(ethoxymethyl)-1H-imidazo[4,5-c]quinolin-1-yloxy) decyl) methacrylamide, as well as those N—O bond containing structures containing the above mentioned 4- and 2-position imidazoquinoline substitutions and derivatives thereof. The 4-amino group in common to the above mentioned variously 2-substituted imidazoquinoline derivatives installed in FIG. 2 (step vii) could be any other nucleophile capable of a nucleophilic aromatic substitution (SNAr) reaction with 4-substituted heteroaryl chloride, for example hydroxide, methylamine, dimethylamine, ethylamine, methylethylamine, propylamine, azetidine, cyclopropylamine, pyrrolidine, etc. Alternatively the 4-substituted heteroaryl chloride may be replaced with hydrogen by a hydro or radical dehalogenation reaction or participate as a coupling partner in a transition metal catalyzed carbon-carbon bond formation reaction.

Any of the disclosed TLR agonist monomers can be linked with another monomer that contains at least one group that binds to an Antigen Presenting Cell (APC) mannose receptor as polymers, homopolymers, copolymers, copolymeric blends, terpolymers, quaterpolymers, or oligomers, etc., and can be present in compositions and conjugated to, for example, antigens. Any of the copolymers may be a block copolymer, an alternating copolymer or a random copolymer. Preferably the compounds, copolymers, and polymers of the present invention are hydrophilic. Non-limiting examples of water-soluble polymers include or exclude polyacrylates, such as poly(acrylic acid) or poly(methacrylic acid) or poly(hydroxypropyl methacrylate), polyamides, such as poly(acrylamide) or poly(methacrylamide), polysaccharides, polyoxazoline, such as poly(ethyloxazoline), polyimine, such as poly(ethylenimine), and polyvinyl derivatives, such as a poly(vinylalcohol) or poly(vinylpyrrolidone). Linking of monomers to form polymers, homopolymers, copolymers, polymeric blends, terpolymers, quaterpolymers, or oligomers and conjugation to, for instance, antigens as disclosed herein, may be accomplished using synthetic organic techniques using polymerizable and linking groups which would be readily apparent to one of ordinary skill in the art, based on the present disclosure. Non-limiting examples of making the compounds, copolymers, and polymers of the present invention are provided in the Examples section.

### C. Other TLR Agonists

The copolymer of formula I may be operably linked to a TLR agonist. The TLR agonist may be a compound of general formula (VI), as described herein. The TLR agonist may be one known in the art and/or described herein. The TLR agonists may include an agonist to TLR1 (e.g., peptidoglycan or triacyl lipoproteins), TLR2 (e.g., lipoteichoic acid; peptidoglycan from Bacillus subtilis, E. coli 0111: B4, Escherichia coli K12, or Staphylococcus aureus; atypical lipopolysaccharide (LPS) such as Leptospirosis LPS and Porphyromonas gingivalis LPS; a synthetic diacylated lipoprotein such as FSL-1 or Pam2CSK4; lipoarabinomannan or lipomannan from M. smegmatis; triacylated lipoproteins such as Pam3CSK4; lipoproteins such as MALP-2 and MALP-404 from mycoplasma; Borrelia burgdorferi OspA; Porin from Neisseria meningitidis or Haemophilus influenza; Propionibacterium acnes antigen mixtures; Yersinia LcrV; lipomannan from Mycobacterium or Mycobacterium tuberculosis; Trypanosoma cruzi GPI anchor; Schistosoma mansoni lysophosphatidylserine; Leishmania major lipophosphoglycan (LPG); Plasmodium falciparum glycophosphatidylinositol (GPI); zymosan; antigen mixtures from Aspergillus fumigatus or Candida albicans; and measles hemagglutinin), TLR3 (e.g., double-stranded RNA, polyadenylic-polyuridylic acid (Poly(A:U)); polyinosine-polycytidylic acid (Poly(I:C)); polyinosine-polycytidylic acid high molecular weight (Poly(I:C) HMW); and polyinosine-polycytidylic acid low molecular weight (Poly(I:C) LMW)), TLR4 (e.g., LPS from Escherichia coli and Salmonella species); TLR5 (e.g., Flagellin from B. subtilis, P. aeruginosa, or S. typhimurium), TLR8 (e.g., single stranded RNAs such as ssRNA with 6UUAU repeats, RNA homopolymer (ssPolyU naked), HIV-1 LTR-derived ssRNA (ssRNA40), or ssRNA with 2 GUCCUUCAA repeats (ssRNA-DR)), TLR7 (e.g., imidazoquinoline compound imiquimod, Imiquimod VacciGrade™, Gardiquimod VacciGrade™, or Gardiquimod™; adenine analog CL264; base analog CL307; guanosine analog loxoribine; TLR7/8 (e.g., thiazoquinoline compound CL075; imidazoquinoline compound CLO97, R848, or R848 VacciGrade™), TLR9 (e.g., CpG ODNs); and TLR11 (e.g., Toxoplasma gondii Profilin). The TLR agonist may be a specific agonist listed above. The TLR agonist may be one that agonizes either one TLR or two TLRs specifically.

The TLR agonist may be a TLR7, TLR8, or a TLR7/8 agonist. The TLR agonist may be multiple (polymerized) molecules of the same TLR agonist or may be a mixture of linked different TLR agonists. The TLR agonist may be linked or polymerized by methods known in the art and/or described herein. The compound (e.g., TLR agonist) may be water soluble. Water solubility affects the shelf-life, stability, and pharmaceutical composition of the compound. Due to the structure of TLR7 and TLR8, most TLR7 and/or TLR8 agonists are poorly soluble in water. However, the compounds of Formula (I) have the advantage of water solubility.

### V. ADDITIONAL THERAPIES

### A. Immunotherapy

The methods may include or exclude administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies useful in the methods of the disclosure are described below.

### 1. Checkpoint Inhibitors and Combination Treatment

Methods may include or exclude administration of immune checkpoint inhibitors (also referred to as checkpoint inhibitor therapy), which are further described below.

b. PD-1, PDL1, and PDL2 inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. PD-1, PDL1, and PDL2 may be human PD-1, PDL1 and PDL2.

The PD-1 inhibitor may be a molecule that inhibits the binding of PD-1 to its ligand binding partners. The PD-1 ligand binding partners may be PDL1 and/or PDL2. A PDL1 inhibitor may be a molecule that inhibits the binding of PDL1 to its binding partners. PDL1 binding partners may be PD-1 and/or B7-1. The PDL2 inhibitor may be a molecule that inhibits the binding of PDL2 to its binding partners. A PDL2 binding partner may be PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

The PD-1 inhibitor may be an anti-PD-1 antibody (e.g., a human antibody, a humanized antibody, or a chimeric antibody). The anti-PD-1 antibody may be selected from the group consisting of nivolumab, pembrolizumab, and pidilizumab. The PD-1 inhibitor may be an immunoadhesin (e.g., an immunoadhesin comprising an extracellular or PD-1 binding portion of PDL1 or PDL2 fused to a constant region (e.g., an Fc region of an immunoglobulin sequence). The PDL1 inhibitor may comprise AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

The immune checkpoint inhibitor may be a PDL1 inhibitor such as Durvalumab, also known as MEDI4736, atezolizumab, also known as MPDL3280A, avelumab, also known as MSB00010118C, MDX-1105, BMS-936559, or combinations thereof. The immune checkpoint inhibitor may be a PDL2 inhibitor such as rHIgM12B7.

The inhibitor may comprise the heavy and light chain CDRs or VRs of nivolumab, pembrolizumab, or pidilizumab. Accordingly, the inhibitor may comprise the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. The antibody may be one that competes for binding with and/or binds to the same epitope on PD-1, PDL1, or PDL2 as the above-mentioned antibodies. The antibody may have at least about 70, 75, 80, 85, 90, 95, 97, or 99% (or any derivable range therein) variable region amino acid sequence identity with the above-mentioned antibodies.

c. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. The inhibitor may be one that blocks the CTLA-4 and B7-1 interaction. The inhibitor may be one that blocks the CTLA-4 and B7-2 interaction.

The immune checkpoint inhibitor may be an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO01/14424).

The inhibitor may comprise the heavy and light chain CDRs or VRs of tremelimumab or ipilimumab. Accordingly, the inhibitor may comprise the CDR1, CDR2, and CDR3 domains of the VH region of tremelimumab or ipilimumab, and the CDR1, CDR2 and CDR3 domains of the VL region of tremelimumab or ipilimumab. The antibody may be one that competes for binding with and/or binds to the same epitope on PD-1, B7-1, or B7-2 as the above-mentioned antibodies. The antibody may have at least about 70, 75, 80, 85, 90, 95, 97, or 99% (or any derivable range therein) variable region amino acid sequence identity with the above-mentioned antibodies.

### 2. CD40 Agonists

The immunotherapy may include or exclude CD40 agonists, such as anti-CD40 agonist antibodies. Agonistic CD40 antibodies are known in the art and include, for example, APX005M, ChiLob7/4, ADC-1013, SEA-CD40, selicrelumab, and CDX-1140.

### 3. Dendritic Cell Therapy

The immunotherapy may include or exclude dendritic cell therapy. Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor.

### 4. CAR-T Cell Therapy

The immunotherapy may include or exclude CAR-T cell therapy. Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some aspects, the CAR-T therapy targets CD19.

### 5. Cytokine Therapy

The immunotherapy may include or exclude cytokine therapy. Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in antiviral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy. Also contemplated within the scope of the disclosure is the administration of chemokine therapy as an immunotherapy useful in the methods of the disclosure.

### 6. Adoptive T-Cell Therapy

The immunotherapy may include or exclude adoptive T-cell therapy. Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death. [60]

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Patients include or exclude those that have been previously treated for a therapy described herein, are currently being treated for a therapy described herein, or have not been treated for a therapy described herein. The patient may be one that has been determined to be resistant to a therapy described herein. The patient may be one that has been determined to be sensitive to a therapy described herein.

### B. Oncolytic Virus

The additional therapy may include or exclude oncolytic virus. The additional therapy may comprise an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy

### C. Polysaccharides

The additional therapy may include or exclude polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### D. Neoantigens

The additional therapy may include or exclude neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

### E. Chemotherapies

The additional therapy may include or exclude a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-a), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane).

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. Appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. The chemotherapeutic agent may be administered in an amount effective to cause arrest or regression of the cancer in a host, when the chemotherapy is administered with the construct. The chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### F. Radiotherapy

The additional therapy or prior therapy may comprise radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

The amount of ionizing radiation may be greater than 20 Gy and is administered in one dose. The amount of ionizing radiation may be 18 Gy and is administered in three doses. The amount of ionizing radiation may be at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). The ionizing radiation may be administered in at least, at most, or exactly 1, 2, 3, 4, 5, 6, 7, 8, 9, or 10 doses (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

The amount of IR may be presented as a total dose of IR, which is then administered in fractionated doses. For example, the total dose may be 50 Gy administered in 10 fractionated doses of 5 Gy each. The total dose may be 50-90 Gy, administered in 20-60 fractionated doses of 2-3 Gy each. The total dose of IR may be at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). The total dose may be administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. At least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses may be administered (or any derivable range therein). At least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses may be administered per week.

### G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### H. Other Agents

It is contemplated that other agents may be used in combination with certain aspects of the present aspects to improve the therapeutic efficacy of treatment. These additional agents include or exclude agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other aspects, cytostatic or differentiation agents can be used in combination with certain aspects of the present aspects to improve the anti-hyperproliferative efficacy of the treatments. Inhibitors of cell adhesion are contemplated to improve the efficacy of the present aspects. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present aspects to improve the treatment efficacy.

### VI. PROTEINACEOUS COMPOSITIONS

The polypeptides of the disclosure such as those comprising or encoding for albumin may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID Nos: 1-4.

The polypeptides or polynucleotides of the disclosure may include 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein, of SEQ ID NO: 1-4.

In some aspects, the polypeptide comprises amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) of SEQ ID NOs: 1-4.

In some aspects, the polypeptide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) contiguous amino acids of SEQ ID NOs: 1-4.

In some aspects, the polypeptide comprises at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) contiguous amino acids of any of SEQ ID NOs: 1-4 and starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 of any of SEQ ID NO:1-4.

In some aspects, the polypeptide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) contiguous amino acids of SEQ ID NOs: 1-4 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of SEQ ID NOS: 1-4.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions.

The substitution may be at amino acid position or nucleic acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 of one of SEQ ID NO:1-4.

The polypeptides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more amino acids (or any derivable range therein).

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria, eukaryotic cells, yeast, or mammalian cells. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In other aspects, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated by the inventors that various changes may be made in the DNA sequences of genes with appreciable alteration of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific aspects, all or part of proteins described herein can also be synthesized in solution or on a solid support in accordance with conventional techniques. Various automatic synthesizers are commercially available and can be used in accordance with known protocols. Sec, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One aspect includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

### VII. THERAPEUTIC METHODS

The current methods and compositions relate to methods for treating cancer. In some aspects, the cancer comprises a solid tumor. In some aspects, the cancer is non-lymphatic. In some aspects, the cancer is melanoma, lymphoma, bladder, breast, or colon cancer.

The compositions of the disclosure may be used for in vivo, in vitro, or ex vivo administration. The route of administration of the composition may be, for example, intratumoral, intracutaneous, subcutaneous, intravenous, intralymphatic, and intraperitoneal administrations. In some aspects, the administration is intratumoral or intralymphatic or peri-tumoral. In some aspects, the compositions are administered directly into a cancer tissue or a lymph node.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder.” and “tumor” are not mutually exclusive as referred to herein.

The cancers amenable for treatment include, but are not limited to, tumors of all types, locations, sizes, and characteristics. The methods and compositions of the disclosure are suitable for treating, for example, pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, specific breast cancers such as ductal carcinoma in situ, invasive ductal carcinoma, tubular carcinoma of the breast, medullary carcinoma of the breast, mucinous carcinoma of the breast, papillary carcinoma of the breast, cribriform carcinoma of the breast, invasive lobular carcinoma, inflammatory breast cancer, lobular carcinoma in situ, male breast cancer, paget's disease of the nipple, phyllodes tumors of the breast, recurrent and/or metastatic breast, cancer, luminal A or B breast cancer, triple-negative/basal-like breast cancer, and HER2-enriched breast cancer, lymphoid cancer, bronchial adenomas/carcinoids, tracheal cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pincoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

### VIII. PHARMACEUTICAL COMPOSITIONS AND METHODS

In some aspects, pharmaceutical compositions are administered to a subject. Different aspects involve administering an effective amount of a composition to a subject. In some aspects, a composition comprising an inhibitor may be administered to the subject or patient to treat cancer or reduce the size of a tumor. Additionally, such compounds can be administered in combination with an additional cancer therapy.

Compositions can be formulated for parenteral administration, e.g., formulated for injection via the intravenous, transcatheter injection, intraarterial injection, intramuscular, subcutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and, the preparations can also be emulsified. The preparation of such formulations will be known to those of skill in the art in light of the present disclosure. Other routes of administration include intratumoral, peri-tumoral, intralymphatic, injection into cancer tissue, and injection into lymph nodes. In some aspects, the administration is systemic.

Other routes of administration are also contemplated. For example, the constructs and agents may be administered in association with a carrier. In some aspects, the carrier is a nanoparticle or microparticle. In some aspects, the nanoparticle or microparticle is a tumor directed nanoparticle or microparticle. For example, the carrier may further comprise a targeting moiety that directs the carrier to the tumor. The targeting moiety may be a binding agent (e.g. antibody, including scFv, etc. or other antigen binding agent) that specifically recognizes tumor cells. In some aspects, the construct is enclosed within the carrier. In some aspects, the construct is covalently or non-covalently attached to the surface of the carrier. In some aspects, the carrier is a liposome. In further aspects, a carrier molecule described herein is excluded.

Particles can have a structure of variable dimension and known variously as a microsphere, microparticle, nanoparticle, nanosphere, or liposome. Such particulate formulations can be formed by covalent or non-covalent coupling of the construct to the particle. In some aspects, particles described herein are excluded.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. Pharmaceutically acceptable salts include the conventional non-toxic salts or the quaternary ammonium salts of the parent compound formed, for example, from non-toxic inorganic or organic acids. The pharmaceutically acceptable salts can be synthesized from the parent compound which contains a basic or acidic moiety by conventional chemical methods.

Some variation in dosage will necessarily occur depending on the condition of the subject. The person responsible for administration will, in any event, determine the appropriate dose for the individual subject. An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the effects desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

Typically, for a human adult (weighing approximately 70 kilograms), from about 0.1 mg to about 3000 mg (including all values and ranges there between), or from about 5 mg to about 1000 mg (including all values and ranges there between), or from about 10 mg to about 100 mg (including all values and ranges there between), of a compound are administered. It is understood that these dosage ranges are by way of example only, and that administration can be adjusted depending on the factors known to the skilled artisan.

In certain aspects, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or μg/kg or micrograms/kg/minute or mg/kg/min or micrograms/kg/hour or mg/kg/hour, or any range derivable therein.

A dose may be administered on an as needed basis or every 1, 2, 3, 4, 5, 6, 7, 8, 9, 10, 11, 12, 18, or 24 hours, or 1, 2, 3, 4, 5, 6, 7, 8, 9, 10, 11, 12, 13, 14, 15, 16, 17, 18, 19, or 20 days (or any range derivable therein) or 1, 2, 3, 4, 5, 6, 7, 8, 9, or times per day (or any range derivable therein). A dose may be first administered before or after signs of a condition. In some aspects, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of the condition (or any range derivable therein). The patient may be treated for 1, 2, 3, 4, 5, 6, 7, 8, 9, 10 or more days (or any range derivable therein) or until symptoms of the condition have disappeared or been reduced or after 6, 12, 18, or 24 hours or 1, 2, 3, 4, or 5 days after tumor or indications of cancer.

### IX. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Cancer Vaccines with Direct Adjuvant Binding

### A. Methods and Results

### 1. Mice and Cancer Cell Lines

Female C57BL/6 mice (aged 8-12 weeks) were purchased from Charles River Laboratory. Female Balb/c mice (aged 8-12 weeks) were purchased from Jackson Laboratory. B16F10 melanoma, CT26 and MC38 colon carcinoma, and EMT6 mammary carcinoma cell lines were purchased from ATCC and cultured according to instructions. Tumor inoculations were 500,000 cells in 30 μL sterile PBS injected subcutaneously into the shaved back of mice unless otherwise noted. Tumor dimensions were measured with digital calipers and volume was calculated as length*width*height*π/6. All of the animal experiments performed in this research were approved by the Institutional Animal Care and Use Committee of the University of Chicago. Cell lines were routinely checked for mycoplasma contamination.

### 2. Polymer Synthesis

N-(2-(pyridin-2-yldisulfancyl)ethyl) methacrylamide (PDS monomer), mannose monomer, TLR7 monomer (murine), and TLR7/8 (human) monomer were synthesized as previously described (37). 2-hydroxypropyl methacrylamide was purchased from Sigma Aldrich and recrystallized in acetone prior to use. TLR4 monomer was synthesized as in Scheme 1 (38). The azide-terminated trithocarbonate chain transfer agent (CTA) was synthesized as previously described. Monomers and CTA were solubilized in 1 mL anhydrous dimethyl sulfoxide (DMSO) in desired molar ratios, keeping the total monomer mass below 300 mg. Feed ratios for relevant polymers are given in Table 1. Photo initiator Eosin Y (Fisher Scientific) was added at a ratio of [1]:[50] relative to CTA. The solution was placed in a 5 mL Schlenk flask and degassed via three freeze pump-thaw cycles. Reaction was started by placing the flask in a glass dish lined with green LED strip lights. The reaction and lighted dish together were covered with foil and allowed to stir over night. After 14 hours, the reaction was stopped by submerging the flask in liquid nitrogen. The polymer was then precipitated from cold diethyl ether three times with solubilization in methanol between washes. The light pink precipitate was dried overnight in a vacuum oven then removed for NMR and GPC analysis.

TABLE 1
Feed ratios for synthesized polymers. Ratios given relative to CTA.

| | | TLR7 | | | |
|---|---|---|---|---|---|
| Polymer | Mannose | (or 7/8) | TLR4 | PDS | HPMA |
| PDS binding experiments | — | — | — | 10 | 50 |
| PDS non-binding control | — | — | — | — | 70 |
| TLR7 efficacy experiments | 20 | 10 | — | 10 | 50 |
| TLR4 efficacy experiments | 20 | — | 10 | 10 | 50 |
| TLR7 non-binding control | 20 | 10 | — | — | 68 |
| TLR4 non-binding control | 20 | — | 10 | — | 68 |
| TLR9 or TLR2/6 | 10 | — | — | 10 | 50 |
| terminated polymers | | | | | |

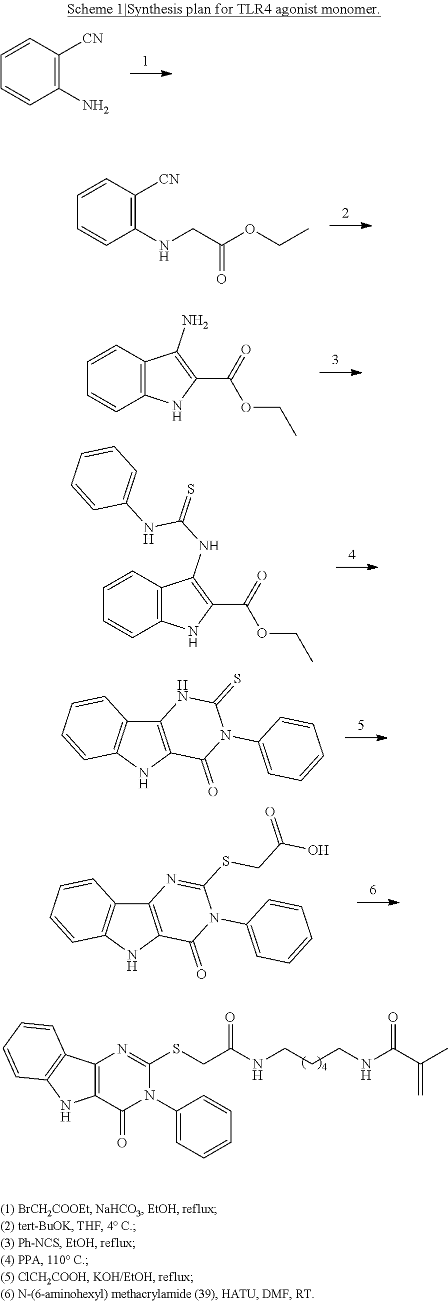

3. Conjugation of CpG to p(Man-PDS-HPMA)

Modified single stranded amine-C6-5′-CpG-ODN1826, TCCATGACGTTCCTGACGTT-3′-6-FAM (amine-CPG-FAM), with a phosphorothioated backbone was purchased from Integrated DNA Technologies and dissolved at 10 mg/mL in PBS. (2,5-dioxopyrrolidin-1-yl) 4-(2-azatricyclo[10.4.0.04,9]hexadeca-1 (16),4,6,8,12,14-hexaen-10-yn-2-yl)-4-oxobutanoate (DBCONHS) was purchased from Sigma Aldrich and dissolved in DMF at 100 mg/mL. 1 mL of amine-CPG-FAM solution (10 mg) was added to 1 mL of DMF, followed by 100 μL of potassium phosphate buffer (pH 8.0). 20 molar equivalents of DBCO-NHS in DMF was then added to amine-CPG-FAM mixture. Solution was then stirred in the dark at room temperature for 4 hours. Reaction was monitored on an Agilent 6130 quadrupole LC-MS system with HPLC analysis conducted on a 1260 Infinity HPLC system using a HYPERSIL GOLD C8 3 UM 100 mm×4.6 mm LC column (Thermo Scientific) using a gradient of mobile phases consisting of 0.1 M Triethylammonium Acetate Buffer in Water (Phase A) to 100% acetonitrile (Phase B). Mass analysis was conducted using negative mode electrospray ionization. Agilent OpenLab CDS software was then used to deconvolute the charge-state ladder into the target mass. Reaction was then diluted to 8 mL using PBS, and excess DBCO-NHS was removed using Zeba desalting columns with a 7K MWCO (Thermo Scientific). Next, 3 molar equivalents of p(Man-PDS-HPMA) in 1 mL PBS was added to the reaction mixture was stirred overnight in the dark at room temperature. Conjugation to polymer was confirmed as an increase in molecular weight via gel electrophoresis (Mini-PROTEAN TGX Stain-Free Gel (Bio-Rad)). CPG-FAM-p(Man-PDS-HPMA) conjugate was then purified on an AKTA-Avant 25 (Cytiva), using a HiLoad 16/600 Superdex 75 pg (Cytiva) size exclusion column using phosphate buffer to yield purified CPG-FAM-p(Man-PDS-HPMA) construct.

### 4. In Vitro Reporter Macrophage Activity of CPG-FAM-p(Man-PDS-HPMA)

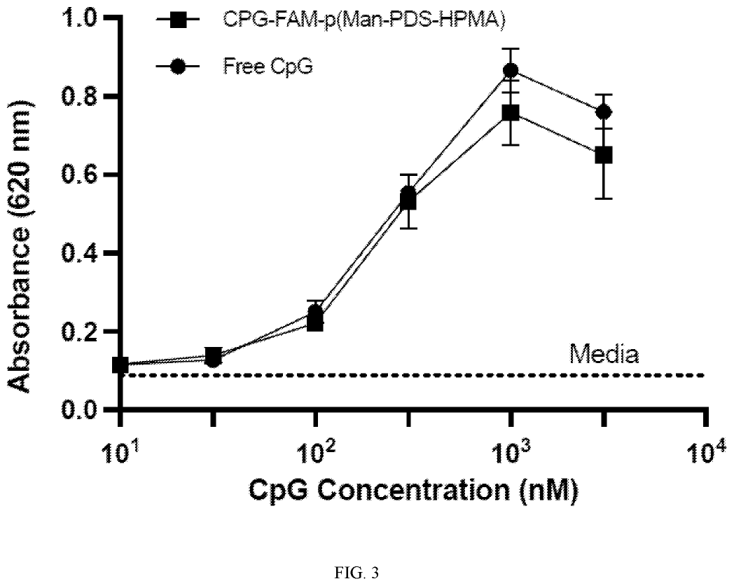

To confirm the immunostimulatory activity of the conjugated CpG, the inventors incubated pure CPG-FAM-p(Man-PDS-HPMA) with RAW-Blue Mouse Macrophage Reporter Cells (InvivoGen). CPG-FAM-p(Man-PDS-HPMA) was serially diluted and incubated with cells according manufacturer's instructions. Concentration of CPG-FAM-p(Man-PDS-HPMA) was measured using a NanoDrop 2000 Spectrophotometer (Thermo Scientific) using an extinction coefficient of 75000 at 495 nm. Serial dilutions of free CpG ODN 1826 (InvivoGen) were tested alongside CPG-FAM-p(Man-PDS-HPMA). After 20 hours of incubation, 20 μL of cell supernatant was incubated with 180 μL of QUANTI-Blue Solution (InvivoGen) for 1 hour to detect NF-κB/AP-1 secretion. Optical density was then evaluated via an Epoch Microplate Spectrophotometer (BioTek Instruments). These results are shown in FIG. 3.

5. In Vitro Tumor Cell Binding of p(PDS-HPMA)

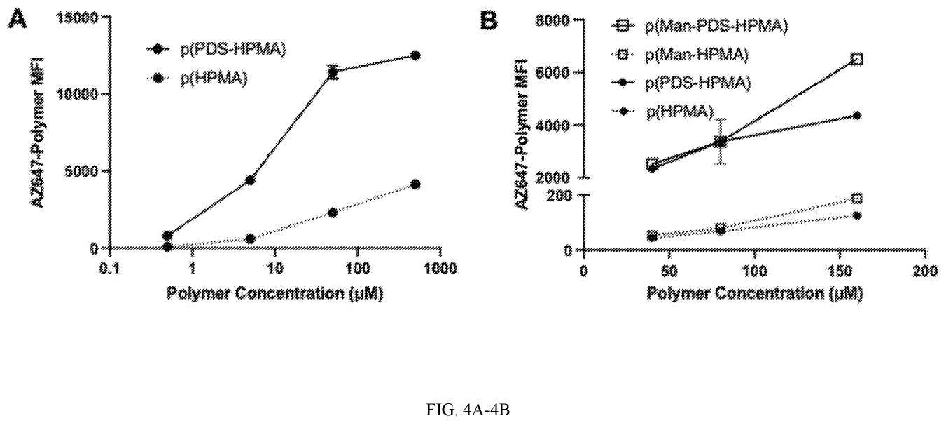

The inventors first evaluated whether the PDS-containing polymers could successfully bind exofacial protein thiols on tumor cells in vitro. For detection, AZDye 647 DBCO (Click Chemistry Tools) was conjugated to polymer via the azide chain end and unconjugated dye was removed using Zeba desalting columns with a 7K MWCO (Thermo Scientific). Cells were removed from culture, washed free of media with PBS two times, then incubated on ice with various concentrations of dye labeled polymer for 90 minutes. After incubation, cells were washed twice with PBS and resuspended in 2% heat inactivated FBS in PBS for flow cytometric analysis. Cells were acquired on BD LSRFortessa and data were analyzed via FlowJo. Mean Fluorescence Intensity (MFI) of singlet cells was recorded and compared between groups (FIG. 4).

Most importantly, binding was observed to be concentration dependent and significantly higher for the PDS containing polymer—p(PDS-HPMA)—than for the “spacer” monomer alone-p(HPMA) (FIG. 4). It was also important to confirm that the binding would not be significantly impacted by the presence of the mannose monomer, since tumor cells have been reported to occasionally express mannose receptors. This importantly confirmed that binding is independent of mannose monomer, meaning future studies can attribute binding to PDS alone and that the platform will still show significant binding for other applications where DC-targeting and receptor-mediated endocytosis is not required. The results of these studies were also confirmed on CT26 and MC38 murine tumor cell lines.

6. In Vivo Tumor Cell Binding of p(PDS-HPMA)

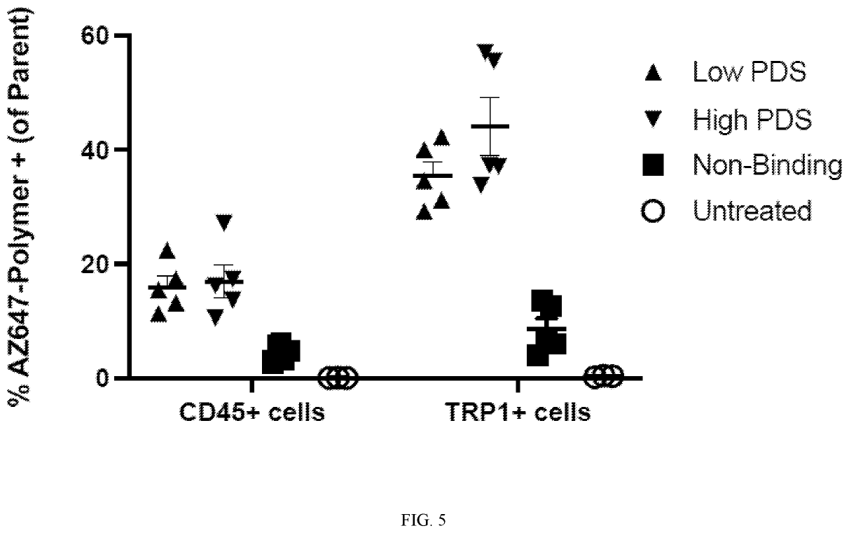

The inventors next evaluated in vivo binding to tumors via flow cytometry. B16F10 tumor were inoculated intradermally as described previously. Once tumors reached an average size of ˜80 mm³, 30 μL dye labeled polymer (molecular weight matched, containing high or low weight fraction PDS, or none for non-binding control) was injected intratumorally at a concentration of 200 μM fluorophore. Three hours after injection, tumors were collected and digested for 30 min at 37° C. Digestion medium was pyruvate free DMEM (Gibco) supplemented with 5% FBS, 3.3 mg/mL collagenase D (Sigma-Aldrich), 1 mg/mL collagenase IV (Worthington Biochemical) and 1.2 mM CaCl₂). Single-cell suspensions were prepared using a 70 μm cell strainer (Thermo Fisher Scientific). Red blood cells were lysed with 3 mL ACK lysing buffer (Gibco) for 90 sec and neutralized with 15 mL DMEM supplemented with 5% FBS. Cell viability was determined using LIVE/DEAD Fixable Violet Dead Cell Stain (405 nm excitation, Invitrogen). Staining with PE anti-mouse CD45 (clone 30-F11, BioLegend) and Alexa Fluor 488 anti-TRP1 (clone EPR21960, Abcam) was done in 2% FBS in PBS. Frequency of AZ647-polymer positive cells was measured and compared between cell populations and injection types (FIG. 5).

The inventors were interested in confirming polymer binding to tumor cells in a murine cancer model. CD45 is the leukocyte common antigen and appears on all cells of hematopoietic origin including basophils, neutrophils, eosinophils, monocytes, macrophages, NK cells, and T and B cells. The inventors saw significant binding with the PDS containing polymer to TRP1+ tumor cells, but not to CD45+ immune cells (FIG. 5). They can therefore reasonably conclude that the polymer binds preferentially to cancer cells over immune cells in the tumor, meaning the platform can be used to target tumor cell surfaces. They can also conclude that increasing the number of PDS groups per chain does not lead to significantly better binding.

7. Toxicity—PDS Binding Decreases Production of Systemic Pro-Inflammatory Cytokines Associated with Intratumoral p(Man-TLR7) Injection

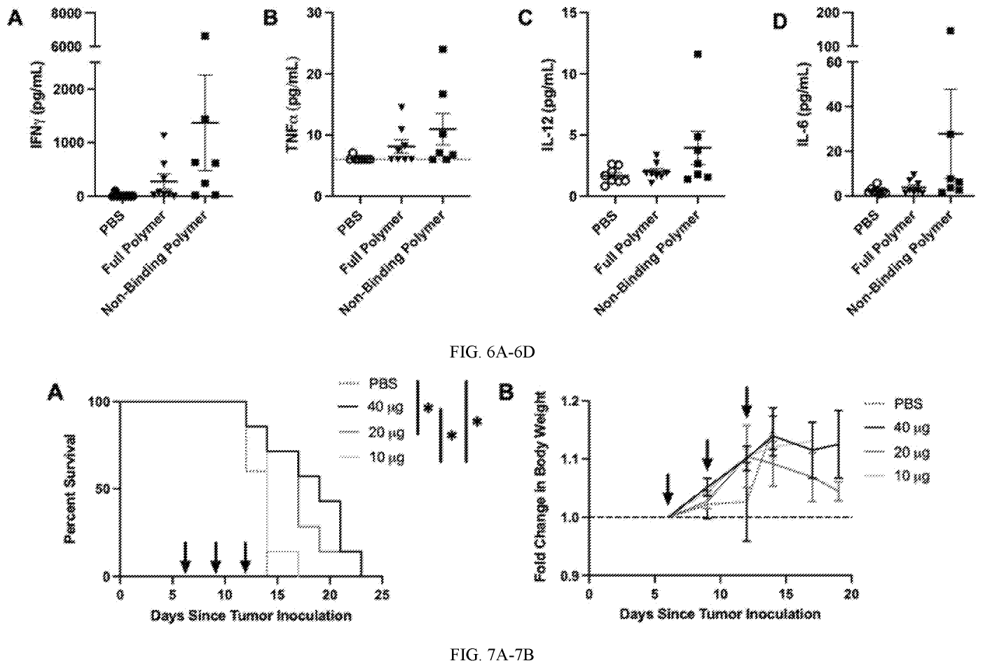

The inventors evaluated the toxicity of the p(Man-TLR7-PDS) platform. Mice bearing established B16F10 tumors were injected intratumorally with 40 μg TLR7 equivalent (as quantified by absorbance at 273 nm, relative to a standard curve) polymer in 30 μL sterile PBS on days 6 and 9. On day 10, plasma was collected via submandibular bleed and analyzed for pro-inflammatory cytokines using LEGENDplex Mouse Inflammation Panel Assay (BioLegend) (FIG. 6).

Here the inventors demonstrate the effectiveness of the PDS-binding polymer in reducing toxicity associated with intratumorally injected immune adjuvant p(Man-TLR7) (FIG. 6). The inventors hypothesize that a similar reduction can be obtained with other agonists when conjugated to or incorporated in multivalent PDS-containing polymers.

8. Antitumor Efficacy—p(Man-TLR7-PDS) is Nontoxic and Significantly Prolongs Survival

The inventors started by evaluating the dose-dependent efficacy and toxicity of the full p(Man-TLR7-PDS-HPMA) polymer. Mice bearing established B16F10 tumors were injected intratumorally with 10, 20, or 40 μg TLR7 equivalent (as quantified by absorbance at 327 nm, relative to a standard curve) polymer in 30 μL sterile PBS on days 6, 9, and 12 after tumor inoculation. Mouse weights were recorded starting on day 6 to observe toxicity until the tumor reached a volume of 1000 mm³ and the mice were euthanized (FIG. 7).

9. Antitumor Efficacy—PDS Binding Improves Therapeutic Efficacy of p(Man-TLR7)

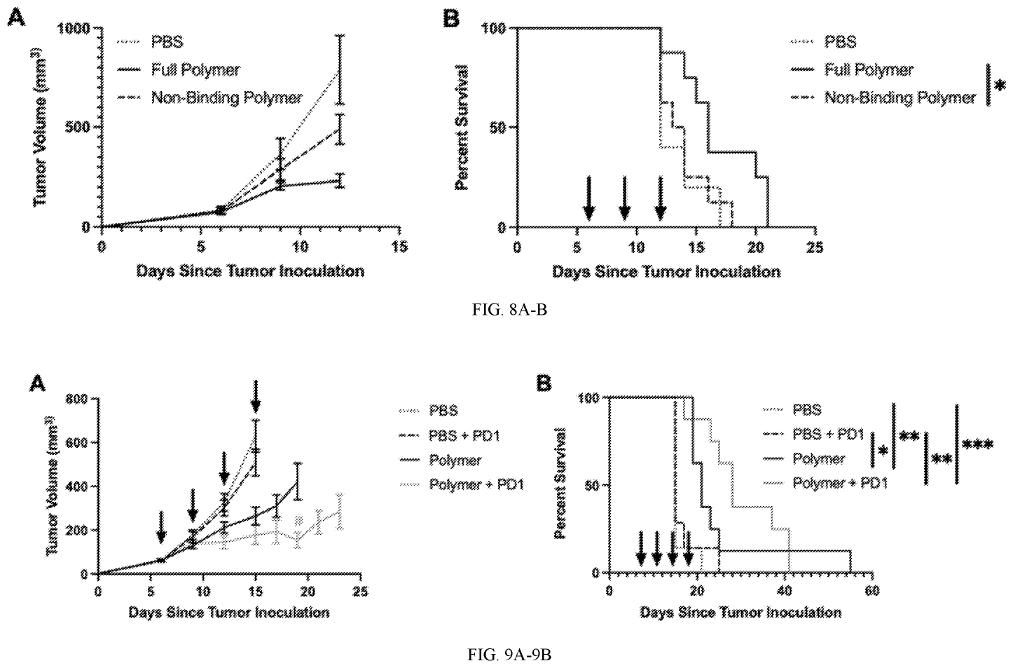

The inventors then evaluated antitumor efficacy of the full p(Man-TLR7-PDS-HPMA) polymer versus a non-binding control polymer p(Man-TLR7). Mice bearing established B16F10 tumors were injected intratumorally with 40 μg TLR7 equivalent (as quantified by absorbance at 327 nm, relative to a standard curve) polymer, either with (full polymer) or without PDS (non-binding), in 30 μL sterile PBS on days 6, 9, and 12 after tumor inoculation. Tumor growth was recorded until the tumor reached a volume of 1000 mm3 and the mice were euthanized (FIG. 8).

10. Antitumor Efficacy—p(Man-TLR7-PDS) Synergizes with Anti-PD-1

The inventors evaluated antitumor efficacy in combination with immune checkpoint inhibitor anti-PD-1 antibody. Mice bearing established CT26 tumors injected intratumorally with 40 μg TLR7 equivalent (as quantified by absorbance at 273 nm, relative to a standard curve) polymer in 30 μL sterile PBS and intraperitoneally with 100 μg anti-PD-1 (29F.1A12, BioXCell) on days 6, 9, 12, and 15 after tumor inoculation. Tumor growth was recorded until the tumor reached a volume of 1000 mm3 and the mice were euthanized (FIG. 9).

Here, the inventors have demonstrated the in vivo efficacy of the PDS platform via the delivery of polymeric TLR7 agonist. When compared directly to a non-binding control, the full p(Man-TLR7-PDS) slows tumor growth and significantly prolongs survival. The polymer also significantly synergizes with checkpoint inhibitor anti-PD-1 in its antitumor efficacy. Together, these data support the hypothesis that the thiol-binding delivery platform can be used to better deliver immunotherapeutic agents to tumor cells.

### B. References

The following references and the publications referred to throughout the specification, to the extent that they provide exemplary procedural or other details supplementary to those set forth herein, are specifically incorporated herein by reference.
1. Haslam, A.; Prasad, V., Estimation of the percentage of US patients with cancer who are eligible for and respond to checkpoint inhibitor immunotherapy drugs. JAMA network open 2019, 2 (5), e192535-e192535.
2. Youn, B.; Trikalinos, N. A.; Mor, V.; Wilson, I. B.; Dahabreh, I. J., Real-world use and survival outcomes of immune checkpoint inhibitors in older adults with non-small cell lung cancer. Cancer 2020, 126 (5), 978-985.
3. Kaczanowska, S.; Joseph, A. M.; Davila, E., TLR agonists: our best frenemy in cancer immunotherapy. Journal of leukocyte biology 2013, 93 (6), 847-863.
4. Lee, S. N.; Jin, S. M.; Shin, H. S.; Lim, Y. T., Chemical Strategies to Enhance the Therapeutic Efficacy of Toll-like Receptor Agonist Based Cancer Immunotherapy. Accounts of Chemical Research 2020, 53 (10), 2081-2093.
5. Guo, C.; Manjili, M. H.; Subjeck, J. R.; Sarkar, D.; Fisher, P. B.; Wang, X.-Y., Therapeutic cancer vaccines: past, present, and future. Advances in cancer research 2013, 119, 421-475.
6. Wilson, D. S.; Hirosue, S.; Raczy, M. M.; Bonilla-Ramirez, L.; Jeanbart, L.; Wang, R.; Kwissa, M.; Franetich, J.-F.; Broggi, M. A.; Diaceri, G., Antigens reversibly conjugated to a polymeric glyco-adjuvant induce protective humoral and cellular immunity. Nature materials 2019, 18 (2), 175-185.
7. Gray, L. T.; Raczy, M. M.; Briquez, P. S.; Marchell, T. M.; Alpar, A. T.; Wallace, R. P.; Volpatti, L. R.; Sasso, M. S.; Cao, S.; Nguyen, M.; Mansurov, A.; Budina, E.; Watkins, E. A.; Solanki, A.; Mitrousis, N.; Reda, J. W.; Yu, S. S.; Tremain, A. C.; Wang, R.; Nicolaescu, V.; Furlong, K.; Dvorkin, S.; Manicassamy, B.; Randall, G.; Wilson, D. S.; Kwissa, M.; Swartz, M. A.; Hubbell, J. A., Generation of potent cellular and humoral immunity against SARS-COV-2 antigens via conjugation to a polymeric glyco-adjuvant. Biomaterials 2021, 278, 121159.
8. Hanahan, D.; Weinberg, R. A., The hallmarks of cancer. cell 2000, 100 (1), 57-70.
9. Gatenby, R. A.; Gillies, R. J., Why do cancers have high aerobic glycolysis? Nature reviews cancer 2004, 4 (11), 891-899.
10. Dirican, N.; Dirican, A.; Sen, O.; Aynali, A.; Atalay, S.; Bircan, H. A.; Oztürk, O.; Erdogan, S.; Cakir, M.; Akkaya, A., Thiol/disulfide homeostasis: A prognostic biomarker for patients with advanced non-small cell lung cancer? Redox Report 2016, 21 (5), 197-203.
11. Eryilmaz, M. A.; Kozanhan, B.; Solak, I.; çetinkaya, ç. D.; Neselioglu, S.; Erel, Ö., Thiol-disulfide homeostasis in breast cancer patients. Journal of cancer research and therapeutics 2019, 15 (5), 1062.
12. Erel, O.; Erdoğan, S., Thiol-disulfide homeostasis: an integrated approach with biochemical and clinical aspects. Turkish journal of medical sciences 2020, 50 (SI-2), 1728-1738.
13. Chaiswing. L.; Bourdeau-Heller, J. M.; Zhong. W.; Oberley. T. D., Characterization of redox state of two human prostate carcinoma cell lines with different degrees of aggressiveness. Free Radical Biology and Medicine 2007, 43 (2), 202-215.
14. Banjac, A.; Perisic, T.; Sato, H.; Seiler, A.; Bannai, S.; Weiss, N.; KöIle, P.; Tschoep, K.; Issels. R.; Daniel, P., The cystine/cysteine cycle: a redox cycle regulating susceptibility versus resistance to cell death. Oncogene 2008, 27 (11), 1618-1628.
15. Ceccarelli, J.; Delfino, L.; Zappia, E.; Castellani, P.; Borghi, M.; Ferrini, S.; Tosetti, F.; Rubartelli, A., The redox state of the lung cancer microenvironment depends on the levels of thioredoxin expressed by tumor cells and affects tumor progression and response to prooxidants. International Journal of Cancer 2008, 123 (8), 1770-1778.
16. Chaiswing. L.; Zhong, W.; Cullen, J. J.; Oberley, L. W.; Oberley, T. D., Extracellular redox state regulates features associated with prostate cancer cell invasion. Cancer research 2008, 68 (14), 5820-5826.
17. Jonas, C. R.; Ziegler, T. R.; Gu, L. H.; Jones, D. P., Extracellular thiol/disulfide redox state affects proliferation rate in a human colon carcinoma (Caco2) cell line. Free Radical Biology and Medicine 2002, 33 (11), 1499-1506.
18. Menchise, V.; Digilio, G.; Gianolio, E.; Cittadino, E.; Catanzaro, V.; Carrera, C.; Aime, S., In vivo labeling of B16 melanoma tumor xenograft with a thiol-reactive gadolinium based MRI contrast agent. Molecular pharmaceutics 2011, 8 (5), 1750-1756.
19. Digilio, G.; Catanzaro, V.; Fedeli, F.; Gianolio, E.; Menchise, V.; Napolitano, R.; Gringeri, C.; Aime, S., Targeting exofacial protein thiols with Gd III complexes. An efficient procedure for MRI cell labelling. Chemical communications 2009, (8), 893-895.
20. Digilio, G.; Menchise, V.; Gianolio, E.; Catanzaro, V.; Carrera, C.; Napolitano, R.; Fedeli, F.; Aime, S., Exofacial protein thiols as a route for the internalization of Gd (III)-based complexes for magnetic resonance imaging cell labeling. Journal of medicinal chemistry 2010, 53 (13), 4877-4890.
21. Altinbasak, I.; Arslan, M.; Sanyal, R.; Sanyal, A., Pyridyl disulfide-based thiol-disulfide exchange reaction: shaping the design of redox-responsive polymeric materials. Polymer Chemistry 2020.
22. Bontempo, D.; Heredia, K. L.; Fish, B. A.; Maynard, H. D., Cysteine-reactive polymers synthesized by atom transfer radical polymerization for conjugation to proteins. Journal of the American Chemical Society 2004, 126 (47), 15372-15373.
23. Vanparijs, N.; Maji, S.; Louage, B.; Voorhaar, L.; Laplace, D.; Zhang, Q.; Shi, Y.; Hennink, W. E.; Hoogenboom, R.; De Geest, B., Polymer-protein conjugation via a ‘grafting to’ approach—a comparative study of the performance of protein-reactive RAFT chain transfer agents. Polymer Chemistry 2015, 6 (31), 5602-5614.
24. Liu, J.; Liu, H.; Bulmus, V.; Tao, L.; Boyer, C.; Davis, T. P., A simple methodology for the synthesis of heterotelechelic protein-polymer-biomolecule conjugates. Journal of Polymer Science Part A: Polymer Chemistry 2010, 48 (6), 1399-1405.
25. Vázquez-Dorbatt, V.; Tolstyka, Z. P.; Chang, C.-W.; Maynard, H. D., Synthesis of a pyridyl disulfide end-functionalized glycopolymer for conjugation to biomolecules and patterning on gold surfaces. Biomacromolecules 2009, 10 (8), 2207-2212.
26. Gunasekaran, K.; Nguyen, T. H.; Maynard, H. D.; Davis, T. P.; Bulmus, V., Conjugation of siRNA with Comb-Type PEG Enhances Serum Stability and Gene Silencing Efficiency. Macromolecular rapid communications 2011, 32 (8), 654-659.
27. El-Sayed, M. E.; Hoffman, A. S.; Stayton, P. S., Rational design of composition and activity correlations for pH-sensitive and glutathione-reactive polymer therapeutics. Journal of Controlled Release 2005, 101 (1-3), 47-58.
28. KC. R. B.; Thapa, B.; Xu, P., pH and redox dual responsive nanoparticle for nuclear targeted drug delivery. Molecular pharmaceutics 2012, 9 (9), 2719-2729.
29. Smits, E. L.; Ponsaerts, P.; Berneman, Z. N.; Van Tendeloo, V. F., The use of TLR7 and TLR8 ligands for the enhancement of cancer immunotherapy. The oncologist 2008, 13 (8), 859-875.
30. Chi, H.; Li, C.; Zhao, F. S.; Zhang. L.; Ng. T. B.; Jin, G.; Sha, O., Anti-tumor activity of toll-like receptor 7 agonists. Frontiers in pharmacology 2017, 8, 304.
31. Krieg, A. M., Development of TLR9 agonists for cancer therapy. The Journal of clinical investigation 2007, 117 (5), 1184-1194.
32. Krieg. A. M., Toll-like receptor 9 (TLR9) agonists in the treatment of cancer. Oncogene 2008, 27 (2), 161-167.
33. Shirota, H.; Tross, D.; Klinman, D. M., CpG oligonucleotides as cancer vaccine adjuvants. Vaccines 2015, 3 (2), 390-407.
34. Jung. I. D.; Jeong. S. K.; Lee, C.-M.; Noh, K. T.; Heo, D. R.; Shin, Y. K.; Yun, C.-H.; Koh, W.-J.; Akira, S.; Whang, J., Enhanced efficacy of therapeutic cancer vaccines produced by co-treatment with Mycobacterium tuberculosis heparin-binding hemagglutinin, a novel TLR4 agonist. Cancer Research 2011, 71 (8), 2858-2870.
35. Mauldin, I. S.; Wang, E.; Deacon, D. H.; Olson, W. C.; Bao, Y.; Slingluff Jr, C. L., TLR 2/6 agonists and interferon-gamma induce human melanoma cells to produce CXCL 10. International journal of cancer 2015, 137 (6), 1386-1396.
36. Napolitani, G.; Rinaldi, A.; Bertoni, F.; Sallusto, F.; Lanzavecchia, A., Selected Toll-like receptor agonist combinations synergistically trigger a T helper type 1-polarizing program in dendritic cells. Nature immunology 2005, 6 (8), 769-776.
37. Tom, J. K.; Dotsey, E. Y.; Wong, H. Y.; Stutts, L.; Moore, T.; Davies, D. H.; Felgner, P. L.; Esser-Kahn, A. P., Modulation of innate immune responses via covalently linked TLR agonists. ACS central science 2015, 1 (8), 439-448.
38. Chen, B.-K.; Lo, S.-H.; Lee, S.-F., Temperature responsive methacrylamide polymers with antibacterial activity. Chinese Journal of Polymer Science 2010, 28 (4), 607-613.

### Example 2—Cysteine Binding Pattern Recognition Receptor Agonists for Intratumoral Immunotherapy

Immune stimulating adjuvants can powerfully trigger the innate immune system and initiate a robust antitumor response (1). However, their systemic administration can lead to significant off-target effects (2). For that reason, topical administration and intratumoral injection are the preferred route of administration of some of these adjuvants. For example, TLR7/8 agonist imiquimod is applied topically in clinic (3), and TLR9 agonist CpG has long been under investigation as an intratumorally injected adjuvant (4). Even then, many adjuvants are too powerful and elicit systemic toxicity (5, 6). To better direct the inflammatory immune response towards tumor antigens, the inventors developed a multivalent, thiol-binding adjuvant delivery platform that binds unpaired cysteines on tumor cell surface proteins and debris. The platform exploits the characteristic metabolic and enzymatic dysregulation of solid tumors (7, 8) to covalently link adjuvant and agonist in an in situ cancer vaccine, while limiting systemic toxicity through intratumoral retention.

Now, the inventors take the concept of cysteine-mediated delivery and apply it to monomeric small molecule adjuvants, rather than a multivalent polymeric context. Small molecules are promising due to high membrane permeability and low batch-to-batch variability (9, 10). The inventors are interested in applying this principle to small molecule pattern recognition receptor (PRR) agonists with known antitumor efficacy. PRRs broadly include Toll-like receptors (TLR), NOD-like receptors (NLR) and related NLRP, RIG-I-like receptors (RLRs), and stimulators of interferon genes (STING) which respond to viral and bacterial pathogen- and damage-associated molecular patterns (PAMPs, DAMPs). Activation of these receptors leads to an inflammatory response which can be directed for tumor-specific immunity (11, 12). Many of these agonists are unstable, macromolecular, infectious material, such as single stranded RNA in the case of TLR7 and TLR8. However, small molecule agonists have been characterized for many receptors—examples of other known small molecule PRR agonists are given in Table 1.

TABLE 1
Clinically relevant small molecule PRR agonists.

| Target Receptor | Agonist Molecule |
|---|---|
| TLR1/2 | CU-T12-9 |
| TLR1/2 | SMU127 |
| TLR4 | Paclitaxil |
| TLR4 | 1Z105 |
| TLR7 | R848 |
| TLR7/8 | 3M-052 |
| TLR8 (and NLRP3) | VTX-2337 |
| NLRP3 | BMS-986299 |
| NOD-2 | MDP |
| RLR | KIN1408 |
| STING | SR-717 |
| STING | MSA-2 |

The molecules the inventors aim to develop have three main components: (1) the small molecule adjuvant; (2) a flexible linker consisting of a hydrocarbon chain, poly(ethylene glycol) (PEG) repeats, or a combination of the two; and (3) a thiol-reactive moiety of either pyridyl disulfide (PDS) or maleimide. Examples of these structures are shown in Scheme 1.

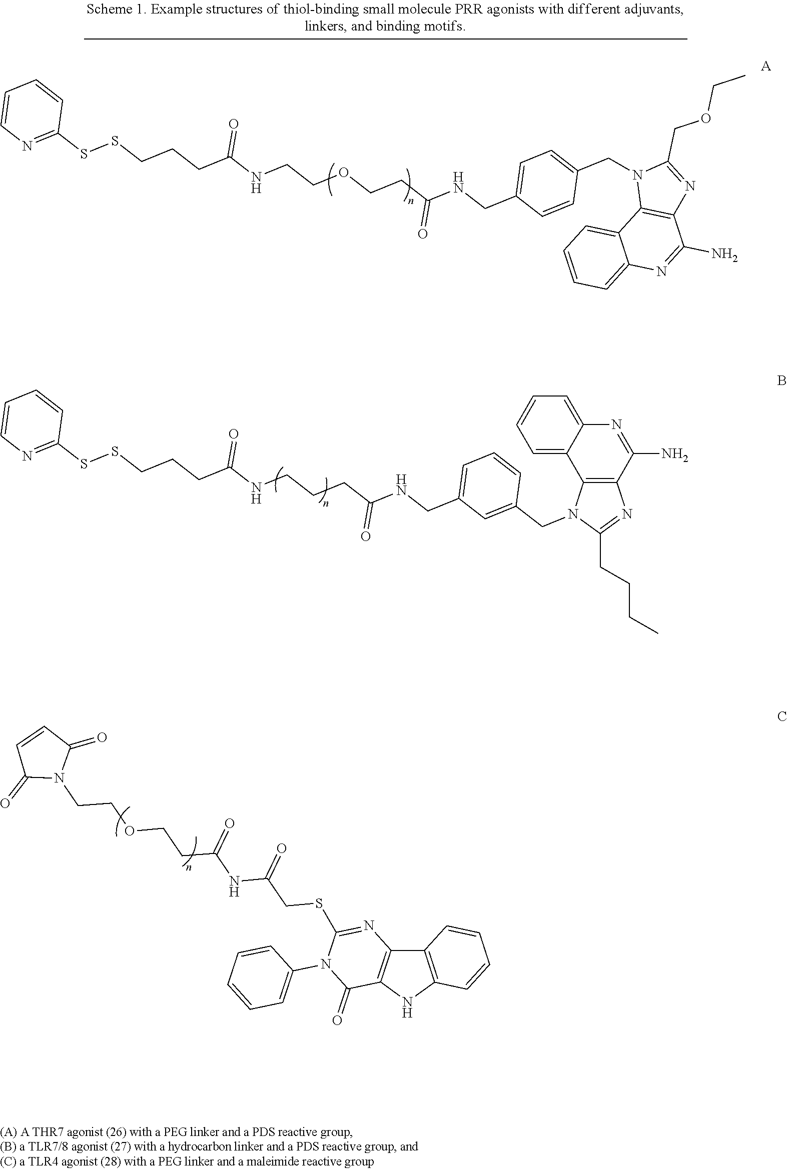

### REFERENCES

The following references and the publications referred to throughout the specification, to the extent that they provide exemplary procedural or other details supplementary to those set forth herein, are specifically incorporated herein by reference.

### REFERENCES

1. Hu, H.-G.; Li, Y.-M., Emerging adjuvants for cancer immunotherapy. Frontiers in Chemistry 2020, 8, 601.
2. Taniguchi, K.; Karin, M., NF-κB, inflammation, immunity and cancer: coming of age. Nature Reviews Immunology 2018, 18 (5), 309-324.
3. Vacchelli, E.; Galluzzi, L.; Eggermont, A.; Fridman, W. H.; Galon, J.; Sautès-Fridman, C.; Tartour, E.; Zitvogel, L.; Kroemer, G., Trial watch: FDA-approved Toll-like receptor agonists for cancer therapy. Oncoimmunology 2012, 1 (6), 894-907.
4. Shirota, Y.; Shirota, H.; Klinman, D. M., Intratumoral injection of CpG oligonucleotides induces the differentiation and reduces the immunosuppressive activity of myeloid-derived suppressor cells. The Journal of Immunology 2012, 188 (4), 1592-1599.
5. Varshney, D.; Qiu, S. Y.; Graf, T. P.; McHugh, K. J., Employing drug delivery strategies to overcome challenges using TLR7/8 agonists for cancer immunotherapy. The AAPS Journal 2021, 23 (4), 1-18.
6. Lee, S. N.; Jin, S. M.; Shin, H. S.; Lim, Y. T., Chemical Strategies to Enhance the Therapeutic Efficacy of Toll-like Receptor Agonist Based Cancer Immunotherapy. Accounts of Chemical Research 2020, 53 (10), 2081-2093.
7. Hanahan, D., Hallmarks of Cancer: New Dimensions. Cancer Discovery 2022, 12 (1), 31-46.
8. Fukumura, D.; Jain, R. K., Tumor microenvironment abnormalities: causes, consequences, and strategies to normalize. Journal of Cellular Biochemistry 2007, 101 (4), 937-949.
9. van der Zanden, S. Y.; Luimstra, J. J.; Neefjes, J.; Borst, J.; Ovaa, H., Opportunities for small molecules in cancer immunotherapy. Trends in immunology 2020, 41 (6), 493-511.
10. Zhang. J.; Zhang, Y.; Qu. B.; Yang, H.; Hu, S.; Dong, X., If small molecules immunotherapy comes, can the prime be far behind? European Journal of Medicinal Chemistry 2021, 218, 113356.
11. Zhu, G.; Xu, Y.; Cen, X.; Nandakumar, K. S.; Liu, S.; Cheng, K., Targeting pattern-recognition receptors to discover new small molecule immune modulators. European Journal of Medicinal Chemistry 2018, 144, 82-92.
12. Xu, J.; Li, X.; Du, Y., Antibody-Pattern Recognition Receptor Agonist Conjugates: A Promising Therapeutic Strategy for Cancer. Advanced Biology 2022, 6 (3), 2101065.
13. Cheng. K.; Gao, M.; Godfroy, J. I.; Brown, P. N.; Kastelowitz, N.; Yin, H., Specific activation of the TLR1-TLR2 heterodimer by small-molecule agonists. Science advances 2015, 1 (3), e1400139.
14. Chen, Z.; Cen, X.; Yang. J.; Tang. X.; Cui, K.; Cheng, K., Structure-based discovery of a specific TLR1-TLR2 small molecule agonist from the ZINC drug library database. Chemical Communications 2018, 54 (81), 11411-11414.
15. Wanderley, C. W.; Colon, D. F.; Luiz, J. P. M.; Oliveira, F. F.; Viacava, P. R.; Leite, C. A.; Pereira, J. A.; Silva, C. M.; Silva, C. R.; Silva, R. L., Paclitaxel reduces tumor growth by reprogramming tumor-associated macrophages to an M1 profile in a TLR4-dependent manner. Cancer research 2018, 78 (20), 5891-5900.
16. Chan, M.; Kakitsubata, Y.; Hayashi, T.; Ahmadi, A.; Yao, S.; Shukla, N. M.; Oyama, S.-y.; Baba, A.; Nguyen, B.; Corr, M., Structure-activity relationship studies of pyrimido[5,4-b]indoles as selective Toll-like receptor 4 ligands. Journal of medicinal chemistry 2017, 60 (22), 9142-9161.
17. Chan, M.; Hayashi, T.; Mathewson, R. D.; Nour, A.; Hayashi, Y.; Yao, S.; Tawatao, R. I.; Crain, B.; Tsigelny, I. F.; Kouznetsova, V. L., Identification of substituted pyrimido[5,4-b]indoles as selective Toll-like receptor 4 ligands. Journal of medicinal chemistry 2013, 56 (11), 4206-4223.
18. Michaelis, K. A.; Norgard, M. A.; Zhu, X.; Levasseur, P. R.; Sivagnanam, S.; Liudahl, S. M.; Burfeind, K. G.; Olson, B.; Pelz, K. R.; Ramos, D. M. A., The TLR7/8 agonist R848 remodels tumor and host responses to promote survival in pancreatic cancer. Nature Communications 2019, 10 (1), 1-15.
19. Zhao, B. G.; Vasilakos, J. P.; Tross, D.; Smirnov, D.; Klinman, D. M., Combination therapy targeting toll like receptors 7, 8 and 9 eliminates large established tumors. Journal for immunotherapy of cancer 2014, 2 (1), 1-10.
20. Dietsch, G. N.; Lu, H.; Yang, Y.; Morishima, C.; Chow, L. Q.; Disis, M. L.; Hershberg. R. M., Coordinated activation of toll-like receptor8 (TLR8) and NLRP3 by the TLR8 agonist, VTX-2337, ignites tumoricidal natural killer cell activity. PloS one 2016, 11 (2), e0148764.
21. Ma, X.; Qiu. Y.; Sun, Y.; Zhu, L.; Zhao, Y.; Li. T.; Lin, Y.; Ma, D.; Qin, Z.; Sun, C., NOD2 inhibits tumorigenesis and increases chemosensitivity of hepatocellular carcinoma by targeting AMPK pathway. Cell death & disease 2020, 11 (3), 1-12.
22. Pattabhi, S.; Wilkins, C. R.; Dong, R.; Knoll, M. L.; Posakony, J.; Kaiser, S.; Mire, C. E.; Wang, M. L.; Ireton, R. C.; Geisbert, T. W., Targeting innate immunity for antiviral therapy through small molecule agonists of the RLR pathway. Journal of virology 2015, 90 (5), 2372-2387.
23. Elion, D. L.; Cook, R. S., Harnessing RIG-I and intrinsic immunity in the tumor microenvironment for therapeutic cancer treatment. Oncotarget 2018, 9 (48), 29007.
24. Chin, E. N.; Yu, C.; Vartabedian, V. F.; Jia, Y.; Kumar, M.; Gamo, A. M.; Vernier, W.; Ali, S. H.; Kissai, M.; Lazar, D. C., Antitumor activity of a systemic STING-activating non-nucleotide cGAMP mimetic. Science 2020, 369 (6506), 993-999.
25. Pan, B.-S.; Perera, S. A.; Piesvaux, J. A.; Presland, J. P.; Schroeder, G. K.; Cumming, J. N.; Trotter, B. W.; Altman, M. D.; Bucvich, A. V.; Cash, B., An orally available non-nucleotide STING agonist with antitumor activity. Science 2020, 369 (6506), caba6098.
26. Wilson, D. S.; Hirosue, S.; Raczy, M. M.; Bonilla-Ramirez, L.; Jeanbart, L.; Wang, R.; Kwissa, M.; Franetich, J.-F.; Broggi, M. A.; Diaceri, G., Antigens reversibly conjugated to a polymeric glyco-adjuvant induce protective humoral and cellular immunity. Nature Materials 2019, 18 (2), 175-185.
27. Ganapathi, L.; Van Haren, S.; Dowling, D. J.; Bergelson, I.; Shukla, N. M.; Malladi, S. S.; Balakrishna, R.; Tanji, H.; Ohto, U.; Shimizu, T., The imidazoquinoline toll-like receptor-7/8 agonist hybrid-2 potently induces cytokine production by human newborn and adult leukocytes. PLOS One 2015, 10 (8), c0134640.
28. Tom. J. K.; Dotsey, E. Y.; Wong, H. Y.; Stutts. L.; Moore, T.; Davies, D. H.; Felgner, P. L.; Esser-Kahn, A. P., Modulation of innate immune responses via covalently linked TLR agonists. ACS central science 2015, 1 (8), 439-448.

### Example 3: Tumor Cell-Surface Binding of Immune-Stimulating Polymeric Glyco-Adjuvant Via Cysteine-Reactive Pyridyl Disulfide Promotes Antitumor Immunity

Immune stimulating agents like Toll-like receptor 7 (TLR7) agonists induce potent antitumor immunity but are limited in their therapeutic window due to off-target immune activation. Here, the inventors developed a polymeric delivery platform that binds excess unpaired cysteines on tumor cell surfaces and debris to adjuvant tumor neoantigens as an in situ vaccine. The metabolic and enzymatic dysregulation in the tumor microenvironment produces these exofacial free thiols, which can undergo efficient disulfide exchange with thiol-reactive pyridyl disulfide moieties upon intratumoral injection. These functional monomers are incorporated into a co-polymer with pendant mannose groups and TLR7 agonists to target both antigen and adjuvant to antigen presenting cells. When tethered in the tumor, the polymeric glyco-adjuvant induces a robust antitumor response, slowing tumor growth and prolonging survival of B16F10 tumor-bearing mice and other murine cancer models. The construct additionally reduces systemic toxicity associated with clinically-relevant small molecule TLR7 agonists.

### A. Introduction

While the features of solid tumors vary considerably, some physiological properties are common and create a characteristic microenvironment for tumor growth and proliferation (1). Rapid, disordered metabolism leads to a harsh environment with unique chemical characteristics including hypoxia and acidosis (2, 3). This also leads to physical disorganization in the form of disordered and hyperpermeable vasculature and interstitial hypertension (4). For rapid growth and immune evasion to occur, cancer cells make adaptive changes to protein expression, especially to those involved in metabolism and extracellular matrix degradation, including p53, hypoxia-inducible factor-1α, and matrix metalloproteases (5, 6). Together, these create a characteristic tumor microenvironment.

While the advent of immune checkpoint inhibitors has transformed the field of cancer immunotherapy, their suboptimal response rate in certain tumors, including melanoma and breast cancer, leaves room for the development of complementary therapeutics (7-9). Immune stimulating agonists such as Toll-like receptor (TLR) agonists can activate the innate immune system and potentiate antitumor immunity (10, 11). In particular, the inventors are interested in the delivery of agonists to TLR7 and TLR8, whose natural ligand is single-stranded RNA (12). Improving the tolerability and efficacy of such agonists using chemical modifications to localize them in the tumor microenvironment can broaden their clinical applicability.

Given the dysregulated metabolism of the tumor microenvironment, the inventors hypothesized that the metabolic stress of tumor growth produces an excess of unpaired cysteines on cell surface proteins relative to other cells of the body. The inventors envisioned exploiting excess free thiols in the tumor microenvironment for a drug delivery application. Consequently, the inventors created a delivery platform using a thiol-specific reactive moiety, pyridyl disulfide, for conjugation to the tumor cell surface to achieve prolonged tumor retention. The inventors chose to deliver a polymeric glyco-adjuvant previously developed in the inventors' laboratory, p(Man-TLR7), which showed strong efficacy as a vaccinal adjuvant (22). The inventors now pivoted to an immuno-oncology application to adjuvant the tumor cell surface and the neoantigens contained therein and demonstrated that disulfide binding upon intratumoral injection creates an in situ cancer vaccine, inducing strong therapeutic antitumor immunity in monotherapy and in combination therapy with checkpoint inhibition.

### B. Results

### 1. Multivalent, Cysteine-Reactive Polymers can be Synthesized Via PET-RAFT

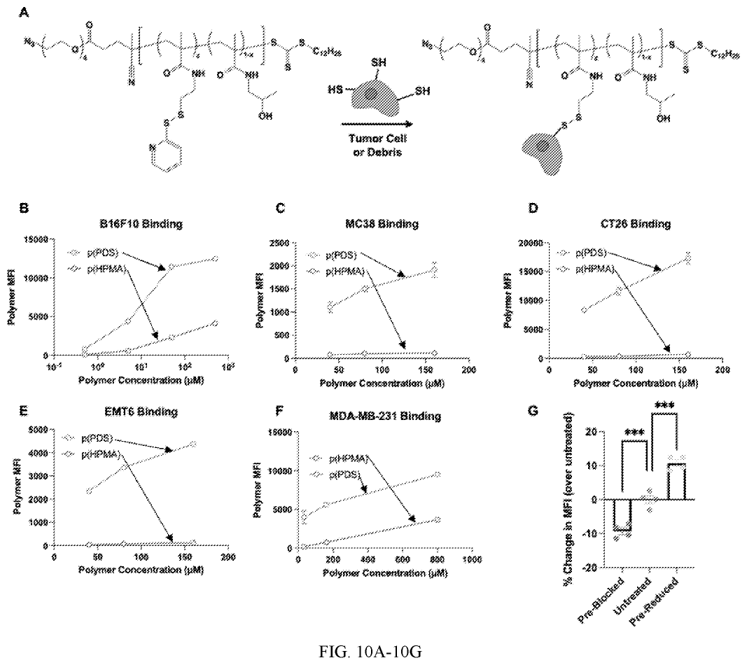

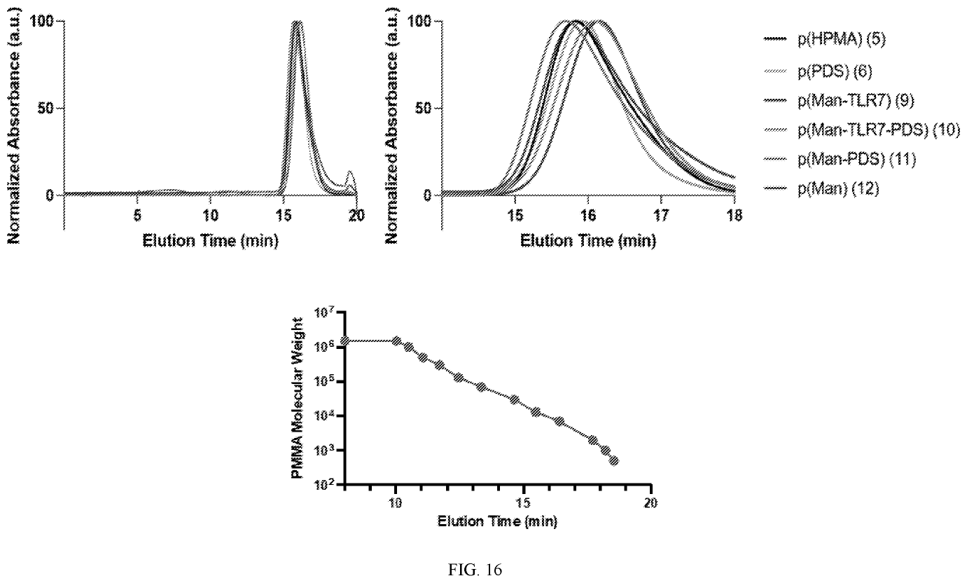

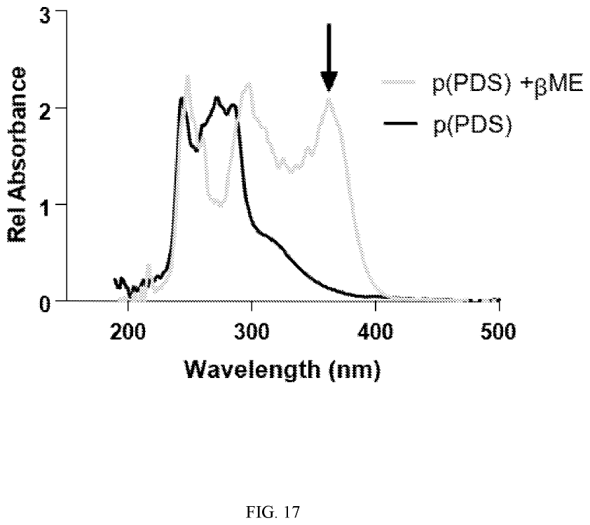

Pyridyl disulfide (PDS) moieties are useful in the drug delivery field due to their rapid and efficient thiol-disulfide exchange reaction, which can be used to reversibly conjugate therapeutics or fabricate materials for in situ binding (23-25). The inventors developed a synthesis scheme for a PDS-containing methacrylamide monomer (26). In order to attain high conversion polymerization without significant crosslinking, the inventors employed photoinduced electron/energy transfer-reversible addition-fragmentation chain transfer (PET-RAFT). This method uses a photoredox catalyst, eosin Y in this case, to initiate controlled free radical polymerization with high conversion and low dispersity (27,28). The inventors co-polymerized the PDS monomer with the bioinert, water-soluble, “spacer” monomer 2-hydroxypropyl methacrylamide (HPMA). This strategy predictably yields statistical copolymers (referred to as p(PDS)) of desired molecular weight and composition (FIG. 16). These functional polymers can react efficiently with unpaired cysteines on tumor cell surface proteins (FIG. 10a). In order to confirm the functional activity of p(PDS), the inventors reduced the polymer with β-mercaptoethanol and monitored the production of the leaving group, 2-mercaptopyridine, which has a characteristic absorbance at 343 nm (29) (FIG. 17).

### 2. Pyridyl Disulfide Mediates Cell Surface Binding and Tumor Retention

Once the inventors confirmed the production of uniform, reactive, PDS-containing polymers, the inventors validated their binding ability to tumor cells in vitro. After incubation at 4° C. to inhibit endocytosis, the inventors were able to confirm, via flow cytometry, that p(PDS) bound B16F10 melanoma cells significantly better than a molecular weight-matched p(HPMA) (non-binding control) (FIG. 10b). This result was validated using other murine cancer cell lines, including MC38 and CT26 colon carcinoma and EMT6 mammary carcinoma (FIG. 10c-e) and human breast cancer cell line MDA-MB-231 (FIG. 10f). In order to confirm that covalent binding to exofacial protein thiols mediated the increase in binding, the inventors first pre-treated B16F10 cells with N-ethyl maleimide to block free thiols or tris(2-carboxyethyl) phosphine (TCEP) to reduce disulfide bonds into free thiols before the polymer incubation. As expected, pre-blocking thiols prevented polymer binding and reduced mean fluorescence intensity (MFI) (FIG. 10g). Likewise, pre-reducing thiols increased reactive sites for polymer binding and thus increased MFI relative to the untreated samples.

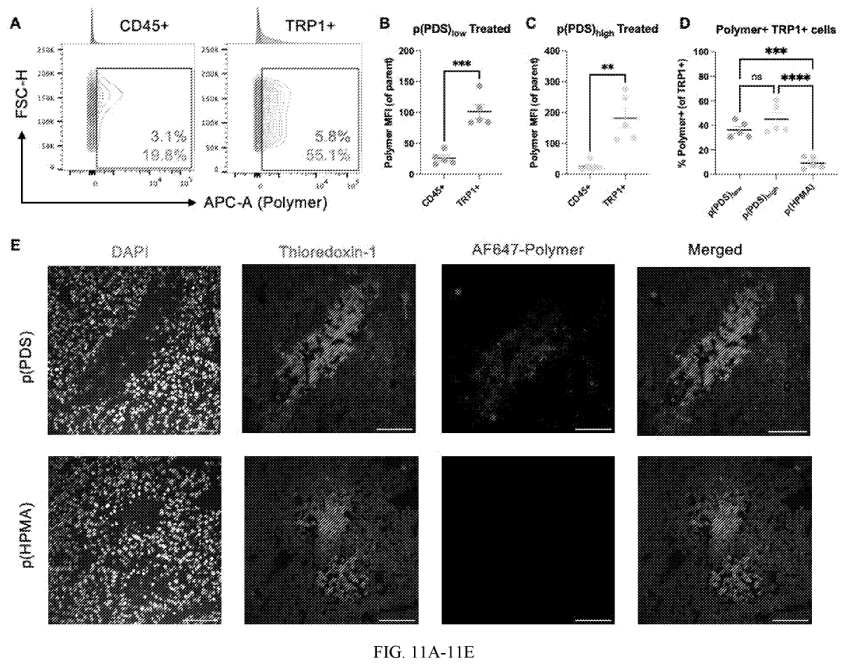

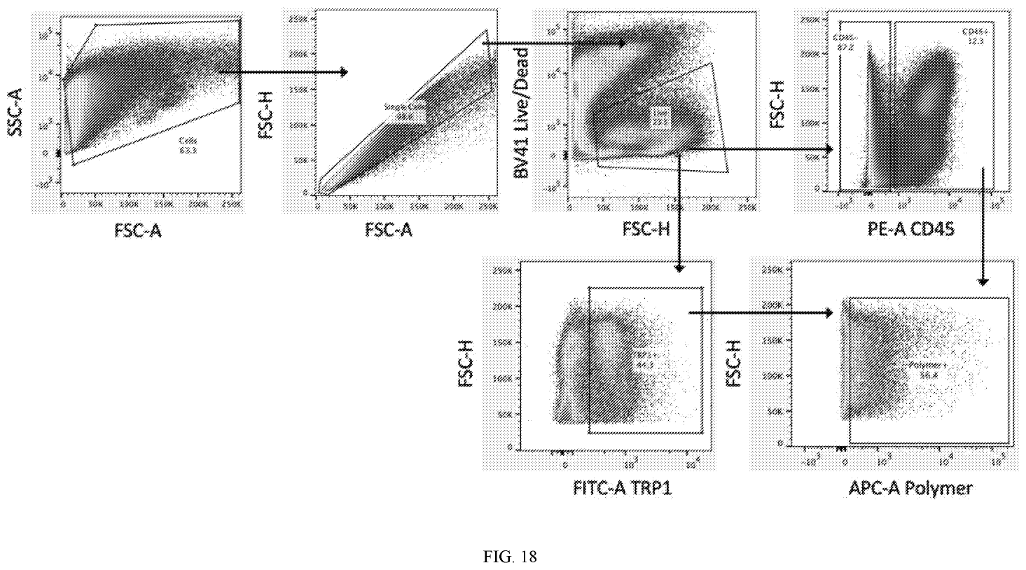

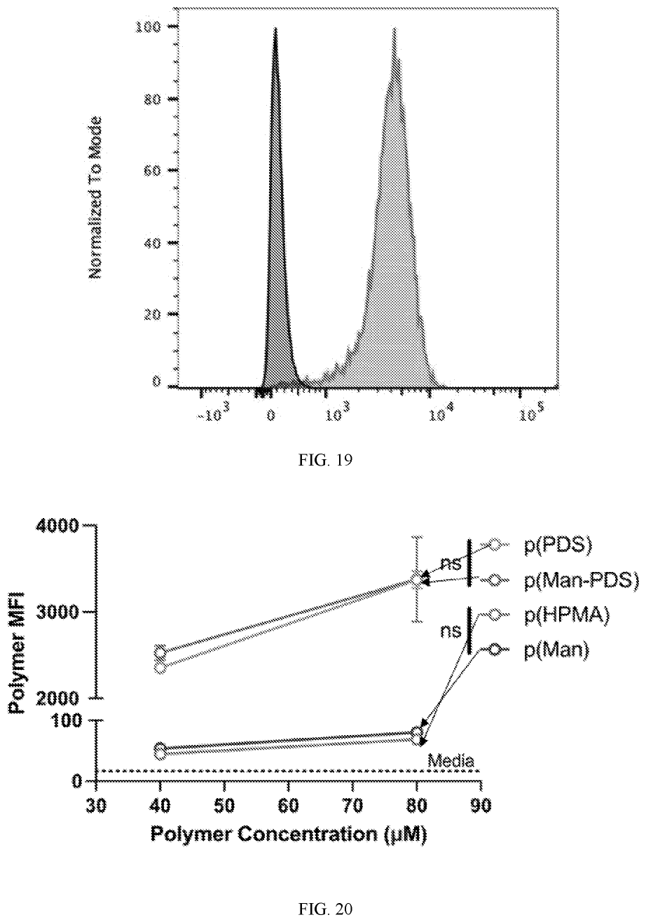

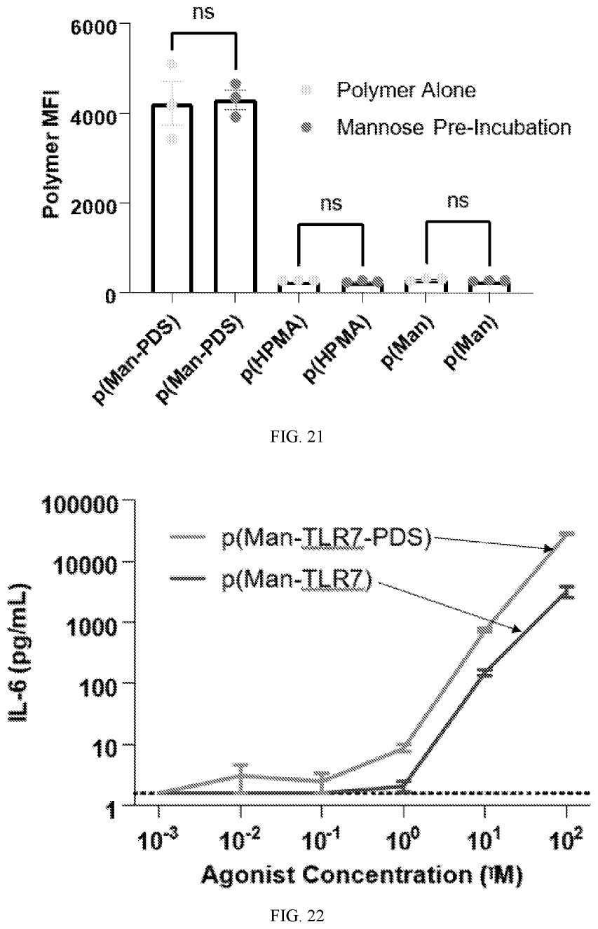

Binding was next confirmed in vivo by two methods. First, the inventors quantified binding to different cell populations after intratumoral injection into B16F10 melanoma-bearing mice. The inventors observed that TRP1⁺ B16F10 cells on average bound more polymer than CD45⁺ immune cells. This result was consistent between mice treated with polymer with low or high PDS content (FIG. 11a-c, FIG. 18). Additionally, both PDS polymers were more likely to bind TRP1⁺ tumor cells than was the non-binding, HPMA-only control, as evidenced by a higher frequency of polymer⁺ cells (FIG. 11d). The inventors also confirmed intratumoral retention via fluorescence microscopy (FIG. 11e). Six hours after injection of fluorescently labeled polymer, the inventors observed retention of p(PDS) but not the non-binding control p(HPMA). Interestingly, the polymer co-localizes with thioredoxin-1, which is a small protein with a redox-active disulfide/dithiol that plays a vital role in redox signaling by reducing oxidized cysteine residues and cleaving disulfide bonds (30). Thioredoxin has been reported to be upregulated many aggressive human cancer types and its expression correlates to worse patient outcomes (31, 32). Thioredoxin-1 staining was particularly strong in regions with low viability (as assessed by DAPI staining), suggesting that intracellular thioredoxin-1, which is detectable in B16F10 (FIG. 19), may be released through cell death. This result suggests thioredoxin-1 upregulation facilitates intratumoral polymer binding.

3. PDS Incorporation Facilitates Tumor Cell Binding and Maintains In Vitro Activity of p(Man-TLR7)

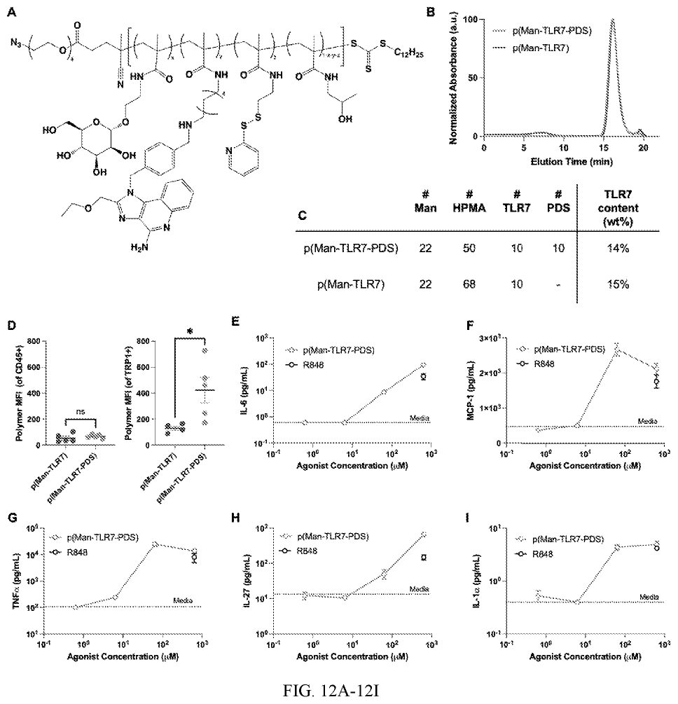

The inventors' lab previously reported a polymeric glyco-adjuvant, p(Man-TLR7), which elicits robust humoral and cellular immunity in a vaccine context (22, 33); the polymer comprises a mannosylated methacrylamide monomer for binding to mannose receptor on antigen-presenting cells (APCs) and triggering endocytosis and an imidazoquinoline TLR7 agonist-conjugated methacrylamide monomer for APC activation once inside the endosome. The inventors now evaluated the antitumor efficacy of the glyco-adjuvant inspired by the success of other TLR7 agonists in cancer immunotherapy (34, 35). To test the efficacy of the delivery platform, the inventors incorporated PDS monomers into p(Man-TLR7) to produce a thiol-binding polymeric glyco-adjuvant, p(Man-TLR7-PDS) (FIG. 12a). For this polymer, the inventors selected feed ratios of the TLR7 and mannose monomers such that the weight percent of the TLR7 agonist would be 15%, which was previously confirmed to be optimally active while still being water-soluble (22). Low dispersity, uniform polymers with similar size were synthesized via PET-RAFT (FIG. 12b). The PDS monomer ratio was comparable to the p(PDS) high polymer from binding studies (FIG. 12c).

Because the functional polymer, p(Man-TLR7-PDS) could also hypothetically bind mannose receptors or other c-type lectins on tumor cell surfaces (36, 37), the inventors verified that mannose monomer incorporation into the polymer did not affect binding, with or without mannose pre-incubation (FIG. 20,21). Thus, the full immune-functional co-polymer also binds tumor cell surfaces in an exclusively thiol-dependent manner. Further, the inventors replicated the in vivo cell binding flow cytometry experiment (FIG. 11a-c, FIG. 18) to determine whether mannose and TLR7 agonist incorporation affected PDS-mediated cell binding and uptake. These results were consistent with the previous study and demonstrated low polymer binding to CD45⁺ immune cells and significantly higher binding of only PDS-containing p(Man-TLR7-PDS) to TRP1⁺ B16F10 tumor cells (FIG. 12d).

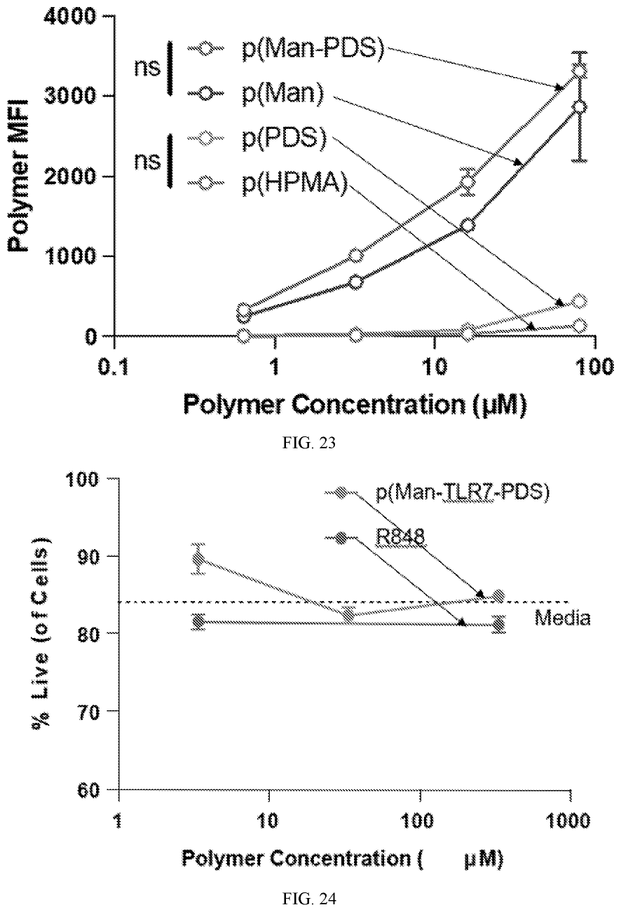

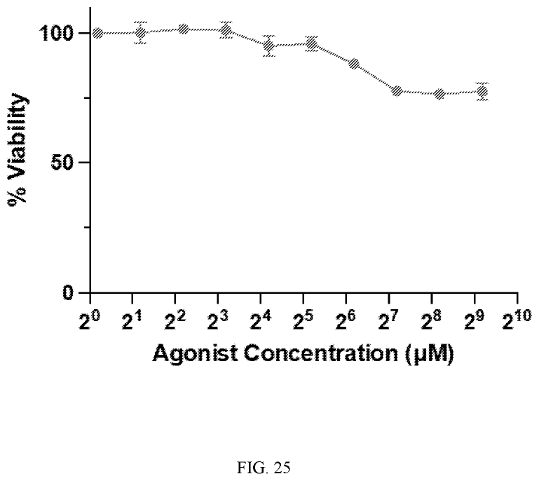

Previous work with p(Man-TLR7) focused on in vitro activity on murine bone marrow derived dendritic cells (BMDCs) (FIG. 22). Here, to demonstrate immune agonization by the co-polymer, the inventors used macrophages as a model, also noting the high frequency tumor-associated macrophages expressing macrophage mannose receptor CD206 (38, 39). The inventors stimulated macrophage-like RAW 264.7 cells and observed a dose-dependent increase in pro-inflammatory cytokines that are downstream of TLR7 signaling (FIG. 12e-i) (40). As a positive control for the assay, the inventors also included a well-known TLR7/8 agonist R848 at the top concentration. The inventors also confirmed that polymer uptake by antigen presenting cells is mediated by mannose, not PDS, using a BMDC uptake experiment (FIG. 23). This can be explained by the high affinity of mannose for CD206 and other mannose receptors or by the relative lack of unpaired cysteines on non-tumor cell surfaces. The inventors also verified the polymer did not have inherent cytotoxicity to BMDCs or B16F10 melanoma cells (FIG. 24,25).

4. p(Man-TLR7-PDS) Significantly Slows Tumor Growth in Orthotopic Murine Cancers

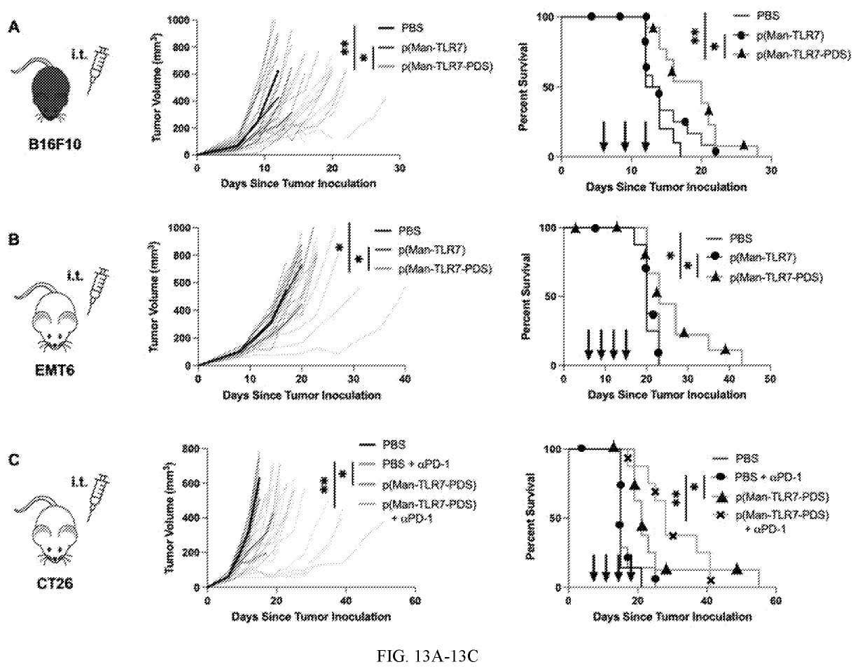

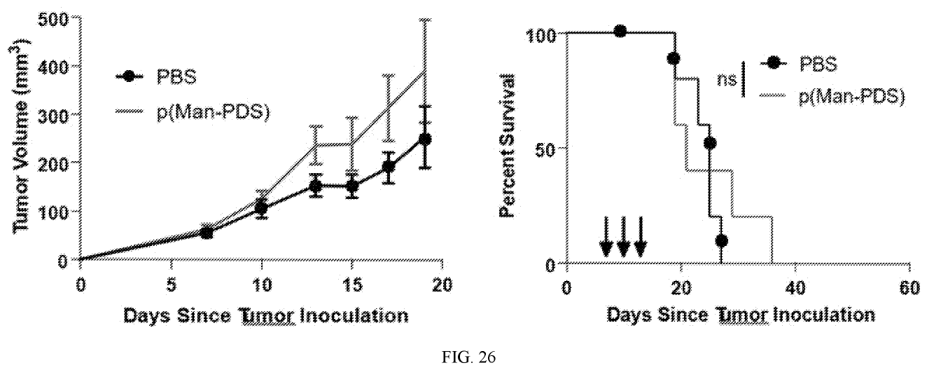

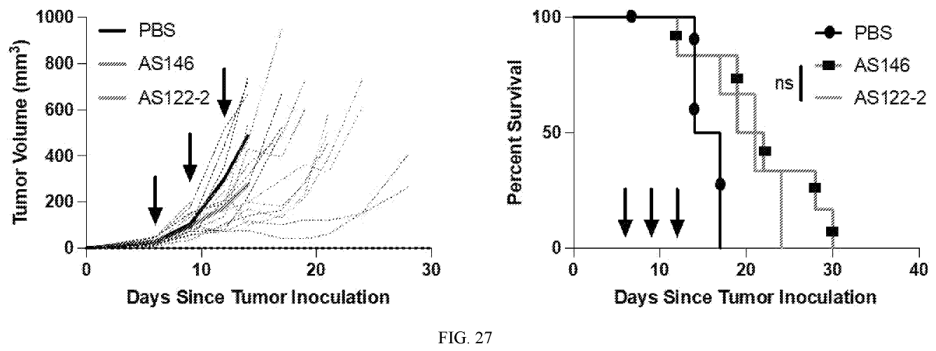

To evaluate the superiority of the thiol-binding p(Man-TLR7-PDS) over the non-binding p(Man-TLR7), the inventors injected B16F10 melanoma-bearing mice intratumorally with the polymers, with the dose based on TLR7 content. The inventors observed slowing of tumor growth and prolongation of survival in mice treated with the binding polymer (FIG. 13a). These results were also confirmed in orthotopic EMT6, an immune-excluded triple-negative breast cancer model (FIG. 13b), demonstrating the broad applicability of the tumor-agnostic delivery platform. This supports the hypothesis that PDS-mediated disulfide anchoring can help increase the antitumor efficacy of cancer immunotherapies. As a control, the inventors also studied the antitumor efficacy of non-adjuvanted p(Man-PDS) (FIG. 26). The polymer provided no tumor protection, verifying that the antitumor efficacy of the polymer is TLR7 agonist dependent. Additionally, the inventors compared the antitumor efficacy of two fully separate batches of p(Man-TLR7-PDS) to ensure low batch-to-batch variability (FIG. 27).

5. p(Man-TLR7-PDS) Improves Efficacy of Checkpoint Inhibitors

To demonstrate that this technology can enhance the efficacy of immune checkpoint blockade therapeutics, the inventors evaluated its efficacy in combination with the immune checkpoint inhibitor anti-PD-1, which is the most commonly used form of immunotherapy (41). The inventors selected CT26 colon carcinoma as the disease model due to its documented low-to-moderate response rate to checkpoint inhibitors (42). In this model, the inventors dosed p(Man-TLR7-PDS) intratumorally and anti-PD-1 systemically. As expected, mice treated with anti-PD-1 alone had comparable outcomes to the vehicle-only control group (FIG. 13c). Therapy with p(Man-TLR7-PDS) alone showed significantly higher efficacy over anti-PD-1 monotherapy, but the effect of the combination therapy was even more pronounced. This result is promising for the translational potential of the technology, as it can synergize with checkpoint inhibitors to treat anti-PD-1-resistant tumors.

6. p(Man-TLR7-PDS) Eradicates Established MC38 Colon Carcinoma

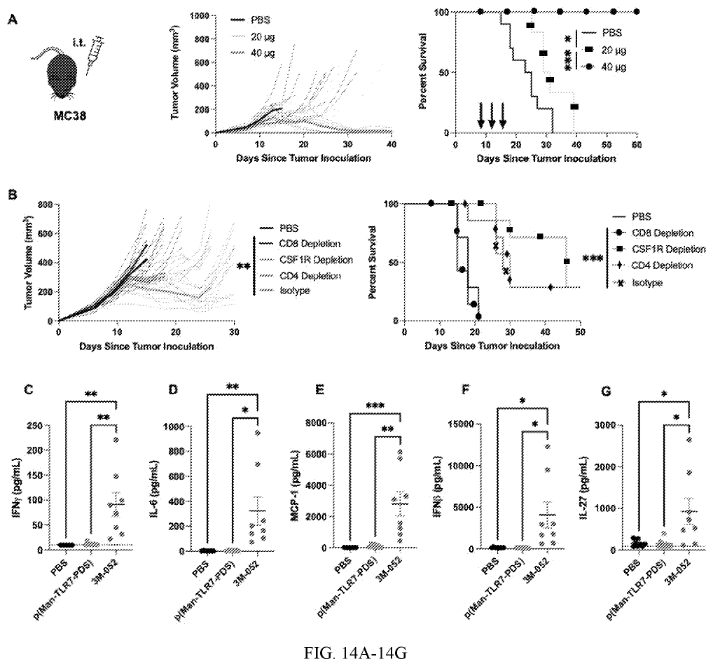

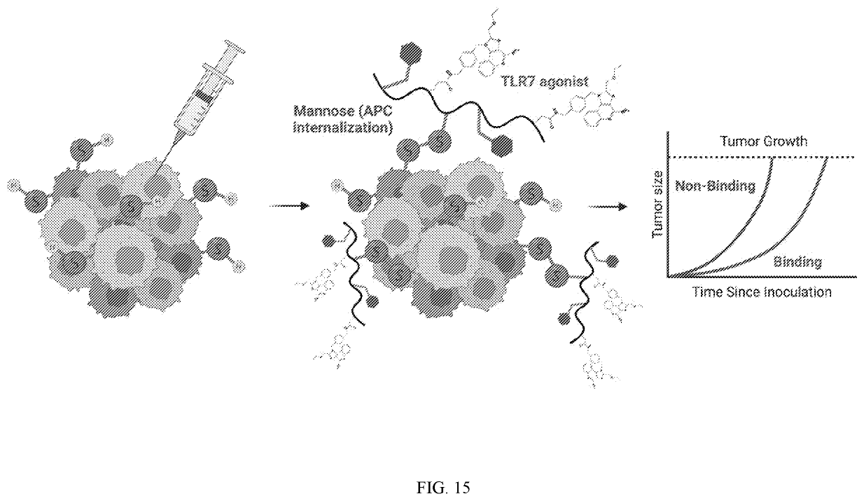

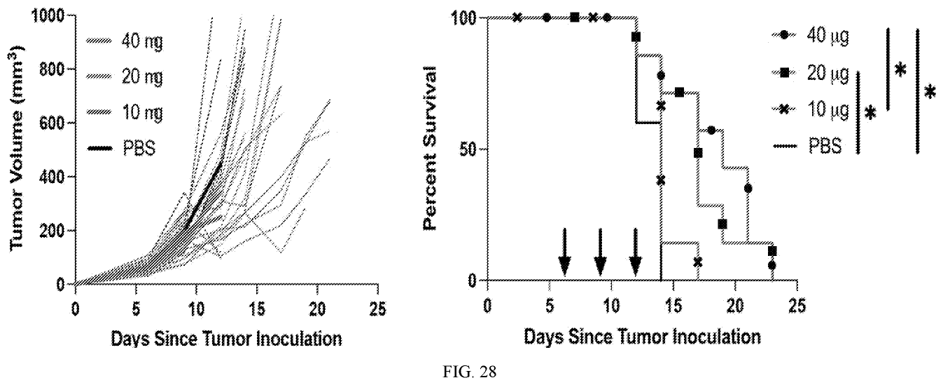

The inventors next moved to evaluate the translational potential of p(Man-TLR7-PDS). To do so, the inventors aimed to establish dose-dependent efficacy of the technology in MC38 colon carcinoma. In this model, p(Man-TLR7-PDS) demonstrated dose-dependent antitumor control and, at the high dose, achieved a 100% survival rate (FIG. 14a). The inventors also confirmed the dose-dependency of p(Man-TLR7-PDS) in B16F10 melanoma (FIG. 28).

7. Antitumor Efficacy of p(Man-TLR7-PDS) is Dependent on CD8⁺ T Cells

To evaluate which immune cell populations were mediating antitumor efficacy, the inventors administered depletion antibodies specific for CD4, CD8α, or colony stimulating factor-1 receptor (CSFIR). In order to observe more significant differences, the inventors waited until tumors reached an average size of ˜100 mm³. The results established a clear dependence on CD8⁺ T cells, as tumor growth for those mice was comparable to that of the PBS-treated group (FIG. 14b), as is observed with many immunotherapies (43). Although previous work demonstrated the importance of CD4⁺-T cell-mediated humoral response in a vaccination setting (22), but in the tumor setting, CD4 depletion did not affect the therapeutic efficacy, likely due to depletion of regulatory T cells. Given that p(Man-TLR7-PDS) can bind both macrophages and dendritic cells (DCs), the dispensable role of macrophages, as indicated by a lack of an ameliorated response to CSFIR depletion, points to DCs in the tumor microenvironment as being the likely target for the adjuvanted tumor cell material (44). It is known that TLR7 agonist-induced antitumor activity can be mediated through DCs (16). However, it is important to note that CSFIR depletion has a complex effect, as it reduces both pro-inflammatory M1-like macrophages as well as anti-inflammatory M2-like tumor associated macrophages (45). Although p(Man-TLR7-PDS) may preferentially bind M2-like macrophages due to their high levels of CD206 expression (46), the interpretation of this result is complex. Nonetheless, these results established that the antitumor efficacy of p(Man-TLR7-PDS) is indispensably mediated by CD8⁺ T cells.

8. Cysteine Binding Limits Systemic Inflammation Associated with TLR7/8 Agonists

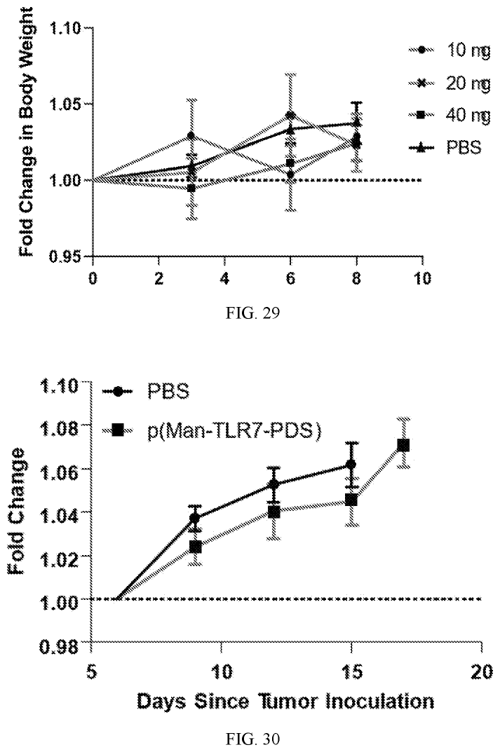

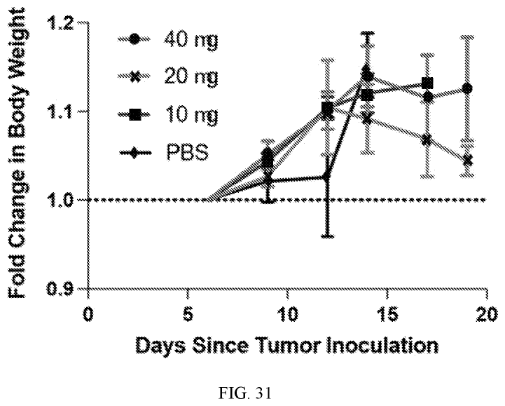

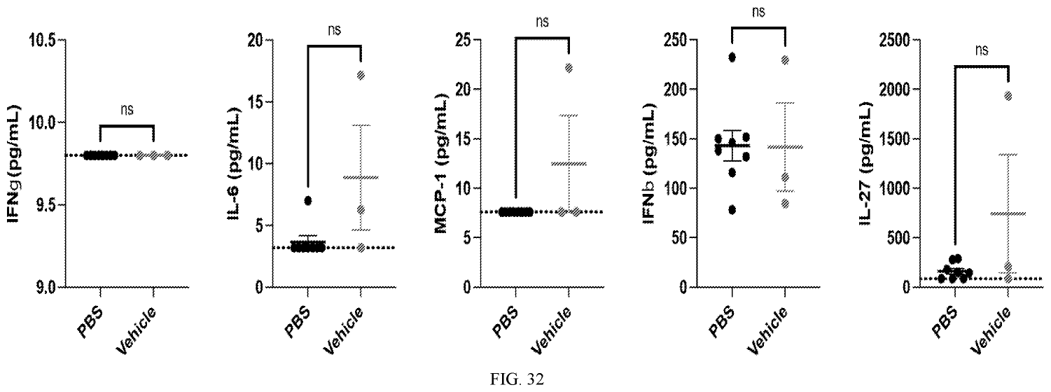

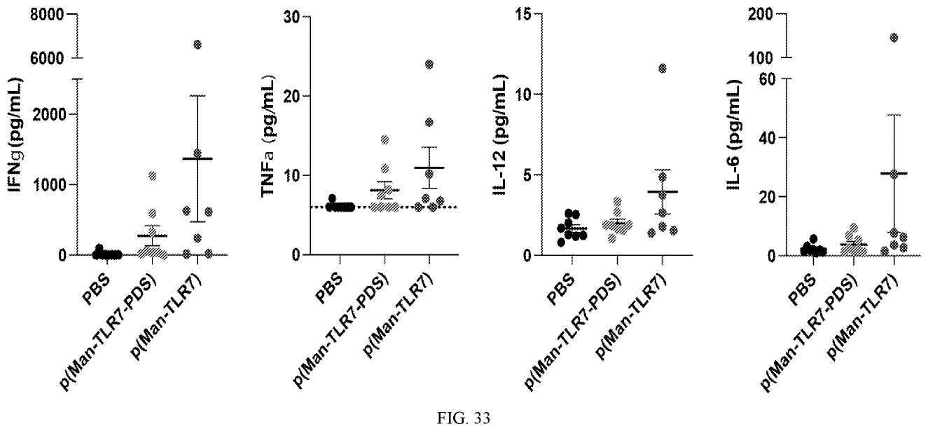

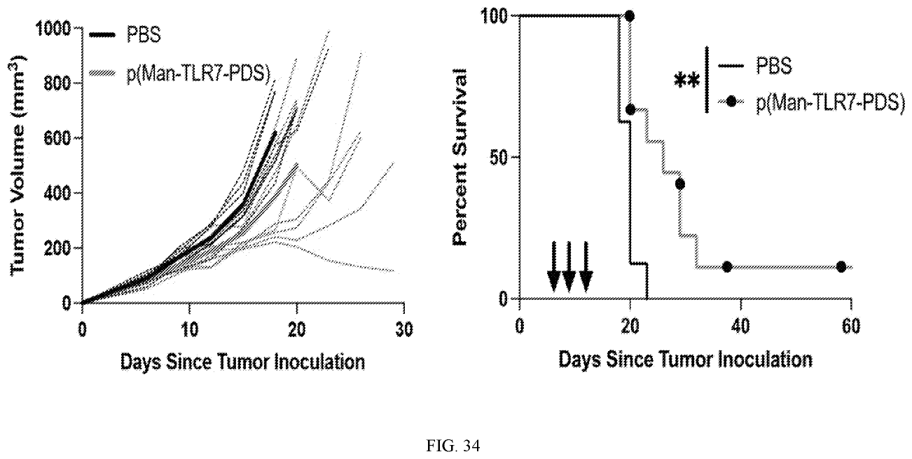

Because thiol binding mediates intratumoral retention and tumor cell binding, it should prevent the documented toxicity associated with the systemic exposure to TLR7 agonists (16, 47). The inventors observed that p(Man-TLR7-PDS) administered subcutaneously in healthy mice and intratumorally in B16F10 melanoma- and CT26 adenocarcinoma-bearing mice did not lead to weight loss (FIG. 29-31). To further characterize the systemic toxicity, the inventors selected 3M-052 (telratolimod), which is a lipid-modified TLR7/8 agonist designed to form depots for controlled release from the injection site (or for incorporation into liposomes), as a benchmark for p(Man-TLR7-PDS) (48). The inventors intratumorally injected MC38 colon carcinoma-bearing mice with either p(Man-TLR7-PDS) or 3M-052 on a TLR7 equimolar basis and evaluated the production of systemic inflammatory cytokines. Here, the inventors show that 3M-052, but not p(Man-TLR7-PDS), produced significant upregulation of interferon gamma (IFN-γ), interleukin 6 (IL-6), monocyte chemoattractant protein 1 (MCP-1), interferon beta (IFN-β), and interleukin 27 (IL-27) as compared to PBS or vehicle control (FIG. 14c-g, FIG. 32). This provides evidence that the systemic toxicity associated with other clinically relevant localized/controlled-release formulations is limited with p(Man-TLR7-PDS) therapy. The inventors validated this result with a similar experiment in B16F10 melanoma, where the inventors compared p(Man-TLR7-PDS) to non-binding p(Man-TLR7) and observed a trend towards reduced cytokine levels (FIG. 33).

### C. Discussion

In this work, the inventors exploit redox imbalance in tumors to create an in situ cancer vaccine, in which a multifunctional polymer is conjugated to the tumor cell surface and tumor debris via exofacial unpaired cysteine, thus adjuvating tumor neoantigens. The polymer is endowed with TLR7 agonizing moieties and with mannose moieties to mark tumor debris for APC endocytosis and to further localize the TLR7 agonist within the endosome, the compartment in which the receptor is active.

By exploiting the fundamental dysregulated metabolic profile of solid tumors, this thiol-binding platform can be applied to virtually all solid tumor types in a tumor-agnostic manner. Importantly, this strategy is clinically relevant as the phenomenon of excessive oxidative stress and redox imbalance is observed in human cancers (32, 51, 52), so the inventors expect that PDS-mediated intratumoral cell binding and retention has high translational potential. The inventors are also encouraged by the modularity of the platform. Other functional monomers can be incorporated, and larger agonists can be conjugated to the azide-terminated chain end (which was otherwise used only for conjugating a fluorophore here). With these approaches, the inventors can use this platform to deliver different adjuvants or create combination therapies with potentially synergistic effects (53).

Other common synthetic anticancer drug delivery strategies can be broadly grouped into two categories. The first are nanocarriers, which traditionally exploit the enhanced permeability and retention (EPR) effect, whereby disordered vasculature allows for nanoscale materials to exit circulation and reduced lymphatic drainage enables their retention. While this effect is strong in small animals, translation to humans has been limited due to accumulation in other organs (54). Current efforts focus on more specific targeting to the tumor (30). The cysteine-reactive platform does not rely on passive accumulation but rather on local retention after intratumoral administration and does not require any specific targeting moieties, such as antitumor antibodies. The other category is intratumoral drug depots, where an injection forms a depot in situ to slowly release a drug over time (55, 56). This platform does not have a temporal-release aspect that requires very specific engineering. It further allows for covalent association of the drug with tumor cell surfaces rather than nonspecific local release, which in this example of an in situ vaccine may be particularly important.

In conclusion, the drug delivery platform described herein is promising for three main reasons. First is its simplicity—the inventors used well-studied thiol-reactive chemistry to exploit a fundamental metabolic feature of solid tumors. The second is its modularity—the immune-active monomers can be substituted with other small molecule agonists, and the chain end can be conjugated to larger adjuvant molecules. Finally, the polymer is translationally relevant—other TLR7/8 agonist formulations are in development and show significant clinical promise. The demonstration the inventors provide here for in situ cancer vaccines shows efficacy in both monotherapy and in combination with checkpoint inhibition and reduction of systemic toxicity of the immune agonist compared to another non-binding depot formulation.

### D. Materials and Methods

### 1. Mice and Cancer Cell Lines

Female C57BL/6 mice (aged 8-12 weeks) were purchased from Charles River Laboratory. Female Balb/c mice (aged 8-12 weeks) were purchased from Jackson Laboratory. B16F10 murine melanoma, CT26 and MC38 murine colon carcinoma, EMT6 murine mammary carcinoma, and MDA-MB-231 human breast adenocarcinoma cell lines were purchased from ATCC and cultured according to instructions, with routine checks for mycoplasma contamination. Tumor inoculations were 500,000 cells in 30 μL sterile PBS unless otherwise noted. Polymer solutions were verified as endotoxin-free prior to injection via HEK-Blue TLR4 reporter cells (InvivoGen). Tumor dimensions were measured with digital calipers and volume was calculated as height×width×thickness×(pi/6). All of the animal experiments performed in this research were approved by the Institutional Animal Care and Use Committee of the University of Chicago under protocol 72456.

### 2. PET-RAFT Polymerization

Briefly, monomers and chain transfer agent (CTA) were dissolved in 1 mL DMSO in a schlenk tube. Eosin Y was added at 0.02 equivalents to CTA in 50 μL DMSO. The tube was sealed and degassed via four freeze-pump thaw cycles then placed inside a foil-wrapped bowl with green LED strip lights. The reaction was covered with foil and left stirring for 14 hours. After that time, the polymer was precipitated in cold diethyl ether three times to remove residual monomer. The resulting polymer was dried under reduced pressure and characterized with ¹H NMR and GPC using InfinityLab EasiVial PMMA standards (Agilent, cat. no. PL2020-0202). Prior to in vivo administration, polymers were dissolved in sterile water and purified using 7 kD MWCO Zeba desalting columns (Thermo Scientific, cat. no. 89883). The process is described in detail with monomer masses used in the Supplemental Methods

3. In Vitro Cancer Cell Binding of p(PDS)

For detection, AZDye 647 DBCO (Click Chemistry Tools, cat. no. 1302-25) was conjugated to polymer via the azide chain end (leading to 1:1 conjugation) and unconjugated dye was removed using Zeba desalting columns with a 7K MWCO (Thermo Scientific, cat. no. 89883). Cells were removed from culture, washed free of media with PBS two times, then incubated on ice with various concentrations of dye labeled polymer for 90 min. For the pre-treatment experiment, cells were treated with 500 μM N-ethyl maleimide (NEM, Sigma Aldrich, cat. no. 04259) or tris(2-carboxyethyl) phosphine (TCEP, Sigma Aldrich, cat. no. 75259) for 30 min and washed twice with PBS prior to polymer incubation. After polymer incubation, cells were washed twice with PBS and resuspended in 2% v/v heat-inactivated fetal bovine serum (FBS, ThermoFisher, cat. no. A3840002) in PBS for flow cytometric analysis. Cells were acquired on BD LSRFortessa and data were analyzed via FlowJo. Mean fluorescence intensity (MFI) of singlet cells was recorded and compared between groups.

### 4. Histological Analysis of Tumor Retention

B16F10 tumor-bearing mice were injected intratumorally with 30 μL dye labeled polymer at a concentration of 200 μM fluorophore once tumors reached an average size of ˜80 mm³. Six hours after injection, tumors were collected and embedded in OCT compound (ThermoFisher, cat. no. 23730571) and frozen at −80° C. before sectioning on a microtome-cryostat into 5 μm sections. The slides were then stained with AlexaFluor 594 conjugated anti-thioredoxin-1 (polyclonal, Novus Biologicals, cat. no. 89458AF594). Slides were then fixed with ProLong Gold antifade reagent with DAPI (Fisher Scientific, cat. no. P36931) and imaged with Olympus IX83 spinning-disc confocal fluorescence microscope (Olympus, Tokyo, Japan).

### 5. Flow Cytometric Analysis of Cell Binding In Vivo

B16F10 tumors were inoculated intradermally into the backs of 8-week-old female C57BL/6 mice as described previously. Once tumors reached an average size of ˜100 mm³, 30 μL AZ647 dye labeled polymer (for FIG. 11: molecular weight matched, containing high or low weight fraction PDS, or none for non-binding control; for FIG. 12: p(Man-TLR7-PDS) or p(Man-TLR7), as used in efficacy studies) was injected intratumorally at a concentration of 200 μM fluorophore. Three hours after injection, tumors were collected and digested for 30 min at 37° C. Digestion medium was pyruvate free DMEM (Gibco, ThermoFisher, cat. no. 10313021) supplemented with 5% FBS, 3.3 mg/mL collagenase D (Sigma Aldrich, cat. no. 11088858001), 1 mg/mL collagenase IV (Worthington Biochemical, Fisher Scientific, cat. no. NC9919937) and 1.2 mM CaCl₂). Single-cell suspensions were prepared using a Corning Falcon70 μm cell strainer (Fisher Scientific, cat. no. 08-771-2). Red blood cells were lysed with 3 mL ACK lysing buffer (Gibco, ThermoFisher, cat. no. A10492-1) for 90 sec and neutralized with 15 mL DMEM supplemented with 5% FBS. Cell viability was determined using LIVE/DEAD Fixable Violet Dead Cell Stain (405 nm excitation, Invitrogen, ThermoFisher, cat. no. L34955). Staining with PE anti-mouse CD45 (clone 30-F11, BioLegend, cat. no. 103106) and Alexa Fluor 488 anti-TRP1 (clone EPR21960, Abcam, cat. no. ab270104) was done in 2% FBS in PBS. Cells were acquired on BD LSRFortessa, and data was analyzed via FlowJo. Polymer MFI and frequency of polymer positive cells of each population and polymer treatment were measured and compared.

6. In Vitro Activity of p(Man-TLR7-PDS)

RAW 264.7 macrophage-like cells were purchased from ATCC and cultured according to instructions. One day after plating in a flat-bottom, non-treated 96 well plate, cells were treated with various concentrations of TLR7 equivalent polymer or R848 (Sigma Aldrich, cat. no. SML0196) (as quantified by absorbance at 327 nm). Supernatant was collected 24 hours after treatment and analyzed via LEGENDplex Mouse Inflammation Panel (BioLegend, cat. no. 740446).

7. Antitumor Efficacy of p(Man-TLR7-PDS) Vs. Non-Binding p(Man-TLR7)

For the melanoma model, B16F10 cells were inoculated intradermally in the shaved left shoulder of 8-week-old female C57BL/6 mice. Mice bearing established tumors were injected intratumorally with 40 μg TLR7 equivalent (as quantified by absorbance at 327 nm, based on the equation: TLR7 content=1.9663*A₃₇₂+0.0517) of either p(Man-TLR7-PDS) or p(Man-TLR7) in 30 μL sterile PBS or vehicle only control on days 6, 9, and 12 after tumor inoculation. The volume of the tumor was recorded as previously described. Mice were euthanized when the tumor volume exceeded 500 mm³ and/or based on humane end-point criteria. For the mammary carcinoma model, EMT6 cells were inoculated into the left mammary fat pad of 8-week-old female BALB/c mice. Mice bearing established tumors were injected intratumorally with 40 μg TLR7 equivalent of either p(Man-TLR7-PDS) or p(Man-TLR7) in 30 μL sterile PBS or vehicle only control on days 6, 9, 12, and 15 after tumor inoculation. The volume of the tumor was recorded as described above. Mice were euthanized when the tumor volume exceeded 750 mm³ and/or based on humane end-point criteria.

8. Antitumor Efficacy of p(Man-TLR7-PDS) in Combination with CPI

8-week-old female Balb/c mice were inoculated with CT26 cells subcutaneously as previously described. On days 6, 9, 12, and 15 after tumor inoculation, mice were injected intratumorally with 40 μg TLR7 equivalent p(Man-TLR7-PDS) in 30 μL sterile PBS or vehicle only control and intraperitoneally with 100 μg anti-PD-1 (29F.1A12, BioXCell, cat. no. BE0273) in 100 μL sterile PBS. Tumor growth was recorded as described above, and mice were euthanized when the tumor volume exceeded 750 mm³ and/or based on humane end-point criteria.

9. Dose Dependent Efficacy of p(Man-TLR7-PDS) in MC38 Colon Carcinoma

8-week-old female C57BL/6 mice were inoculated with MC38 colon carcinoma cells subcutaneously as described above. On days 6, 9, and 12 after tumor inoculation, mice were injected intratumorally with 20 or 40 μg TLR7 equivalent p(Man-TLR7-PDS) in 30 μL sterile PBS or vehicle-only control. Tumor growth was recorded as described above, and mice were euthanized when the tumor volume exceeded 500 mm³ and/or based on humane end-point criteria.

10. Efficacy of p(Man-TLR7-PDS) with Cellular Depletions

8-week-old female C57BL/6 mice were inoculated with MC38 colon carcinoma cells subcutaneously as described above. Depletion antibodies were administered starting one day before treatment, which was started when tumors reached an average size of ˜100 mm³, and were discontinued after treatment. CD4 (clone GK1.5, BioXCell, cat. no. BE0003-1) and CD8α (Clone 2.43 BioXCell, cat. no. BE0061) depletion antibodies and isotype control (BioXCell, cat. no. BE0086) were administered at a dose of 400 μg every three days. CSFIR (clone ASF98, BioXCell, cat. no. BE0213) depletion antibodies were administered at a dose of 300 μg every other day. Treatment with p(Man-TLR7-PDS), 40 μg TLR7 equivalent in 30 μL sterile PBS, occurred every three days for a total of three doses. Tumor growth was recorded as described above, and mice were euthanized when the tumor volume exceeded 500 mm³ and/or based on humane end-point criteria.

### 11. Serum Cytokine Analysis

Mice bearing established MC38 tumors were injected intratumorally with 40 μg TLR7 equivalent (as quantified by absorbance at 273 nm, relative to a standard curve) polymer (full or non-binding control) in 30 μL sterile PBS. As a comparison, other mice were injected with TLR7/8 agonist telratolimod (3M-052, MedChemExpress, cat. no. HY-109104) in 10% DMSO, 40% PEG300 (Sigma Aldrich, cat. no. 8.07484), and 5% Tween-80 in PBS (Sigma Aldrich, cat. no. P8074) (with additional vehicle control mice). Six hours after injection, plasma was collected via submandibular bleed and analyzed for pro-inflammatory cytokines using LEGENDplex Mouse Inflammation Panel Assay (BioLegend, cat. no. 740446).

### 12. Statistical Analysis

Statistical analysis was performed using Prism (v8, GraphPad Prism). For multiple comparisons, one-way analysis of variance followed by Tukey post hoc test was used. For direct comparisons, unpaired t-test was used. For survival, pairwise log rank (Mantel-Cox) tests were used.

### 13. Monomer and Polymer Synthesis

All chemicals were reagent grade and purchased from Sigma Aldrich and used as received unless otherwise noted. 2-(2-(2-(2-azidoethoxy) ethoxy) ethoxy)ethyl 4-cyano-4 (((dodecylthio)carbonthioyl)thio)pentanoate (4), N-[2-(α-D-mannose)ethyl]methacrylamide (7), and N-(6-((4-((4-amino-2-(ethoxymethyl)-1H-imidazo[4,5-c]quinolin-1yl)methyl)benzyl)amino) hexyl) methacrylamide (8) were synthesized as previously described by Wilson et al. (2019). All other protocols listed in the following pages. All NMR spectra were collected on a Bruker Avance III HD Nanobay 400 MHz NMR unless otherwise noted and NMR spectra were analyzed with MNova (MestreLab). Gel permeation chromatography was performed using Tosoh EcoSEC size exclusion chromatography system with Tosoh SuperAW3000+Tosoh SuperAW4000 columns, eluted in DMF+0.01 M LiBr at 50° C. Mass spectrometry analysis was performed on Agilent 6130 LCMS using positive mode direct flow inject analysis.

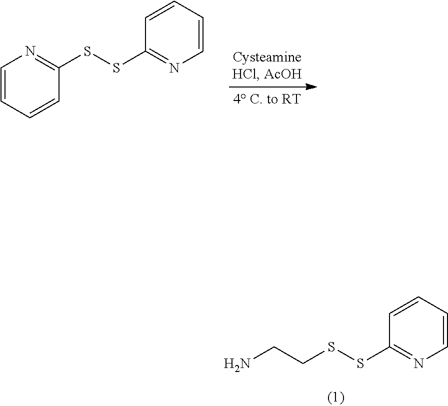 [SMILES: c1ccc(SSc2ccccn2)nc1 ; NCCSSc1ccccn1]

2-(pyridin-2-yldisulfaneyl) ethan-1-amine (1). 2,2′-Dipyridyldisulfide (4.4 g, 20 mmol) was dissolved in 20 mL methanol with 0.4 mL acetic acid (6.98 mmol) on ice with constant stirring. To that solution, cysteamine hydrochloride (1.2 g, 15.5 mmol) in 10 mL methanol was added dropwise over 15 min. After 24 hours, crude product was precipitated 4 times in cold diethyl ether then dried overnight under vacuum, providing 3.62 g (97.3%) of pure white powder. C₇H₁₀N₂S₂, ESI-MS [M+H]⁺ₜₕₑₒᵣ=m/z 187.037, [M+H]⁺_{found}=187.1 m/z. ¹H NMR (400 MHz, CD₃OD) δ 8.55 (d, 1H), 7.81 (m, 1H), 7.86 (d, 1H), 7.33 (m, 1H), 3.15 (t, 2H), 3.05 (t, 2H).

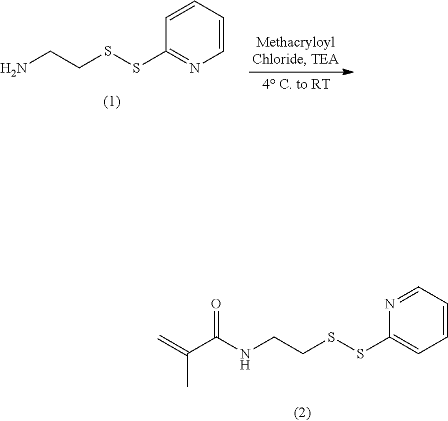 [SMILES: NCCSSc1ccccn1 ; C=C(C)C(=O)NCCSSc1ccccn1]

N-(2-(pyridine-2-yldisulfaneyl)ethyl) methacrylamide (PDS monomer) (2). Compound 1 (2 g, 10.8 mmol) was dissolved in 10 mL DCM with 3 mL triethylamine (21.6 mmol) and stirred on ice. Methacryloyl chloride (1.35 g, 13.0 mmol) in 2 mL DCM was added dropwise. The reaction was left to react overnight, then extracted with water and DCM three times. The organic phase was dried over magnesium sulfate, filtered, and adsorbed on silica. The crude product was purified via flash column chromatography (Hex:EtOAc 1:2 v/v) to give 1.6 g (61.1%) of pure product: C₁₁H₁₄N₂OS₂, ESI-MS [M+H]⁺ₜₕₑₒᵣ=m/z 255.063, [M+H]⁺_{found}=255.1 m/z. 1H NMR (400 MHz,D₂O) δ 8.42 (d, 1H), 7.83 (q, 2H), 7.25 (d, 1H), 5.74 (s, 1H), 5.42 (s, 1H), 3.52 (q, 2H), 3.01 (t, 2H), 1.96 (s, 3H).

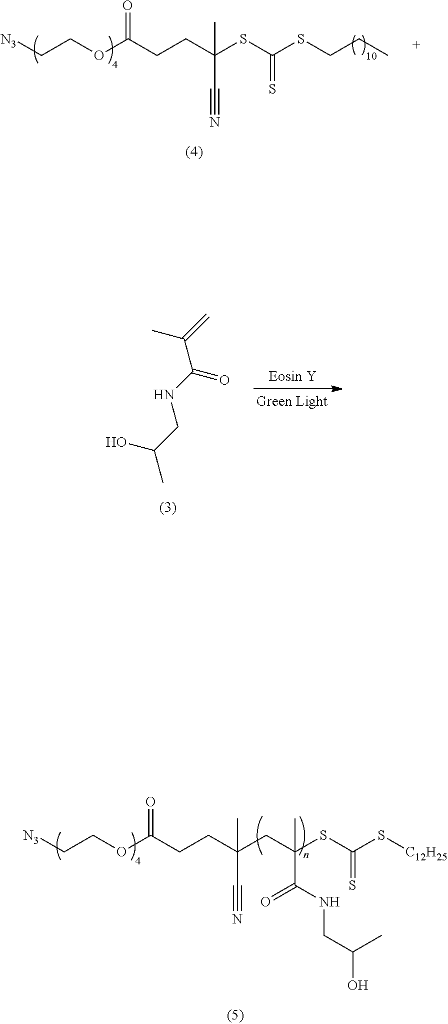

p(HPMA) (5). N-(2-Hydroxypropyl) methacrylamide (HPMA) (3) was recrystallized in acetone and dried prior to use. HPMA (3) (215 mg, 1.51 mmol) was dissolved in 1 mL DMSO with azide-functionalized trithiocarbonate chain transfer agent (CTA) (4) (13 mg, 0.0215 mmol) in a schlenk tube. Photo initiator Eosin Y (0.298 mg, 0.00043 mmol) was added in 50 μL DMSO. The tube was sealed and degassed via four freeze-pump thaw cycles then placed inside a foil wrapped bowl with green LED strip lights. The reaction was covered with foil and left stirring for 14 hours. After that time, the polymer was precipitated in cold diethyl ether three times to remove residual monomer. The resulting polymer (150 mg) was dried under reduced pressure and characterized with 1H NMR and GPC using PMMA standards. The p(HPMA) used in binding studies had a number averaged molecular weight of 7,339 and a dispersity of 1.327. Resulting polymer was fluorescently conjugated and used for in vitro and in vivo characterization of cell binding and uptake.

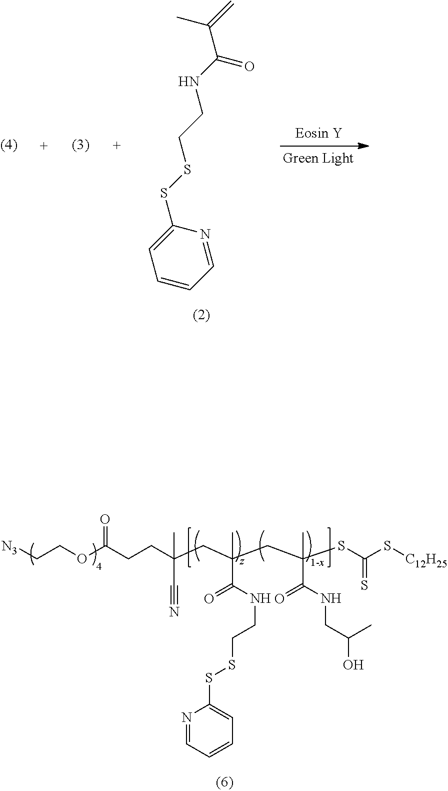

p(PDS) (6). HPMA (3) (200 mg, 1.40 mmol), PDS monomer (2) (68.6 mg, 0.27 mmol), and CTA (4) (16.3 mg, 0.027 mmol) were dissolved in 1 mL DMSO in a schlenk tube. Eosin Y (0.374 mg, 0.00054 mmol) was added in 50 μL DMSO. The tube was sealed and degassed via four freeze-pump thaw cycles then placed inside a foil-wrapped bowl with green LED strip lights. The reaction was covered with foil and left stirring for 14 hours. After that time, the polymer was precipitated in cold diethyl ether three times to remove residual monomer. The resulting polymer (150 mg) was dried under reduced pressure and characterized with ¹H NMR and GPC using PMMA standards. The p(PDS) used in binding studies had a number averaged molecular weight of 8,197 and a dispersity of 1.387. Using ¹H NMR, it was determined that the polymer had an average of 8.0 PDS monomers per chain. The low PDS polymer used in the in vivo binding experiment was synthesized with half of the PDS monomer, with HPMA replaced in a weight equivalent. This polymer had a number averaged molecular weight of 7.972 and a dispersity of 1.255, with 3.1 PDS monomers per chain. Resulting polymer was fluorescently conjugated and used for in vitro and in vivo characterization of cell binding and uptake.

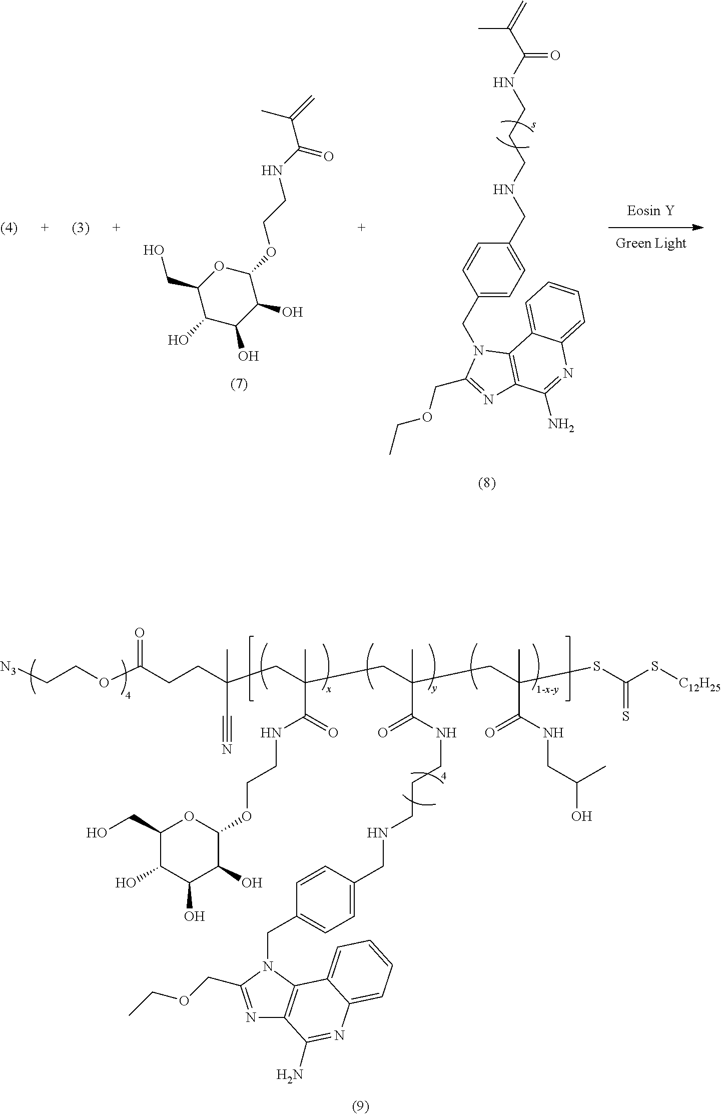

p(Man-TLR7) (9). HPMA (3) (68.4 mg, 0.478 mmol), mannose monomer (7) (43.8 mg, 0.151 mmol), TLR7 monomer (8) (40 mg, 0.70 mmol), and CTA (4) (4.2 mg, 0.0069 mmol) were dissolved in 1 mL DMSO in a schlenk tube. Eosin Y (0.09 mg, 0.00013 mmol) was added in 50 μL DMSO. The tube was sealed and degassed via four freeze-pump thaw cycles then placed inside a foil-wrapped bowl with green LED strip lights. The reaction was covered with foil and left stirring for 14 hours. After that time, the polymer was precipitated in cold diethyl ether three times to remove residual monomer. The resulting polymer (70 mg) was dried under reduced pressure and characterized with ¹H NMR and GPC using PMMA standards. Resulting polymer was used as a non-binding control in in vitro and in vivo activity and retention experiments.

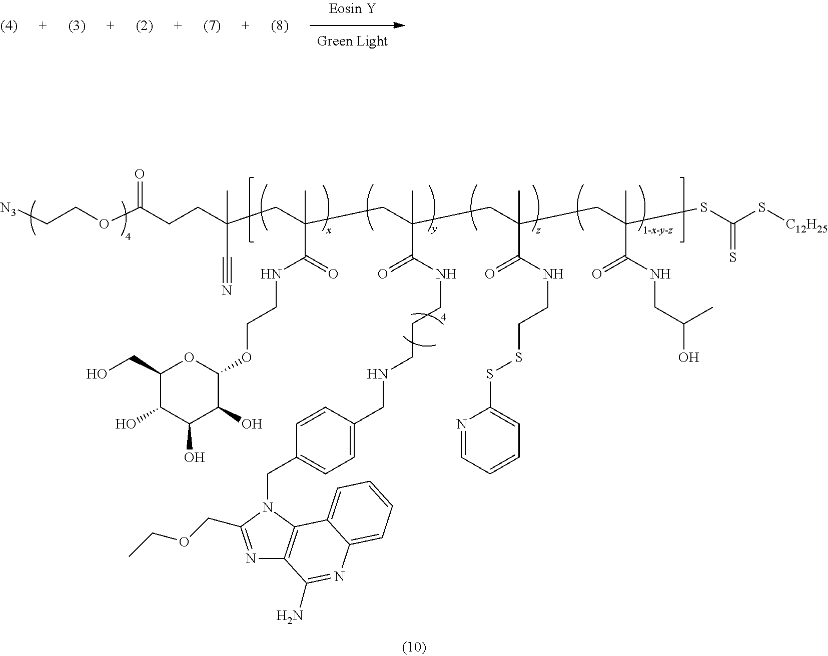

p(Man-TLR7-PDS) (10). HPMA (3) (56.5 mg, 0.395 mmol), PDS monomer (2) (19.9 mg, 0.079 mmol), mannose monomer (7) (49.3 mg, 0.169 mmol), TLR7 monomer (8) (45 mg, 0.078 mmol), and CTA (4) (4.8 mg, 0.0079 mmol) were dissolved in 1 mL DMSO in a schlenk tube. Eosin Y (0.102 mg, 0.00015 mmol) was added in 50 μL DMSO. The tube was sealed and degassed via four freeze-pump thaw cycles then placed inside a foil-wrapped bowl with green LED strip lights. The reaction was covered with foil and left stirring for 14 hours. After that time, the polymer was precipitated in cold diethyl ether three times to remove residual monomer.

The resulting polymer (100 mg) was dried under reduced pressure and characterized with ¹H NMR and GPC using PMMA standards. Resulting polymer was used in in vitro and in vivo activity and retention experiments.

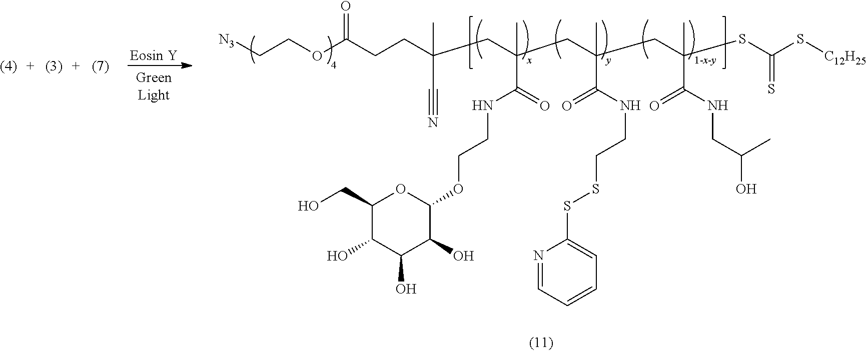

p(Man-PDS) (11). HPMA (3) (100 mg, 0.699 mmol), PDS monomer (2) (35.3 mg, 0.139 mmol), mannose monomer (7) (87.2 mg, 0.299 mmol) and CTA (4) (8.4 mg, 0.014) mmol) were dissolved in 1 mL DMSO in a schlenk tube. Eosin Y (0.18 mg, 0.00028 mmol) was added in 50 μL DMSO. The tube was sealed and degassed via four freeze-pump thaw cycles then placed inside a foil-wrapped bowl with green LED strip lights. The reaction was covered with foil and left stirring for 14 hours. After that time, the polymer was precipitated in cold diethyl ether three times to remove residual monomer. The resulting polymer (100 mg) was dried under reduced pressure and characterized with ¹H NMR and GPC using PMMA standards. Resulting polymer was used as a non-adjuvanted control in in vitro and in vivo activity experiments and was fluorescently conjugated and used for in vitro characterization of cell binding and uptake.

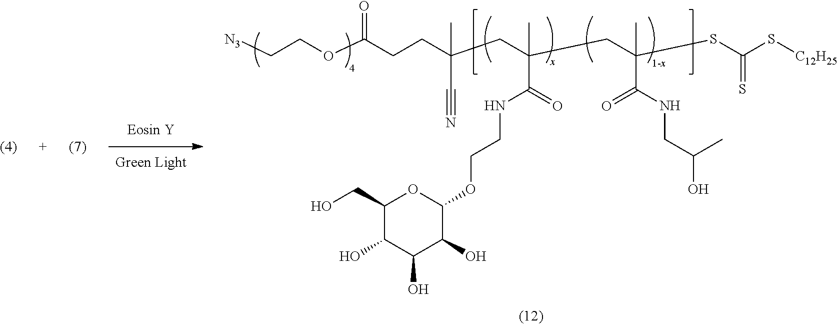

p(Man) (12). HPMA (3) (104 mg, 0.727 mmol), mannose monomer (7) (59.1 mg, 0.202 mmol) and CTA (4) (7.6 mg, 0.013) mmol) were dissolved in 1 mL DMSO in a schlenk tube. Eosin Y (0.16 mg, 0.00025 mmol) was added in 50 μL DMSO. The tube was sealed and degassed via four freeze-pump thaw cycles then placed inside a foil-wrapped bowl with green LED strip lights. The reaction was covered with foil and left stirring for 14 hours. After that time, the polymer was precipitated in cold diethyl ether three times to remove residual monomer. The resulting polymer (100 mg) was dried under reduced pressure and characterized with 1H NMR and GPC using PMMA standards. Resulting polymer was fluorescently conjugated and used for in vitro characterization of cell binding and uptake.

### E. References

The following references and the publications referred to throughout the specification, to the extent that they provide exemplary procedural or other details supplementary to those set forth herein, are specifically incorporated herein by reference.
(1) Hanahan, D. Hallmarks of Cancer: New Dimensions. Cancer Discov. 2022, 12 (1), 31-46.
(2) Gatenby, R. A.; Gillies, R. J. Why do cancers have high aerobic glycolysis? Nat. Rev. Cancer 2004, 4 (11), 891-899.
(3) Li, F.; Simon, M. C. Cancer cells don't live alone: metabolic communication within tumor microenvironments. Dev. Cell 2020, 54 (2), 183-195.
(4) Fukumura, D.; Jain, R. K. Tumor microenvironment abnormalities: causes, consequences, and strategies to normalize. J. Cell. Biochem. 2007, 101 (4), 937-949.
(5) Khan, M. A.; Zubair, H.; Anand, S.; Srivastava, S. K.; Singh, S.; Singh, A. P. Dysregulation of metabolic enzymes in tumor and stromal cells: Role in oncogenesis and therapeutic opportunities. Cancer Lett. 2020, 473, 176-185.
(6) Mansurov, A.; Hosseinchi, P.; Chang, K.; Lauterbach, A. L.; Gray, L. T.; Alpar, A. T.; Budina, E.; Slezak, A. J.; Kang. S.; Cao, S.; et al. Masking the immunotoxicity of interleukin-12 by fusing it with a domain of its receptor via a tumour-protease-cleavable linker. Nat. Biomed. Eng. 2022, 1-11.
(7) Haslam, A.; Prasad, V. Estimation of the percentage of US patients with cancer who are eligible for and respond to checkpoint inhibitor immunotherapy drugs. JAMA Netw. Open 2019, 2 (5), e192535-e192535.
(8) Carlino, M. S.; Larkin, J.; Long. G. V. Immune checkpoint inhibitors in melanoma. Lancet 2021, 398 (10304), 1002-1014.
(9) Kwa, M. J.; Adams, S. Checkpoint inhibitors in triple-negative breast cancer (TNBC): Where to go from here. Cancer 2018, 124 (10), 2086-2103.
(10) Rodell, C. B.; Arlauckas, S. P.; Cuccarese, M. F.; Garris, C. S.; Li, R.; Ahmed, M. S.; Kohler, R. H.; Pittet, M. J.; Weissleder, R. TLR7/8-agonist-loaded nanoparticles promote the polarization of tumour-associated macrophages to enhance cancer immunotherapy. Nat. Biomed. Eng. 2018, 2 (8), 578-588.
(11) Schön, M.; Schön, M. TLR7 and TLR8 as targets in cancer therapy. Oncogene 2008, 27 (2), 190-199.
(12) Dowling. D. J. Recent advances in the discovery and delivery of TLR7/8 agonists as vaccine adjuvants. Immunohorizons 2018, 2 (6), 185-197.
(13) Prins, R. M.; Craft, N.; Bruhn, K. W.; Khan-Farooqi, H.; Koya, R. C.; Stripecke, R.; Miller, J. F.; Liau, L. M. The TLR-7 agonist, imiquimod, enhances dendritic cell survival and promotes tumor antigen-specific T cell priming: relation to central nervous system antitumor immunity. J. Immunol. 2006, 176 (1), 157-164.
(14) Dockrell, D. H.; Kinghorn, G. R. Imiquimod and resiquimod as novel immunomodulators. J. Antimicrob. Chemother. 2001, 48 (6), 751-755.
(15) Frega, G.; Wu, Q.; Le Naour, J.; Vacchelli, E.; Galluzzi, L.; Kroemer, G.; Kepp, O. Trial Watch: experimental TLR7/TLR8 agonists for oncological indications. Oncoimmunology 2020, 9 (1), 1796002.
(16) Smits, E. L.; Ponsaerts, P.; Berneman, Z. N.; Van Tendeloo, V. F. The use of TLR7 and TLR8 ligands for the enhancement of cancer immunotherapy. Oncologist 2008, 13 (8), 859-875.
(17) Varshney, D.; Qiu, S. Y.; Graf, T. P.; McHugh, K. J. Employing drug delivery strategies to overcome challenges using TLR7/8 agonists for cancer immunotherapy. AAPS J. 2021, 23 (4), 1-18.
(18) Banjac, A.; Perisic, T.; Sato, H.; Seiler, A.; Bannai, S.; Weiss, N.; KöIle, P.; Tschoep, K.; Issels. R.; Daniel, P. The cystine/cysteine cycle: a redox cycle regulating susceptibility versus resistance to cell death. Oncogene 2008, 27 (11), 1618-1628.
(19) Ceccarelli, J.; Delfino, L.; Zappia, E.; Castellani, P.; Borghi, M.; Ferrini, S.; Tosetti, F.; Rubartelli, A. The redox state of the lung cancer microenvironment depends on the levels of thioredoxin expressed by tumor cells and affects tumor progression and response to prooxidants. Int. J. Cancer 2008, 123 (8), 1770-1778.
(20) Chaiswing. L.; Zhong, W.; Cullen, J. J.; Oberley, L. W.; Oberley, T. D. Extracellular redox state regulates features associated with prostate cancer cell invasion. Cancer Res. 2008, 68 (14), 5820-5826.
(21) Jonas, C. R.; Ziegler, T. R.; Gu, L. H.; Jones, D. P. Extracellular thiol/disulfide redox state affects proliferation rate in a human colon carcinoma (Caco2) cell line. Free Radic. Biol. Med. 2002, 33 (11), 1499-1506.
(22) Wilson, D. S.; Hirosue, S.; Raczy, M. M.; Bonilla-Ramirez, L.; Jeanbart, L.; Wang, R.; Kwissa, M.; Franetich, J.-F.; Broggi, M. A.; Diaceri, G. Antigens reversibly conjugated to a polymeric glyco-adjuvant induce protective humoral and cellular immunity. Nat. Mater. 2019, 18 (2), 175-185.
(23) Altinbasak. I.; Arslan, M.; Sanyal, R.; Sanyal, A. Pyridyl disulfide-based thiol-disulfide exchange reaction: shaping the design of redox-responsive polymeric materials. Polym. Chem. 2020.
(24) El-Sayed, M. E.; Hoffman, A. S.; Stayton, P. S. Rational design of composition and activity correlations for pH-sensitive and glutathione-reactive polymer therapeutics. J. Control. Release 2005, 101 (1-3), 47-58.
(25) Bulmus, V.; Woodward, M.; Lin. L.; Murthy, N.; Stayton, P.; Hoffman, A. A new pH-responsive and glutathione-reactive, endosomal membrane-disruptive polymeric carrier for intracellular delivery of biomolecular drugs. J. Control. Release 2003, 93 (2), 105-120.
(26) Ju. Y.; Xing. C.; Wu, D.; Wu, Y.; Wang, L.; Zhao, H. Covalently Connected Polymer-Protein Nanostructures Fabricated by a Reactive Self-Assembly Approach. Chem. Eur. J. 2017, 23 (14), 3366-3374.
(27) Allegrezza, M. L.; Konkolewicz, D. PET-RAFT Polymerization: Mechanistic Perspectives for Future Materials. ACS Macro Lett. 2021, 10 (4), 433-446.
(28) Xu. J.; Shanmugam, S.; Duong, H. T.; Boyer, C. Organo-photocatalysts for photoinduced electron transfer-reversible addition-fragmentation chain transfer (PET-RAFT) polymerization. Polym. Chem. 2015, 6 (31), 5615-5624.
(29) Ji, X.; Liu, J.; Liu, L.; Zhao, H. Enzyme-polymer hybrid nanogels fabricated by thiol-disulfide exchange reaction. Colloids Surf. B. Biointerfaces 2016, 148, 41-48.
(30) Nakamura, H.; Nakamura, K.; Yodoi, J. Redox regulation of cellular activation. Annu. Rev. Immunol. 1997, 15 (1), 351-369.
(31) Holmgren, A.; Lu, J. Thioredoxin and thioredoxin reductase: current research with special reference to human disease. Biochem. Biophys. Res. Commun. 2010, 396 (1), 120-124.
(32) Powis, G.; Kirkpatrick, D. L. Thioredoxin signaling as a target for cancer therapy. Curr. Opin. Pharmacol. 2007, 7 (4), 392-397.
(33) Gray, L. T.; Raczy, M. M.; Briquez, P. S.; Marchell, T. M.; Alpar, A. T.; Wallace, R. P.; Volpatti, L. R.; Sasso, M. S.; Cao, S.; Nguyen, M.; et al. Generation of potent cellular and humoral immunity against SARS-COV-2 antigens via conjugation to a polymeric glyco-adjuvant. Biomaterials 2021, 278, 121159.
(34) Ma, F.; Zhang. J.; Zhang. J.; Zhang, C. The TLR7 agonists imiquimod and gardiquimod improve DC-based immunotherapy for melanoma in mice. Cell. Mol. Immunol. 2010, 7 (5), 381-388.
(35) Michaelis, K. A.; Norgard, M. A.; Zhu, X.; Levasseur, P. R.; Sivagnanam, S.; Liudahl, S. M.; Burfeind, K. G.; Olson, B.; Pelz, K. R.; Ramos, D. M. A. The TLR7/8 agonist R848 remodels tumor and host responses to promote survival in pancreatic cancer. Nat. Comm. 2019, 10 (1), 1-15.
(36) Fan, W.; Yang. X.; Huang, F.; Tong, X.; Zhu, L. Identification of CD206 as a potential biomarker of cancer stem-like cells and therapeutic agent in liver cancer. Oncol. Lett. 2019, 18 (3), 3218-3226.
(37) Yan, H.; Kamiya, T.; Suabjakyong, P.; Tsuji, N. M. Targeting C-type lectin receptors for cancer immunity. Front. Immunol. 2015, 6, 408.
(38) Jaynes, J. M.; Sable, R.; Ronzetti, M.; Bautista, W.; Knotts, Z.; Abisoye-Ogunniyan, A.; Li, D.; Calvo, R.; Dashnyam, M.; Singh, A. Mannose receptor (CD206) activation in tumor-associated macrophages enhances adaptive and innate antitumor immune responses. Sci. Transl. Med. 2020, 12 (530), eaax6337.
(39) Zarif, J. C.; Baena-Del Valle, J. A.; Hicks, J. L.; Heaphy, C. M.; Vidal, I.; Luo, J.; Lotan, T. L.; Hooper, J. E.; Isaacs, W. B.; Pienta, K. J. Mannose Receptor-positive Macrophage Infiltration Correlates with Prostate Cancer Onset and Metastatic Castration-resistant Disease. Eur. Urol. Oncol. 2019, 2 (4), 429-436.
(40) Vinod, N.; Hwang, D.; Azam, S. H.; Van Swearingen, A. E.; Wayne, E.; Fussell, S. C.; Sokolsky-Papkov, M.; Pecot. C. V.; Kabanov, A. V. High-capacity poly(2-oxazoline) formulation of TLR 7/8 agonist extends survival in a chemo-insensitive, metastatic model of lung adenocarcinoma. Sci. Adv. 2020, 6 (25), caba5542.
(41) Chen, L.; Han, X. Anti-PD-1/PD-L1 therapy of human cancer: past, present, and future. J. Clin. Invest. 2015, 125 (9), 3384-3391.
(42) Yu, J. W.; Bhattacharya, S.; Yanamandra, N.; Kilian, D.; Shi, H.; Yadavilli, S.; Katlinskaya, Y.; Kaczynski, H.; Conner, M.; Benson, W. Tumor-immune profiling of murine syngeneic tumor models as a framework to guide mechanistic studies and predict therapy response in distinct tumor microenvironments. PLOS One 2018, 13 (11), e0206223.
(43) Raskov, H.; Orhan, A.; Christensen, J. P.; Gögenur, I. Cytotoxic CD8+ T cells in cancer and cancer immunotherapy. Br. J. Cancer 2021, 124 (2), 359-367.
(44) Noy, R.; Pollard, J. W. Tumor-associated macrophages: from mechanisms to therapy. Immunity 2014, 41 (1), 49-61.
(45) O'Brien, S. A.; Orf, J.; Skrzypczynska, K. M.; Tan, H.; Kim, J.; DeVoss, J.; Belmontes, B.; Egen, J. G. Activity of tumor-associated macrophage depletion by CSFIR blockade is highly dependent on the tumor model and timing of treatment. Cancer Immunol., Immunother. 2021, 70 (8), 2401-2410.
(46) Xu. Z.-J.; Gu, Y.; Wang. C.-Z.; Jin, Y.; Wen, X.-M.; Ma, J.-C.; Tang. L.-J.; Mao, Z.-W.; Qian. J.; Lin, J. The M2 macrophage marker CD206: a novel prognostic indicator for acute myeloid leukemia. Oncoimmunology 2020, 9 (1), 1683347.
(47) Chi, H.; Li, C.; Zhao, F. S.; Zhang, L.; Ng. T. B.; Jin, G.; Sha, O. Anti-tumor activity of toll-like receptor 7 agonists. Front. Pharmacol. 2017, 8, 304.
(48) Smirnov, D.; Schmidt, J. J.; Capecchi, J. T.; Wightman, P. D. Vaccine adjuvant activity of 3M-052: an imidazoquinoline designed for local activity without systemic cytokine induction. Vaccine 2011, 29 (33), 5434-5442.
(49) Digilio, G.; Catanzaro, V.; Fedeli, F.; Gianolio, E.; Menchise, V.; Napolitano, R.; Gringeri, C.; Aime, S. Targeting exofacial protein thiols with Gd III complexes. An efficient procedure for MRI cell labelling. Chem. Commun. 2009, (8), 893-895.
(50) Menchise, V.; Digilio, G.; Gianolio, E.; Cittadino, E.; Catanzaro, V.; Carrera, C.; Aime, S. In vivo labeling of B16 melanoma tumor xenograft with a thiol-reactive gadolinium based MRI contrast agent. Mol. Pharm. 2011, 8 (5), 1750-1756.
(51) Chaiswing. L.; Bourdeau-Heller, J. M.; Zhong, W.; Oberley. T. D. Characterization of redox state of two human prostate carcinoma cell lines with different degrees of aggressiveness. Free Radic. Biol. Med. 2007, 43 (2), 202-215.
(52) Cook, J. A.; Gius, D.; Wink, D. A.; Krishna, M. C.; Russo, A.; Mitchell, J. B. Oxidative stress, redox, and the tumor microenvironment. In Seminars in Radiation Oncology, 2004; Elsevier: Vol. 14, pp 259-266.
(53) Gondan, A. I. B.; Ruiz-de-Angulo, A.; Zabaleta, A.; Blanco, N. G.; Cobaleda-Siles, B. M.; García-Granda, M. J.; Padro, D.; Llop, J.; Arnaiz, B.; Gato, M. Effective cancer immunotherapy in mice by polyIC-imiquimod complexes and engineered magnetic nanoparticles. Biomaterials 2018, 170, 95-115.
(54) Nehoff, H.; Parayath, N. N.; Domanovitch, L.; Taurin, S.; Greish, K. Nanomedicine for drug targeting: strategies beyond the enhanced permeability and retention effect. Int. J. Nanomed. 2014, 9, 2539.
(55) Liu, W.; Mackay, J. A.; Dreher, M. R.; Chen, M.; McDaniel, J. R.; Simnick, A. J.; Callahan, D. J.; Zalutsky, M. R.; Chilkoti, A. Injectable intratumoral depot of thermally responsive polypeptide-radionuclide conjugates delays tumor progression in a mouse model. J. Control. Release 2010, 144 (1), 2-9.
(56) Fakhari, A.; Subramony, J. A. Engineered in-situ depot-forming hydrogels for intratumoral drug delivery. J. Control. Release 2015, 220, 465-475.
(57) Vacchelli, E.; Galluzzi, L.; Eggermont, A.; Fridman, W. H.; Galon, J.; Sautès-Fridman, C.; Tartour, E.; Zitvogel, L.; Kroemer, G. Trial watch: FDA-approved Toll-like receptor agonists for cancer therapy. Oncoimmunology 2012, 1 (6), 894-907.
(58) Lynn, G. M.; Sedlik, C.; Baharom, F.; Zhu, Y.; Ramirez-Valdez, R. A.; Coble, V. L.; Tobin, K.; Nichols, S. R.; Itzkowitz, Y.; Zaidi, N. Peptide-TLR-7/8a conjugate vaccines chemically programmed for nanoparticle self-assembly enhance CD8 T-cell immunity to tumor antigens. Nat. Biotechnol. 2020, 38 (3), 320-332.
(59) Barrat, F. J. TLR8: No gain, no pain. J. Exp. Med. 2018, 215 (12), 2964-2966.
(60) Cervantes, J. L.; Weinerman, B.; Basole, C.; Salazar, J. C. TLR8: the forgotten relative revindicated. Cell. Mol. Immunol. 2012, 9 (6), 434-438.
(61) Ackerman, S. E.; Pearson, C. I.; Gregorio, J. D.; Gonzalez, J. C.; Kenkel, J. A.; Hartmann, F. J.; Luo, A.; Ho, P. Y.; LeBlanc, H.; Blum, L. K. Immune-stimulating antibody conjugates elicit robust myeloid activation and durable antitumor immunity. Nat. Cancer 2021, 2 (1), 18-33.
(62) Neve. R.; Lane, H.; Hynes, N. The role of overexpressed HER2 in transformation. Ann. Oncol. 2001, 12, S9-S13.
(63) Bojarczuk, K.; Siernicka, M.; Dwojak, M.; Bobrowicz, M.; Pyrzynska, B.; Gaj, P.; Karp, M.; Giannopoulos, K.; Efremov, D.; Fauriat, C. B-cell receptor pathway inhibitors affect CD20 levels and impair antitumor activity of anti-CD20 monoclonal antibodies. Leukemia 2014, 28 (5), 1163-1167.
(64) Davda, J.; Declerck, P.; Hu-Lieskovan, S.; Hickling. T. P.; Jacobs, I. A.; Chou, J.; Salek-Ardakani, S.; Kraynov, E. Immunogenicity of immunomodulatory, antibody-based, oncology therapeutics. J. Immunother. Cancer 2019, 7 (1), 1-9.
(65) Champiat, S.; Tselikas, L.; Farhane, S.; Raoult, T.; Texier, M.; Lanoy, E.; Massard, C.; Robert, C.; Ammari, S.; De Bacre, T. Intratumoral immunotherapy: from trial design to clinical practice. Clin. Cancer. Res. 2021, 27 (3), 665-679.
(66) Ngwa, W.; Irabor, O. C.; Schoenfeld, J. D.; Hesser, J.; Demaria, S.; Formenti, S. C. Using immunotherapy to boost the abscopal effect. Nat. Rev. Cancer 2018, 18 (5), 313-322.
(67) Suck, N.; Campesato, L. F.; Merghoub, T.; Khalil, D. N. Targeted APC activation in cancer immunotherapy to enhance the abscopal effect. Front. Immunol. 2019, 10, 604.

### Example 4—Cancer Vaccines with Direct Adjuvant Binding

Based on promising results from the intratumoral setting, the inventors chose to investigate the efficacy of tumor binding adjuvants, specifically p(Man-TLR7-PDS), for the treatment of blood cancers like acute myeloid leukemia (AML). Here, intravenous injection mimics intratumoral injection, where the polymer binds circulating tumor cells and debris. This serves to adjuvant the cells and target the antigens therein to APCs in the spleen and liver, where a protective antigen-specific immune response can be initiated. Our data details the ability of PDS-containing polymers to bind tumor cells and induce an effective immune response in combination with standard of care chemotherapy cytarabine in a murine model of AML.

### A. Methods and Results

### 1. Mice and Cancer Cell Lines

Female C57BL/6 mice or B6.SJL-Ptprca Pepcb/BoyJ (CD45.1) were purchased from Charles River Laboratory at the age of 8-10 weeks. C1498 AML cells were purchased from ATCC and cultured according to instructions. Tumor inoculations were 1,000,000 cells in 100 μL sterile PBS injected intravenously. Polymer (synthesized as previously described) injections were 40 μg TLR7 equivalent p(Man-TLR7-PDS) in 100 μL PBS injected intravenously, and cytarabine was 2 mg (roughly 100 mpk) in 100 μL PBS injected intraperitoneally. Mice were monitored for signs of disease progression and sacrificed at humane end points based on symptomology. Cell lines were routinely checked for mycoplasma contamination.

2. In Vitro Cancer Cell Binding of p(PDS-HPMA)

The inventors first evaluated whether the PDS-containing polymer could successfully bind exofacial protein thiols on tumor cells in vitro using comparable methods as for solid tumor cells. They also investigated the effect of cytarabine pre-treatment on polymer binding, both in a dose and time dependent manner.

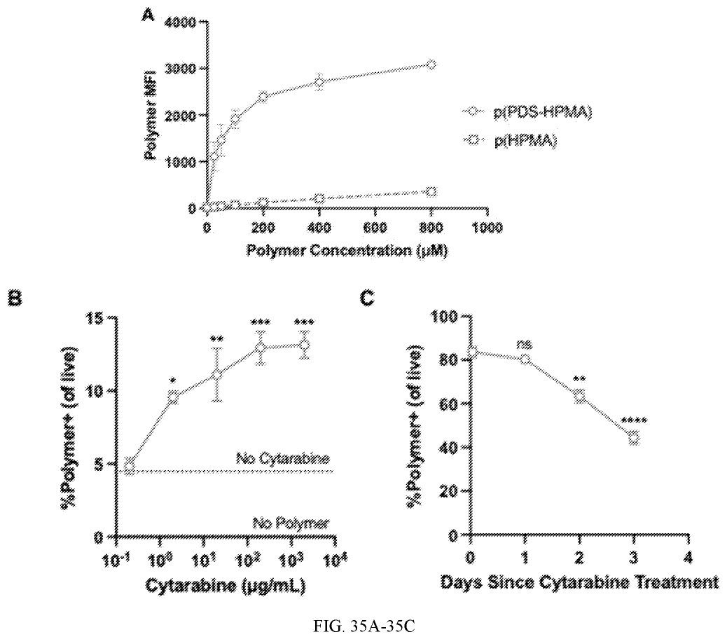

Importantly, binding was observed to be polymer concentration-dependent and significantly higher for the PDS containing polymer, p(PDS-HPMA), than for the non-binding control p(HPMA) (FIG. 35A). The inventors also observed that cytarabine pre-treatment for 4 hours prior to treatment increased the efficiency of polymer binding in a dose-dependent manner (FIG. 35B). Finally, they determined that the cytarabine, if given too far from polymer treatment, decreases binding efficiency of PDS-containing polymers (FIG. 35C). Together, these results support the combination of cytarabine with cancer-binding polymers, particularly when given in close sequential order.

3. Ex Vivo Cancer Cell Binding of p(PDS-HPMA)

The inventors next evaluated ex vivo binding to cancer cells via flow cytometry. Two days after C1498 inoculation into CD45.1 mice, blood was collected via submandibular bleed. White blood cells were isolated by first removing the serum after centrifugation then performing three rounds of ACK lysis (150 μL for 5 minutes). Isolated WBCs were washed with PBS and incubated with fluorescently-labeled p(PDS-HPMA) on ice for 90 minutes. After incubation, cells were washed twice with PBS and resuspended in 2% heat inactivated FBS in PBS for flow cytometric analysis. Cell viability was determined using LIVE/DEAD Fixable Violet Dead Cell Stain, and cell populations were determined using PE anti-mouse CD45.1 and BUV395 anti-mouse CD45. C1498 cells were identified as CD45+CD45.1− and endogenous immune cells were double positive.

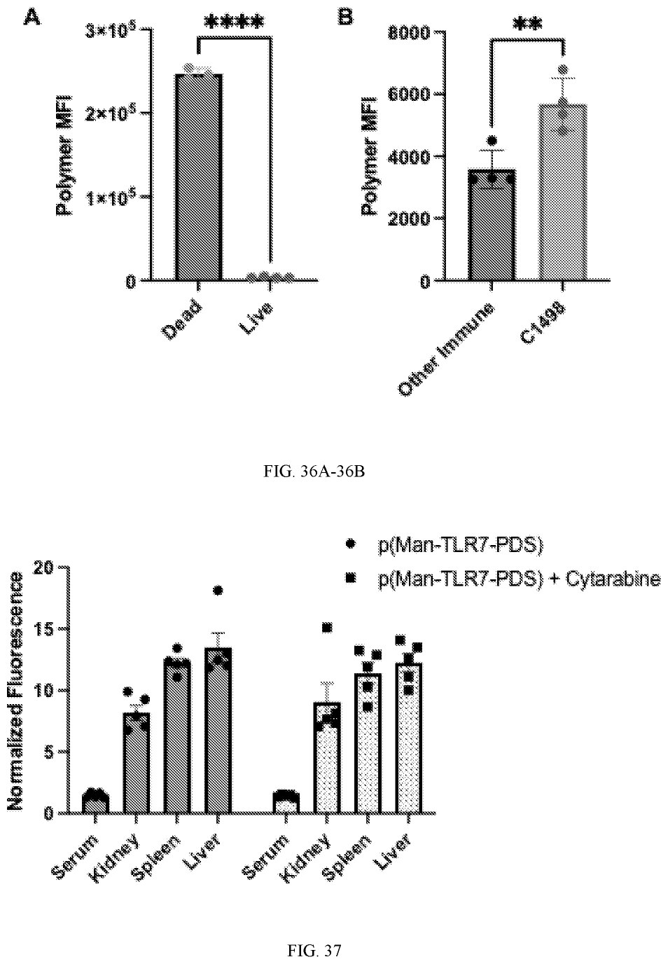

The inventors were primarily interested in confirming preferential binding of PDS-containing polymers to cancer cells in the blood. In this ex vivo assay, it was determined that there is significantly greater binding to those cells over healthy blood immune cells (FIG. 36B). The inventors also showed that the polymer binds dead cells much more efficiently than live cells (FIG. 36A), promoting the hypothesis of adjuvating not only live tumor cells but also cellular debris, which can be induced via direct killing by cytarabine.

### 4. In Vivo Polymer Biodistribution

The inventors next wanted to understand which tissues the full therapeutic polymer was retained in upon intratumoral injection. To achieve this, they inoculated normal B6 mice with C1498. After one day, one group of mice received a dose of cytarabine. After two days all mice were injected dye-labeled p(Man-TLR7-PDS) intravenously. Eight hours after this injection, mice were sacrificed, and tissues were harvested for homogenization and subsequent fluorescence quantification using a microplate reader. Fluorescence values were normalized per mg of tissue.

Based on the data, the inventors can conclude that the polymer primarily accumulates in the liver (FIG. 37). While fluorescence levels between the spleen and liver were comparable, it is important to note that the liver homogenate represent only a small subsection of the liver (<10%) and that the spleen homogenate contained the entire spleen. The inventors also see that cytarabine pre-treatment does not significantly alter the tissue biodistribution of polymer.

5. Anticancer Efficacy of p(Man-TLR7-PDS)

The inventors next moved to evaluate the anticancer efficacy of p(Man-TLR7-PDS) in the therapeutic model of AML, in which C1498 bearing mice are treated and monitored until they reach sacrifice criteria. In the first iteration of this study, mice were inoculated on day 0. On day 1, some mice received cytarabine i.p. On day 2, some received p(Man-TLR7-PDS) i.v. This dosing schedule repeated weekly for four weeks total. Body weights were recorded to monitor tolerability of the therapy.

Subsequently, the inventors wanted to further characterize the efficacy and the requests for the observed effect. In one experiment, they evaluated the importance of both the cytarabine combination and of the TLR7 component of the polymer. In a similar study, they dosed only a single injection of cytarabine and p(Man-TLR7-PDS).

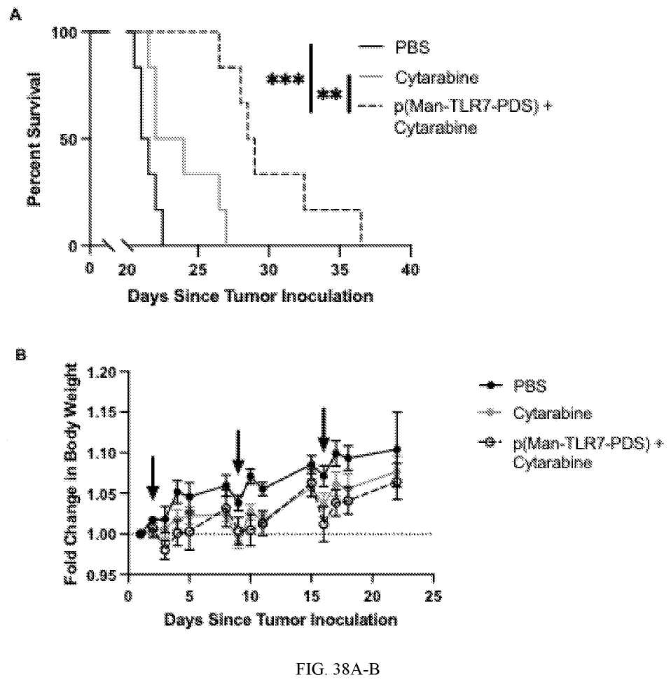

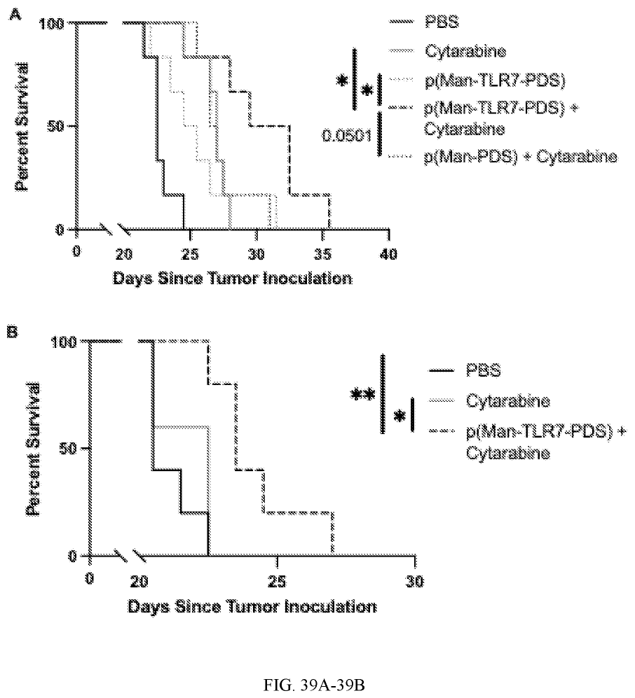

Taken together, these data demonstrate the safety (FIG. 38A) and tolerability (FIG. 38B) of p(Man-TLR7-PDS) combination therapy with cytarabine. The inventors confirmed the efficacy of immune-active components of therapy (FIG. 39A) and further established reduced but still significant efficacy in a one-dose setting (FIG. 39B).

6. Cellular Phenotypes in p(Man-TLR7-PDS)

Finally, the inventors aimed to understand important cellular players mediating anticancer efficacy in our therapy. They analyzed B cell responses using an on-cell ELISA to detect anti-C1498 cell antibodies. Briefly, surviving C1498-bearing mice were bled on day 21, prior to the fourth polymer treatment. C1498 cells were incubated with 10% plasma in PBS for 60 minutes on ice, washed twice with PBS, and then incubated with AF-647-labeled rat anti-mouse-IgG for 30 minutes on ice. After washing, the cells were collected via flow cytometry. In a separate experiment, the inventors analyzed T cell populations in the spleen on day 19, four days after the third polymer injection. Upon sacrifice, single-cell suspensions were obtained by gently disrupting the tissues through a 70 mm cell strainer. Red blood cells were lysed with ACK lysing buffer. Cells were stained with LIVE/DEAD Fixable Violet Dead Cell Stain, BUV395 anti-mouse CD3, FITC anti-mouse-PD-1, PE anti-mouse CD45, APC anti-mouse CD137, and APC-Cy7 anti mouse CD8 and collected via flow cytometry.

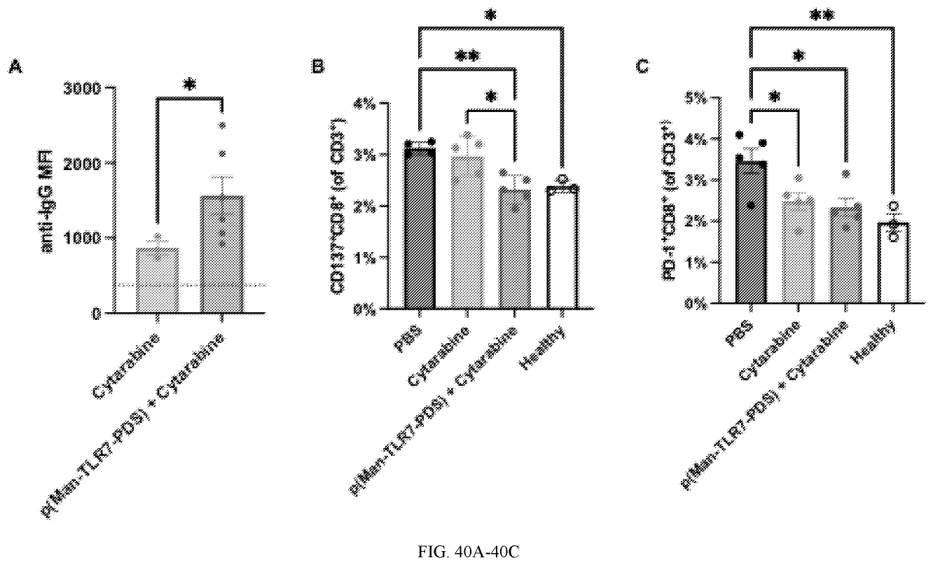

Together, these data suggest the involvement of both T and B cell responses in the prolongation of survival of AML mice. The presence of anticancer antibodies enable antibody dependent cytotoxicity (ADCC) (FIG. 40A). Further, the specific reduction of CD137 and PD-1 expressing CD8 T cells back to healthy levels suggests healthier CD8 T cell function (FIG. 40B,C).

Although certain embodiments have been described above with a certain degree of particularity, or with reference to one or more individual embodiments, those skilled in the art could make numerous alterations to the disclosed embodiments without departing from the scope of this invention. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments. Any reference to a patent publication or other publication is a herein a specific incorporation by reference of the disclosure of that publication. The claims are not to be interpreted as including means-plus- or step-plus-function limitations, unless such a limitation is explicitly recited in a given claim using the phrase(s) “means for” or “step for.” respectively.

1. A polymer comprising the structure (I): 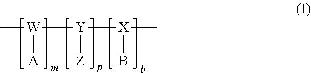
W, Y, and X are each independently a monomer unit of a polymer;
A comprises a group that binds to an Antigen Presenting Cell (APC) mannose receptor;
Z comprises a TLR or STING agonist;
B comprises a pyridyl disulfide moiety or a maleimide moiety;
m is an integer ranging from 0 to 150;
p is an integer ranging from 0 to 10; and
b is an integer ranging from 1 to 10.

2. The polymer of claim 1, wherein W, Y, and X are each independently polymerized monomer units of polyacrylate, a polyacrylamide, a saturated polyolefin, a polyamide, a peptide, a polypeptide, an unsaturated olefin formed by ring opening metathesis polymerization, a siloxane, a polysiloxane, a polyether, a polysaccharide, a polyoxazoline, a polyimine, a polyvinyl derivative or any combination thereof.

3. The polymer of claim 1 or 2, where W, Y, and X are independently selected from acrylic acid, methacrylic acid, acrylamide, methacrylamide, N-(2-hydroxypropyl) methacrylamide, N-(2-hydroxyethyl) methacrylamide, natural and un-natural amino acids, silanols, monosaccharides, glycols, substituted and unsubstituted 2-oxazolines N-substituted and unsubstituted aziridine, substituted and unsubstituted 2-ethyl-2-oxazoline.

4. The polymer of any one of claims 1-3, wherein the TLR agonist has the general structure (II), 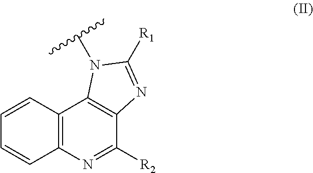
wherein R₁ and R₂ are each independently a hydrogen atom, a halogen, an alkyl group, a substituted alkyl group, a heteroalkyl group, a substituted heteroalkyl group, a cycloalkyl group, a substituted cycloalkyl group, a heterocycloalkyl group, a substituted heterocycloalkyl group, an aryl group, a substituted aryl group, a heteroaryl group, a substituted heteroaryl group, an alkoxy group, an alkoxyalkyl group, an alkoxyalkoxy group, and alkoxyalkoxyalkyl group, an amino group, or a hydroxyl group.

5. The polymer of any one of claims 1-4, wherein moiety 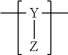
is further defined as 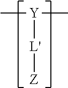
wherein L′ is a substituted or unsubstituted alkyl group comprising from 1 to 9 carbon atoms, a substituted or unsubstituted alkyl group comprising from 1 to 9 carbon atoms, or an ethylene glycol moiety comprising from 1 to 9 ethylene glycol groups.

6. The polymer of any one of claims 1-5, wherein Z is selected from:

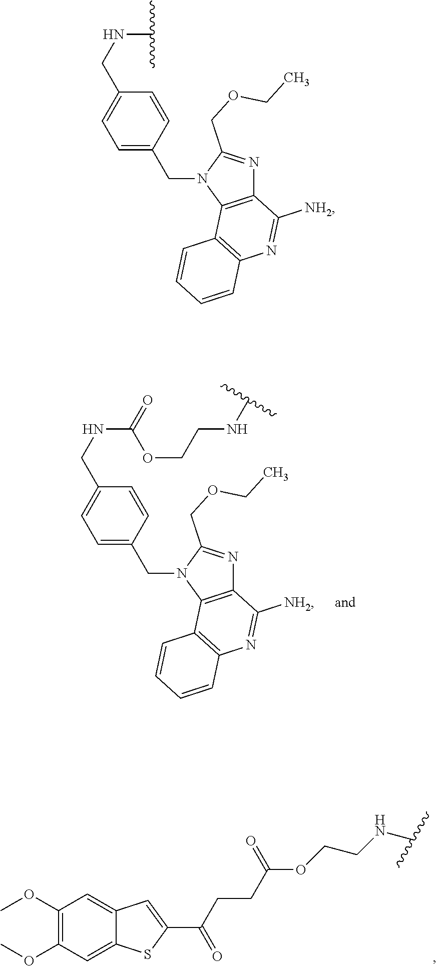 [SMILES: *NCc1ccc(Cn2c(COCC)nc3c(N)nc4ccccc4c32)cc1 ; *NCCOC(=O)NCc1ccc(Cn2c(COCC)nc3c(N)nc4ccccc4c32)cc1 ; *NCCOC(=O)CCC(=O)c1cc2cc(OC)c(OC)cc2s1]

7. The polymer of any one of claims 1-6, wherein

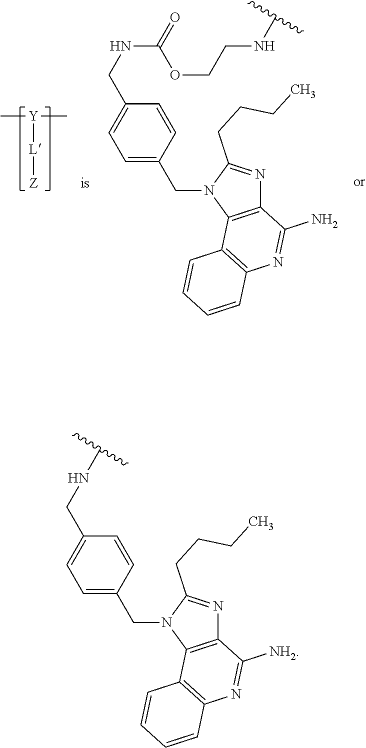

8. The polymer of any one of claims 1-7, wherein

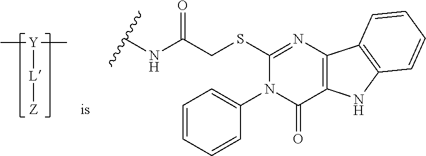

9. The polymer of any one of claims 1-7, wherein 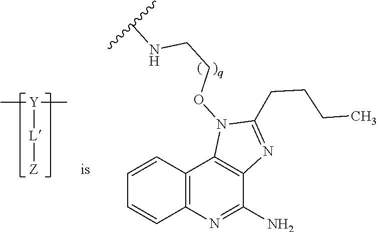
wherein q is 2 to 9.

10. The polymer of any one of claims 1-9, wherein moiety 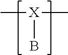
is further defined as

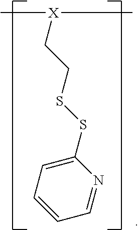

11. The polymer of any one of claims 1-9, wherein moiety 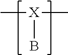
is further defined as

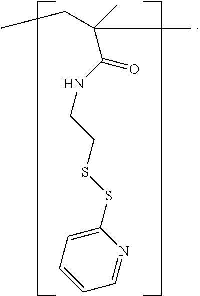

12. The polymer of any one of claims 1-11, wherein moiety 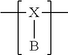
is further defined as

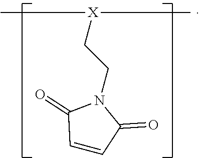

13. The polymer of any one of claims 1-12, wherein moiety 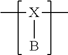
is further defined as

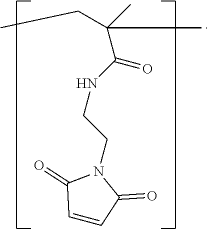

14. The polymer of any one of claims 1-13, wherein moiety 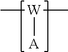
is further defined as

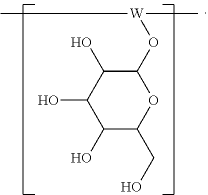

15. The polymer of any one of claims 1-14, wherein moiety 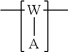
is further defined as

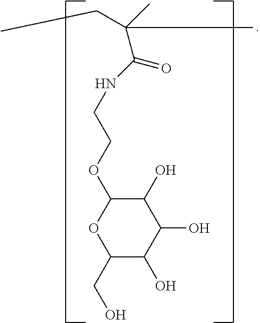

16. The polymer of any one of claims 1-15, wherein moiety 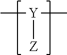
is further defined as 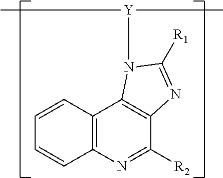
wherein R₁ and R₂ are each independently a hydrogen atom, a halogen, an alkyl group, a substituted alkyl group, a heteroalkyl group, a substituted heteroalkyl group, a cycloalkyl group, a substituted cycloalkyl group, a heterocycloalkyl group, a substituted heterocycloalkyl group, an aryl group, a substituted aryl group, a heteroaryl group, a substituted heteroaryl group, an alkoxy group, an alkoxyalkyl group, an alkoxyalkoxy group, and alkoxyalkoxyalkyl group, an amino group, or a hydroxyl group.

17. The polymer of any one of claims 1-16, wherein moiety 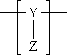
is further defined as

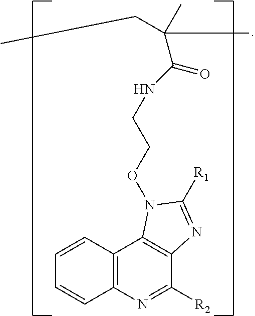

18. The polymer of any one of claims 1-17, wherein the polymer comprises at least one TLR or STING agonist and at least one group that binds to an Antigen Presenting Cell (APC) mannose receptor.

19. The polymer of any one of claims 1-18, wherein the polymer is further defined as 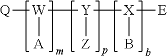
wherein E and Q are end units and each independently comprises a residue of the polymer, a fluorescent molecule, an albumin polypeptide, a dithioate group, an azide, a linker, an immunomodulating agent, a pyridyl disulfide moiety, a maleimide moiety, a TLR agonist, a STING agonist, a group that binds to an Antigen Presenting Cell (APC) mannose receptor, or combinations thereof.

20. The polymer of claim 19, wherein the dithioate group is further defined as 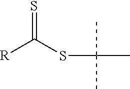
wherein R is an alkyl or aryl group containing from 1 to 12 carbon atoms.

21. The polymer of claim 19, wherein the linker comprises a polyethylene glycol moiety or a hydrocarbon moiety.

22. The polymer of claim 21, wherein the polyethylene glycol linker comprises from 1 to 150 polyethylene glycol units.

23. The polymer of claim 21, wherein the hydrocarbon moiety comprises from 1 to 150 methylene units.

24. The polymer of any one of claims 1-23, wherein the polymer is a copolymer, wherein W, X, and/or Y are different.

25. The polymer of any one of claims 1-24, wherein W, X, and/or Y are the same.

26. The polymer of any one of claims 1-25, wherein m is at least 1, p=0, and at least one of Q or E comprises a TLR or STING agonist.

27. The polymer of any one of claims 1-26, wherein the TLR agonist comprises a CpG TLR agonist.

28. The polymer of claim 27, wherein the CpG TLR agonist comprises ODN1826.

29. The polymer of claim 27 or 28, wherein the CpG TLR agonist comprises a phoshorothioated backbone.

30. The polymer of any one of claims 1-29, wherein W, Y, and X can be provided in any order.

31. A compound comprising: 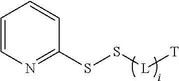
wherein L is a linker, T is a TLR or STING agonist, and i is 0 or 1.

32. The compound of claim 31, wherein the linker comprises a polyethylene glycol moiety or a hydrocarbon moiety.

33. The compound of claim 32, wherein the polyethylene glycol linker comprises from 1 to 150 polyethylene glycol units.

34. The compound of claim 32, wherein the hydrocarbon moiety comprises from 1 to 150 methylene units.

35. The compound of claim 32, wherein the compound is further defined as 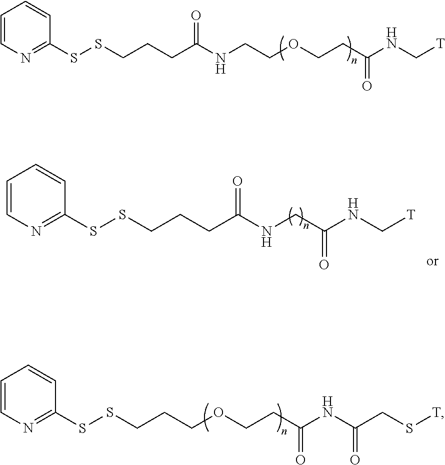
wherein n is from 1 to 150.

36. A compound comprising: 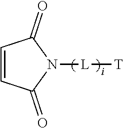
wherein L is a linker, T is a TLR or STING agonist, and i is 0 or 1.

37. The compound of claim 36, wherein the linker comprises a polyethylene glycol moiety or a hydrocarbon moiety.

38. The compound of claim 37, wherein the polyethylene glycol linker comprises from 1 to 150 polyethylene glycol units.

39. The compound of claim 37, wherein the hydrocarbon moiety comprises from 1 to 150 methylene units.

40. The compound of claim 37, wherein the compound is further defined as 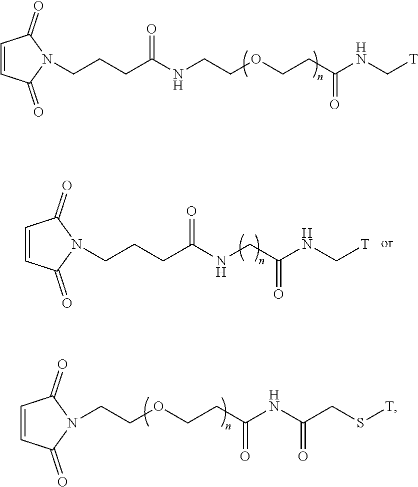
wherein n is from 1 to 150.

41. The compound of any one of claims 31-38, wherein the TLR agonist has the general structure (II), 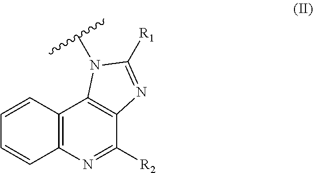
wherein R₁ and R₂ are each independently a hydrogen atom, a halogen, an alkyl group, a substituted alkyl group, a heteroalkyl group, a substituted heteroalkyl group, a cycloalkyl group, a substituted cycloalkyl group, a heterocycloalkyl group, a substituted heterocycloalkyl group, an aryl group, a substituted aryl group, a heteroaryl group, a substituted heteroaryl group, an alkoxy group, an alkoxyalkyl group, an alkoxyalkoxy group, and alkoxyalkoxyalkyl group, an amino group, or a hydroxyl group.

42. The compound of any one of claims 31-40, wherein T is

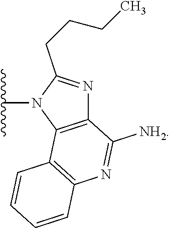 [SMILES: *n1c(CCCC)nc2c(N)nc3ccccc3c21]

43. The compound of any one of claims 31-40, wherein T is

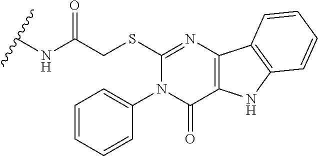 [SMILES: *NC(=O)CSc1nc2c([nH]c3ccccc32)c(=O)n1-c1ccccc1]

44. The compound of any one of claims 31-40, wherein T is

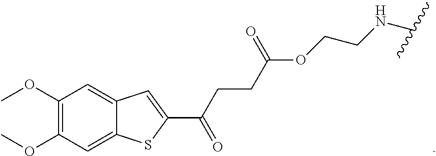 [SMILES: *NCCOC(=O)CCC(=O)c1cc2cc(OC)c(OC)cc2s1]

45. The compound of any one of claims 31-39, wherein the TLR agonist comprises a CpG TLR agonist.

46. The compound of claim 45, wherein the CpG TLR agonist comprises ODN1826.

47. The compound of claim 46 or 47, wherein the CpG TLR agonist comprises a phoshorothioated backbone.

48. The polymer or compound of any one of claims 1-47, wherein the polymer or compound excludes conjugation to a tumor-targeting peptide or further tumor targeting moiety.

49. A composition comprising the polymer or compound of any one of claims 1-48.

50. A method for treating cancer in a subject comprising administering the polymer or compound of any one of claims 1-48 or the composition of claim 49 to the subject.

51. A method for targeting a TLR or STING agonist to a tumor in a subject comprising administering the polymer or compound of any one of claims 1-48 or the composition of claim 49 to the subject.

52. The method of claim 51, wherein the subject has cancer.

53. The method of any one of claims 50-52, wherein the cancer comprises melanoma, a B-cell malignancy, bladder, breast, mammary carcinoma, or colon cancer.

54. The method of claim 53, wherein the B-cell malignancy comprises lymphoma or leukemia.

55. The method of claim 54, wherein the leukemia comprises acute myeloid leukemia.

56. The method of any one of claims 50-55, wherein the cancer comprises a solid tumor.

57. A method for increasing the accumulation of a TLR or STING agonist in a tumor in a subject, the method comprising administering the polymer or compound of any one of claims 1-48 or the composition of claim 49 to the subject.

58. The method of any one of claims 50-57, wherein the method further comprises administration of one or more additional cancer therapies.

59. The method of any one of claims 50-58, wherein the subject has or will receive an immunotherapy.

60. The method of any one of claims 50-59, wherein the method further comprises administration of an immunotherapy.

61. The method of claim 60, wherein the immunotherapy is administered before, after, or concurrent with the compound.

62. The method of any one of claims 59-61, wherein the immunotherapy comprises checkpoint inhibitor therapy.

63. The method of claim 62, wherein the checkpoint inhibitor therapy comprises a PD-1 antibody, a CTLA4 antibody, or both.

64. The method of any one of claims 59-61, wherein the immunotherapy comprises a CD40 agonist.

65. The method of any one of claims 58-64, wherein the additional therapy comprises one or more of cytarabine, daunorubicin, idarubicin, cladribine, fludarabine, mitoxantrone, etoposide, 6-thioguanine, hydroxyurea, prednisone, dexamethasone, methotrexate, 6-mercaptopurine, azacytidine, and decitabine.

66. The method of claim 65, wherein the additional therapy comprises cytarabine.

67. The method of any one of claims 58-66, wherein the additional therapy is administered before the compound or compositions.

68. The method of any one of claims 58-66, wherein the additional therapy is administered after the compound or composition.

69. The method of any one of claims 58-68, wherein the additional therapy is administered within 12 hours of the compound or composition.

70. The method of any one of claims 50-69, wherein the additional therapy, immunotherapy, compound, and/or composition are administered at a dose of at least once per week for a period of time.

71. The method of claim 70 wherein the period of time is 1 month to 12 months.

72. The method of any one of claims 50-71, wherein the polymer, compound, or composition is administered systemically.

73. The method of claim 72, wherein the polymer, compound, or composition is administered by intravenous injection.

74. The method of any one of claims 50-71, wherein the polymer, compound, or composition is administered intratumorally or peritumorally.

75. The method of any one of claims 50-74, wherein the subject is a human subject.


SEQUENCE LISTING (6 sequences)

SEQ ID NO: 1 - protein, 609 aa, synthetic construct [sequence omitted]

SEQ ID NO: 2 - protein, 584 aa, synthetic construct
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 3 - protein, 608 aa, synthetic construct
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 4 - protein, 584 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALG

SEQ ID NO: 5 - protein, 4 aa, synthetic construct
GGGS

SEQ ID NO: 6 - protein, 8 aa, synthetic construct
GGGSGGGS